(19) United States
(12) Patent Application Publication
(10) Pub. No.: US 2025/0346852 A1
(43) Pub. Date: Nov. 13, 2025

(54) Engineered Photosynthetic Organisms

(71) Applicant: Phycobloom Limited, London (GB)

(72) Inventors: Ian HU, London (GB); John Christopher WAITE, London (GB)

(73) Assignee: Phycobloom Limited, London (GB)

(21) Appl. No.: 18/854,342

(22) PCT Filed: Apr. 6, 2023
(86) PCT No.: PCT/GB2023/050930

§ 371 Date: Oct. 4, 2024

(30) Foreign Application Priority Data
Apr. 7, 2022 (GB) 2205111.4

(51) Int. Cl. C12N 1/12; C07K 14/415; C10L 1/02; C12P 7/6409; C12R 1/89

(52) U.S. Cl. CPC C12N 1/12; C07K 14/415; C10L 1/02; C12P 7/6409; C12R 2001/89

(57) ABSTRACT

The present invention provides single-celled photosynthetic organisms that are engineered to express fatty acid transporter in their cytoplasmic membrane and are capable of transporting fatty acids into a culture medium in which they are grown. These engineered organisms can be used to produce large amounts of fatty acids at scale without the need for an energy-intensive disruption step to harvest the fatty acids. The invention also relates to nucleic acid constructs for producing such engineered organisms. The invention further provides methods of culturing such organisms in a medium suitable for growing them in order to produce fatty acids.

### CROSS-REFERENCE TO RELATED APPLICATIONS

This application claims priority to UK patent application GB2205111.4, which was filed on 7 Apr. 2022 and the disclosure of which is hereby incorporated by reference.

### SEQUENCE LISTING

The present specification makes reference to a Sequence Listing, which was submitted electronically as an .xml file name “2023-03-27 P50270WO Sequence Listing” on 6 Apr. 2023. The .xml file was generated on 27 Mar. 2023 and is 40 KB in size. The entire contents of the sequence listing are herein incorporated by reference.

### FIELD OF THE INVENTION

The invention relates to engineered single-celled photosynthetic organisms such as microalgae that can be used to produce large amounts of fatty acids at scale. These organisms may find use in the energy-efficient production of microalgae-derived biofuels.

### BACKGROUND OF THE INVENTION

Microalgae are single-celled photosynthetic organisms that live in waterbodies. They are amongst the most efficient photosynthesisers and can be 400× more efficient than trees. In addition, they are responsible for 50% of global oxygen generation each year. Microalgae cultivation does not require arable land, so a simple pool dug on wasteland would practically suffice.

Since the early 2000s microalgae fuel research has seen a great boom, aided by a particularly vibrant investment environment. Large-scale farms were built, infrastructures were constructed, and even prototype fuels were provided to US Air Force with good effect. However, as the conflicts in the Middle East abated and crude oil price stabilized, the high cost associated with microalgae fuel rendered the technology economically unviable.

The main factor that has prevented the success of large-scale microalgae fuel is the processing cost. Microalgae have evolved for millions of years to be very good at making oil from sunlight and CO₂ and then storing it, but getting the oil out is difficult and expensive. The costs for the energy required to collect the microalgae cells, to remove the water from the cells, to break open the cells, and to separate the useful material from the cell debris, far overshadow the revenue gained from the product, especially when the competition is crude oil (Patel et al. (2020) An Overview of Potential Oleaginous Microorganisms and Their Role in Biodiesel and Omega-3 Fatty Acid-Based Industries. Microorganisms, 8(3), 434). The processing can take up to 70% of the final cost of the fuel. Many major microalgae fuel companies either went under or pivoted into producing products with a much higher retail price, including Algenol, Sapphire Energy, and Solazyme (later TerraVia, part of Corbion N. V., a Dutch food and biochemical company). Other types of advanced biofuels also include non-crop grass farming and subsequent enzymatic conversion or thermal liquefaction to fuel, however its development has not passed experimental phases and its potentials never reached economic feasibility.

Accordingly, a need exists to provide means for producing biofuels that are less energy and resource-intensive and are easily scalable to achieve economic feasibility.

### SUMMARY OF THE INVENTION

The invention is based on the discovery that single-celled photosynthetic organisms can be engineered to express a fatty acid transporter. The inventors have demonstrated that these engineered organisms are capable of secreting fatty acids into the culture medium in which they are grown. This avoids an energy-intensive disruption step to harvest the fatty acids because they can be easily extracted from the medium or the cultured organisms. Using established technologies, the extracted fatty acids can be converted into biofuel through transesterification, decarboxylation or hydrocracking.

In particular, the invention relates to an engineered single-celled photosynthetic organism comprising an exogenous nucleic acid sequence comprising a coding sequence for a fatty acid transporter operationally linked to a promoter sequence. In accordance with the invention, the fatty acid transporter is localised in the cytoplasmic membrane of the organism upon expression.

In some embodiments, the fatty acid transporter is capable of transporting fatty acids with a carbon chain length of C6 to C22 across the cytoplasmic membrane of the organism from inside the cell. In some embodiments, the fatty acid transporter is capable of transporting fatty acids with a carbon chain length of C14 to C22 across the cytoplasmic membrane of the organism from inside the cell.

In some embodiments, the fatty acid transporter is capable of transporting saturated fatty acids across the cytoplasmic membrane of the organism from inside the cell. In some embodiments, the fatty acid transporter is capable of transporting polyunsaturated fatty acids across the cytoplasmic membrane of the organism from inside the cell. In some embodiments, the fatty acid transporter is capable of transporting monounsaturated fatty acids across the cytoplasmic membrane of the organism from inside the cell.

In some embodiments, the fatty acid transporter is capable of transporting fatty acids with a carbon chain length of C16 to C18 across the cytoplasmic membrane of the organism from inside the cell. In some embodiments, the fatty acid transporter is capable of transporting fatty acids with a carbon chain length of C16 and/or C18 across the cytoplasmic membrane of the organism from inside the cell. In some embodiments, the fatty acids with a carbon chain length of C16 and/or C18 are monounsaturated. In some embodiments, the fatty acid transporter is capable of transporting palmitoleic acid and/or oleic acid across the cytoplasmic membrane of the organism from inside the cell.

In some embodiments, the fatty acid transporter is capable of transporting fatty acids with a carbon chain length of C18 across the cytoplasmic membrane of the organism from inside the cell. In some embodiments, the fatty acid transporter is capable of transporting oleic acid across the cytoplasmic membrane of the organism from inside the cell.

In some embodiments, the coding sequence for the fatty acid transporter is derived from the genome of a plant cell or a mammalian cell. In particular embodiments, the fatty acid transporter is derived from the genome of a plant cell. In a specific embodiment, the fatty acid transporter is an ABC transporter. For example, the ABC transporter may be the Arabidopsis thaliana ABCG11 protein (also known as WBC11) or a functional homolog thereof. Alternatively, the ABC transporter may be the Oryza sativa ABCG15 protein or a functional homolog thereof.

In further particular embodiments, the coding sequence for the fatty acid transporter is derived from the genome of a mammalian cell. In a specific embodiment, the fatty acid transporter is an ABC transporter or flippase. For example, the flippase may be the Homo sapiens FATP1 protein or a functional homolog thereof.

The promoter sequence may be derived from the genome of an organism that is different from the genome from which the coding sequence is derived. In some embodiments, the promoter sequence is exogenous to the engineered organism. In other embodiments, the promoter sequence is endogenous to the engineered organism. For example, the promoter may be selected from the group consisting of the cauliflower mosaic virus (CaMV) 35S promoter, a Nitrogen Reductase (NR) promoter, a Photosystem I reaction center Subunit II (PSAD) promoter, and a HSP70A-RBCS2 (AR) promoter.

In some embodiments, the coding sequence is codon-optimised for expression in the engineered organism. In some embodiments, the coding sequence comprises one or more introns.

In some embodiments, the exogenous nucleic acid sequence further comprises a selection marker. In a specific embodiment, the selection marker provides antibiotic resistance.

In some embodiments, the exogenous nucleic acid sequence further comprises a terminator sequence operationally linked to the coding sequence. In some embodiments, the terminator sequence is from the genome of an organism that is different from the genome from which the coding sequence and/or the promoter sequence is/are derived. In some embodiments, the terminator sequence is an exogenous to the engineered organism. In other embodiments, the terminator sequence is endogenous to the engineered organism. In particular embodiments, the terminator sequence encodes an Agrobacterium tumefaciens nopaline synthase (NOS) terminator.

In some embodiments, the single-celled photosynthetic organism is an algae. In some embodiments, the algae is an oleaginous algae. In some embodiments, the single-celled photosynthetic organism is capable of growing in fresh water. In some embodiments, the single-celled photosynthetic organism is capable of growing in salt water. In some embodiments, the single-celled photosynthetic organism is an algae selected from Chlorophyta, Phaeophyta, Rhodophyta, Xanthophyta, Chrysophyta, Bacillariophyta, Cryptophyta, Dinophyta, Euglenophyta, Cyanophyta and Myxophyta. In some embodiments, the single-celled photosynthetic organism is selected from the group consisting of Chlorella, Chlamydomonas, Dunaliella, Haematococcus, Phaeodactylum, Tetraselmis, Isochrysis, Diacronena, Schizochytrium, Thraustochytrium, Nannochloris, Nannochloropsis, Microchloropsis, Porphyridium, Nanofrustulum, Cryptheiconidium, Schenedesmus, Euglena, Auxenochlorella, Botryococcus, Alexandrium, Fistulifera and Nitzschia. In some embodiments, the single-celled photosynthetic organism is selected from the group consisting of Chlorella sorokiniana, Chlorella vulgaris, Chlorella protothecoides, Chlamydomonas reinhardtii, Dunaliella salina, Dunaliella tertiolecta, Dunaliella sp., Haematococcus pluvialis, Phaeodactylum tricornutum, Tetraselmis suecica, Tetraselmis chuii, Isochrysis galbana, Diacronena volkianum, Schizochytrium sp. Thraustochytrium sp., Nannochloris sp. Nannochloropsis sp., Nannochloropsis gaditana, Porphyridium sp., Nanofrustulum sp., Cryptheiconidium cohnii, Scenedesmus sp., Euglena gracilis, Tetraselmis elliptica, Auxenochlorella protothecoides, Botryococcus braunii, Chlorella minutissima, Nannochloropsis salina, Alexandrium sanguinea, Fistulifera solaris, and Nitzschia laevis.

The invention also relates to a nucleic acid comprising a coding sequence for a fatty acid transporter operationally linked to a promoter sequence. In accordance with the invention, the nucleic acid is suitable for inducing expression of the fatty acid transporter in a single-celled photosynthetic organism which does not naturally comprise such a fatty acid transporter in its cytoplasmic membrane (i.e., the coding sequence for the fatty acid transporter is exogenous to single-celled photosynthetic organism). In some embodiments, the fatty acid transporter is capable of transporting fatty acids with a carbon chain length of C6 to C22 across the cytoplasmic membrane of the organism from inside the cell. In some embodiments, the fatty acid transporter is capable of transporting fatty acids with a carbon chain length of C14 to C22 across the cytoplasmic membrane of the organism from inside the cell.

In some embodiments, the fatty acid transporter is capable of transporting saturated fatty acids across the cytoplasmic membrane of the organism from inside the cell. In some embodiments, the fatty acid transporter is capable of transporting polyunsaturated fatty acids across the cytoplasmic membrane of the organism from inside the cell. In some embodiments, the fatty acid transporter is capable of transporting monounsaturated fatty acids across the cytoplasmic membrane of the organism from inside the cell.

In some embodiments, the fatty acid transporter is capable of transporting fatty acids with a carbon chain length of C16 to C18 across the cytoplasmic membrane of the organism from inside the cell. In some embodiments, the fatty acid transporter is capable of transporting fatty acids with a carbon chain length of C16 and/or C18 across the cytoplasmic membrane of the organism from inside the cell. In some embodiments, the fatty acids with a carbon chain length of C16 and/or C18 are monounsaturated. In some embodiments, the fatty acid transporter is capable of transporting palmitoleic acid and/or oleic acid across the cytoplasmic membrane of the organism from inside the cell.

In some embodiments, the fatty acid transporter is capable of transporting fatty acids with a carbon chain length of C18 across the cytoplasmic membrane of the organism from inside the cell. In some embodiments, the fatty acid transporter is capable of transporting oleic acid across the cytoplasmic membrane of the organism from inside the cell.

In some embodiments, the coding sequence for the fatty acid transporter is derived from the genome a plant cell or a mammalian cell. In particular embodiments, the coding sequence for the fatty acid transporter is derived from the genome of a plant cell. In specific embodiments, the coding sequence for the fatty acid transporter encodes an ATP-binding cassette (ABC) transporter. For example, the ABC transporter may be the Arabidopsis thaliana ABCG11 protein (also known as WBC11) or a functional homolog thereof.

Alternatively, the ABC transporter may be the Oryza sativa ABCG15 protein or a functional homolog thereof.

In some embodiments, the coding sequence for the fatty acid transporter is derived from the genome of a mammalian cell. In some embodiments, the coding sequence for the fatty acid transporter encodes an ABC transporter or a flippase. In particular embodiments, the coding sequence for the fatty acid transporter encodes the Homo sapiens flippase FATP1 protein or a functional homolog thereof.

In some embodiments, the promoter sequence is derived from the genome of an organism that is different from the genome from which the coding sequence is derived. In some embodiments, the promoter is selected from the group consisting of the cauliflower mosaic virus (CaMV) 35S promoter, a Nitrogen Reductase (NR) promoter, a Photosystem I reaction center Subunit II (PSAD) promoter, and a HSP70A-RBCS2 (AR) promoter. In some embodiments, the promoter is derived from a different organism to the fatty acid transporter.

In some embodiments, the coding sequence for the fatty acid transporter is codon-optimised. In some embodiments, the coding sequence further comprises one or more introns.

In some embodiments, the nucleic acid further comprising a selection marker. In some embodiments, the selection marker provides antibiotic resistance.

In some embodiments, the nucleic acid further comprises a terminator sequence operationally linked to the coding sequence. In some embodiments, the terminator sequence is derived from the genome of an organism that is different from the genome from which the coding sequence and/or the promoter sequence is/are derived. In some embodiments, the terminator sequence is derived from the genome of an organism that is different from the genome of the organism(s) from which the coding sequence is derived. In some embodiments, the terminator sequence is derived from the genome of an organism that is different from the genome of the organism(s) from which the promoter sequence is derived. In some embodiments, the terminator sequence is derived from the genome of an organism that is different from the genome of the organism(s) from which the coding sequence and the promoter sequence are derived. In some embodiments, the terminator is the Agrobacterium tumefaciens nopaline synthase (NOS) terminator.

The invention also relates to a culture comprising a engineered single-celled photosynthetic organism of the invention.

The invention also relates to a method for producing fatty acids comprising culturing an engineered single-celled photosynthetic organism of the invention in a medium suitable for growing the organism. In some embodiments, culturing is continuous at a steady state. In some embodiments, the method further comprises a step of separating the medium from the organism. In some embodiments, the step of separating comprises sedimentation or filtration. In some embodiments, sedimentation involves centrifugation. In some embodiments, sedimentation involves incubating the medium without agitation for a period of time. In some embodiments, the method further comprises a step of extracting the fatty acids from the organism-free medium obtained by sedimentation or filtration using a liquid-liquid extraction process. In some embodiments, the method further comprises spooning droplets comprising the fatty acids from the surface of the organism-free culture medium obtained by sedimentation or filtration to extract the fatty acids. In some embodiments, the method further comprises processing the extracted fatty acids by transesterification. In some embodiments, the method further comprises processing the extracted fatty acids by decarboxylation. In some embodiments, the method further comprises processing the extracted fatty acids by hydrocracking.

In some embodiments, a method for producing fatty acids in accordance with the invention produces at least 1 μg fatty acid per litre of culture medium per day. In some embodiments, a method for producing fatty acids in accordance with the invention produces at least 10 μg fatty acid per litre of culture medium per day. In some embodiments, a method for producing fatty acids in accordance with the invention produces at least 100 μg fatty acid per litre of culture medium per day. In some embodiments, a method for producing fatty acids in accordance with the invention produces at least 1 g fatty acid per litre of culture medium per day.

The invention also relates to a culture medium that was inoculated with a engineered single-celled organism of the invention and incubated for a period of time sufficient to yield at least 1 μg fatty acid per litre of culture medium. In some embodiments, the culture medium was incubated for a period of time sufficient to yield at least 10 μg fatty acid per litre of culture medium. In some embodiments, the culture medium was incubated for a period of time sufficient to yield at least 100 μg fatty acid per litre of culture medium. In some embodiments, the culture medium was incubated for a period of time sufficient to yield at least 1 g fatty acid per litre of culture medium. In some embodiments, the culture medium is free of the organism used for inoculation.

The invention also relates to a method of extracting fatty acids from a single-celled photosynthetic organism of the invention, wherein the method comprises (i) providing a culture medium that was inoculated with a engineered single-celled organism of the invention and incubated for a period of time sufficient to yield at least 1 μg fatty acid per litre of culture medium; and (ii) extracting the fatty acids. In some embodiments, the step of extracting comprises a liquid-liquid extraction process. In some embodiments, the step of extracting comprises spooning droplets comprising the fatty acids from the surface of the culture medium.

The invention also relates to a method of producing a biofuel, comprising providing a fatty acid obtained by one of the methods described in the preceding paragraphs; and processing the fatty acid by transesterification, decarboxylation or hydrocracking. The invention also relates to a biofuel obtained by such method.

### BRIEF DESCRIPTION OF THE DRAWINGS

Embodiments of the invention are described, by way of example, with reference to the following drawings, in which:

FIG. 1 provides phylogenetic trees identifying homologues to representative fatty acid transporters suitable for use with the invention. FIG. 1a provides representative predicted functional homologues to the Arabidopsis thaliana ABC transporter ABCG11.

FIG. 1b provides representative functional homologues to the Oryza sativa subspecies japonica ABC transporter ABCG15. The shown homologues typically have sequence identities of at least 80% relative to ABCG11 and ABCG15, respectively.

FIG. 2 provides a phylogenetic tree identifying representative functional homologues of the human flippase FATP1 (NP_940982.1) suitable for use with the invention. Shown homologues within the phyla Animalia and Fungus have sequence identities of at least 90% and at least 25%, respectively, relative to human FATP1.

FIG. 3 schematically illustrates exemplary nucleic acids comprising a coding sequence for a fatty acid transporter suitable for use with the invention. The nucleic acids comprise a selection marker (aadA), which is operationally linked to β-tubulin promoter and terminator sequences. The nucleic acid (A) does not contain a coding sequence for a fatty acid transporter. It is used as a mock control. Nucleic acids (B)-(G) include a coding sequence for a fatty acid transporter (B, E: FATP1; C, F: ABCG11; D, G: ABCG15). The coding sequence for the fatty acid transporter is located at a 3′ position relative to selection marker. It is operationally linked to a promoter sequence (CaMV 35S) at the 5′ end and a terminator sequence (NOS) at the 3′ end. The nucleic acids (E)-(G) additionally include a sequence encoding a fluorescent marker protein (Clover) between the coding sequence for the fatty acid transporter and the terminator sequence. The fluorescent marker protein is expressed with the fatty acid transporter as a fusion protein upon successful transformation of a target organism of interest. The locations of introns within the coding sequence of the fatty acid transporter and the sequence encoding a fluorescent marker protein are also shown.

FIG. 4 provides PCR confirmation of insertion of an exogenous nucleic acid sequence comprising a coding sequence for a fatty acid transporter in the genome of C. reinhardtii, a single-celled organism suitable for use with the invention. FIG. 4 represents the gel electrophoresis results of the representative clones. A 567 bp fragment of the selection marker sequence (aadA) was PCR amplified from genomic DNA obtained from transformed C. reinhardtii. Untransformed wild-type cells served as a control. Lane 1, wild-type (not transformed); 2, cells transformed with antibiotic cassette only; 3-4, FATP1; 5-6, FATP1-clover; 7-8, ABCG11; 9-10, ABCG11-clover; 11-12, ABCG15; 13-14, ABCG15-clover. Detailed genetic constructs are shown in FIG. 3.

FIG. 5 provides immunofluorescence assay images of C. reinhardtii transformed with exogenous fatty acid transporters fused to a clover fluorescent protein. FIG. 5a shows C. reinhardtii transformed with a nucleic acid encoding an antibiotic selection marker only (mock control). Only background fluorescence is visible in FIG. 5a. FIG. 5b shows C. reinhardtii transformed with a nucleic acid encoding an ABCG15-clover fusion protein. Clusters of fluorescent cells are clearly visible in FIG. 5b, with the fluorescent staining concentrated in the cytoplasmic membrane (scale bar: 20 μm).

FIG. 6 illustrates the results of the BODIPY staining assay, showing extracellular oil deposits from C. reinhardtii transformed with a nucleic acid encoding an exogenous fatty acid transporter in accordance with the invention. BODIPY is a stain for oil and other nonpolar lipids. Panels a, c, e and g are brightfield images. Panels b, d, f and h are fluorescence images of the same field of view. Panels a and b show C. reinhardtii transformed with a nucleic acid encoding an antibiotic selection marker only (mock control). No extracellular oil depots are visible in panel b. Panels c and d show C. reinhardtii transformed with a nucleic acid encoding the fatty acid transporter FATP1. Panels e and f show C. reinhardtii transformed with a nucleic acid encoding the fatty acid transporter ABCG11. Panels g and h show C. reinhardtii transformed with a nucleic acid encoding the fatty acid transporter ABCG15. Extracellular oil deposits are clearly visible panels d, f and h (see arrows).

FIG. 7 illustrates an exemplary liquid chromatography-mass spectrometry (LC-MS) analysis of fatty acids present in culture media comprising C. reinhardtii cells. Sample A corresponds to a clone of C. reinhardtii expressing a fatty acid transporter after having been transformed with an expression cassette comprising the human gene SLC27A1 (encoding FATP1) and an antibiotic resistance gene. Sample B corresponds to a clone transformed with only the antibiotic resistance gene (i.e., non-SLC27A1-expressing; mock control). FIG. 7a shows the detection of palmitoleic acid (C16:1) in culture medium collected from these C. reinhardtii clones. FIG. 7b shows the detection of oleic acid (C18:1) in culture medium collected from these C. reinhardtii clones. Data are visualised by box and whisker plots. The SLC27A1-expressing C. reinhardtii clone in sample A secreted significantly higher amounts of the monounsaturated fatty acids C16:1 and C18:1 into the culture medium than the non-SLC27A1-expressing clone in sample B.

FIG. 8 illustrates successful expression of the fatty acid ABC transporter ABCG11 in the microalga C. sorokiniana (strain SAG 211-32). A Western blot of C. sorokiniana lysates was probed with an anti-His-tag antibody. Lane ‘L’ shows a molecular weight ladder.

Lane ‘1’ comprises a lysate from wild-type cells which do not express the ABCG11 transporter (negative control). Lane ‘2’ comprises a lysate from ABCG11-expressing cells. Lane ‘3’ comprises a His-tagged protein used as a positive control. ABCG11 transgene expression is shown in lane 2 by a protein band at ˜118 kDa, corresponding to ABCG11+6His-tag.

FIG. 9 illustrates transformation of the microalga C. sorokiniana (strain SAG 211-32) with exogenous nucleic acids encoding various fatty acid transporters. Cells were transformed with nucleic acids encoding a human flippase (FATP1) or plant ABC transporters from Oryza sativa (ABCG15) or Arabidopsis thaliana (ABCG11), each fused to a His tag for ease of detection. To select transformants, cells were plated onto agar-TAP plates in the presence of G418. An illustrative set of plates is shown in FIG. 9a. Transformants were picked and grown in culture medium in the presence of G418, lysed and screened for fatty acid transporter expression by Western blot. Western blots were probed with an anti-His-tag antibody to detect transgene expression. FIGS. 9b and 9c are representative examples of Western blots performed as part of the screen. FIG. 9b shows His-tag detection in lysates from cells transformed with transgenes encoding FATP1 (lanes 1-8) and ABCG11 (lanes 9-11), respectively. FIG. 9c shows His-tag detection in lysates from cells transformed with transgenes encoding ABCG11 (lanes 1-4) and ABCG15 (lanes 5-11), respectively. Not all clones successfully expressed the transgene. Samples in which successful transgene expression was detected are indicated by black rectangles. Lane ‘L’ shows a molecular weight ladder. In both panels, lane 12 comprises lysates from wild-type cells (negative control), and lane 13 comprises a His-tagged protein (positive control).

FIG. 10 illustrates fatty acid secretion into the culture medium by C. sorokiniana cells expressing various fatty acid transporters. FIG. 10a shows that cells were successfully transformed with exogenous nucleic acids encoding a human flippase FATP1, the ABC transporter ABCG15 from Oryza sativa or the ABC transporter ABCG11 from Arabidopsis thaliana (ABCG11), respectively. Using an anti-His tag antibody, bands corresponding in their molecular weight to FAPT1 (lanes 1-4), ABCG11 (lanes 5-8) and ABCG15 (lanes 9-12), respectively, were detected. Lane 13 comprises a His-tagged protein (positive control). Lane ‘L’ shows a molecular weight ladder.

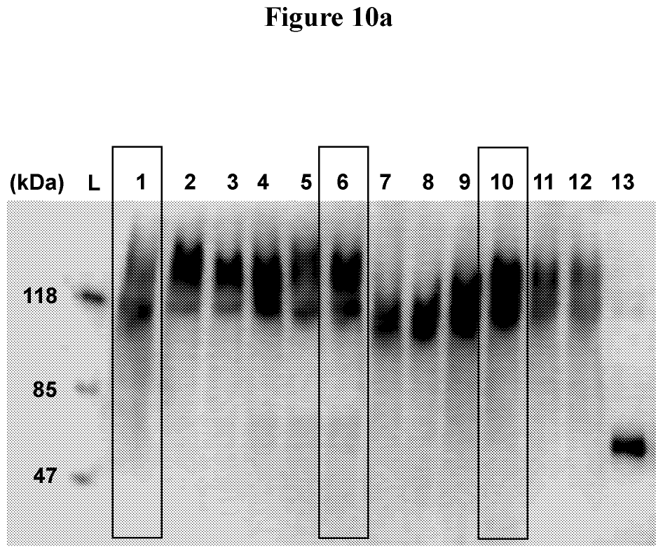

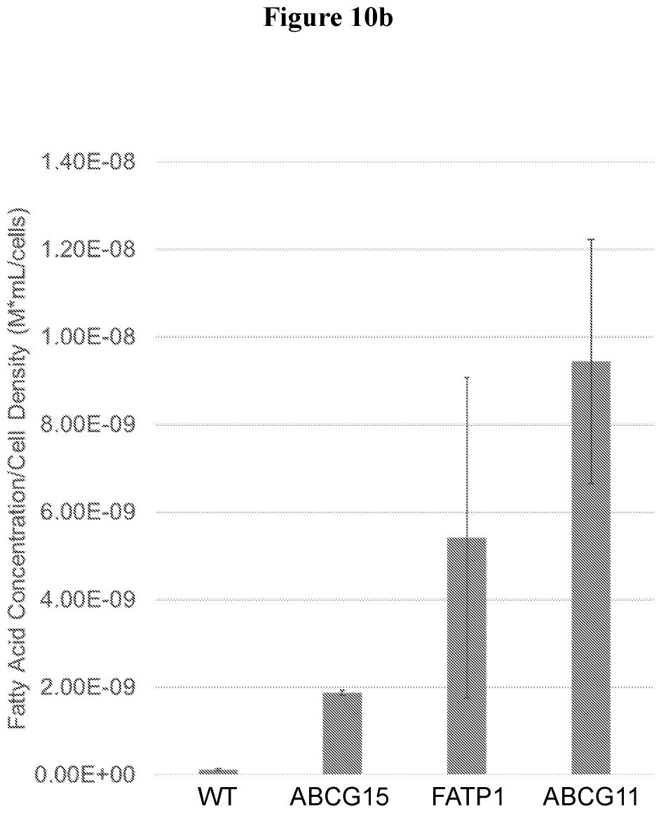

Liquid cultures (in TAP medium) were prepared with one of the transformants of each set (ABCG15, FATP1 and ABCG11). The selected transformants are indicated by a black rectangle in FIG. 10a. A culture with a corresponding wild-type cell was incubated under the same conditions. The culture media were sampled for the presence of free fatty acids normalised by cell density for ease of comparison. FIG. 10b shows normalised free fatty acid concentrations in the culture media after 48 hours of incubation with cells expressing the indicated atty acid transporters. All fatty acid transporter-expressing cells secreted significantly higher amounts of fatty acids into the culture medium than wild-type (WT) control cells.

### Definitions

In order for the present invention to be more readily understood, certain terms are first defined below. Additional definitions for the following terms and other terms are set forth throughout the specification.

As used in this specification and the appended claims, the singular forms “a”, “an” and “the” include plural referents unless the context clearly dictates otherwise.

Unless specifically stated or obvious from context, as used herein, the term “or” is understood to be inclusive and covers both “or” and “and”.

As used herein, the term “single-celled photosynthetic organism” means any single-celled organism that is able to transform light energy into chemical energy. Examples of photosynthetic organisms include cyanobacteria, plants and algae.

As used herein, the term “fatty acid transporter” means any protein capable of transporting one or more fatty acids across a lipid bilayer membrane.

As used herein, the terms “transports” and “transporting” mean moving a molecule from one side of a lipid bilayer membrane to the other side.

The terms “secretes” and “secreting” is used herein interchangeably with the term “transports” and “transporting”.

As used herein, the term “operationally-linked” means that the sequence performs its function on the coding sequence with which it is associated. For instance, an operationally-linked promoter is capable of driving expression of the coding sequence to which it is operationally-linked. An operationally-linked terminator is capable of terminating transcription of the coding sequence to which it is operationally-linked.

As used herein, the term “target organism” refers to a single-celled photosynthetic organism suitable for use with the invention that may be transformed with a nucleic acid comprising a coding sequence for a fatty acid transporter as described herein.

As used herein, the term “ATP-binding cassette transporter” (ABC transporter) refers to any member of the superfamily of ABC transport systems. In particular embodiments, the term is used herein to describe eukaryotic ABC transporters. Typically, ABC transporters couple the hydrolysis of ATP to the translocation of a substrate across a biological membrane.

As used herein, the term “flippase” refers to a protein falling within the subfamily of P-type ATPases. Flippases typically act as transmembrane lipid transporter proteins.

As used herein, the term “functional homologue” refers to a homologous protein that is capable of the same function as the reference protein.

All technical and scientific terms used herein have the same meaning as commonly understood by one of ordinary skill in the art to which this application belongs and as commonly used in the art to which this application belongs. The publications and other reference materials referenced herein to describe the background of the invention and to provide additional detail regarding its practice are hereby incorporated by reference.

### DETAILED DESCRIPTION OF THE INVENTION

The invention relates to single-celled photosynthetic organisms that are engineered to express fatty acid transporters in their cytoplasmic membrane and are capable of transporting fatty acids into a culture medium in which they are grown.

In particular, the invention relates to an engineered single-celled photosynthetic organism comprising an exogenous nucleic acid sequence comprising a coding sequence for a fatty acid transporter operationally linked to a promoter sequence. An engineered organism of the invention comprising the exogenous nucleic acid is capable of transporting fatty acids across its cytoplasmic membrane in an amount greater than an otherwise identical organism cultured under identical conditions, but lacking the exogenous nucleic acid. Typically, a culture medium comprising an engineered single-celled photosynthetic organism comprising an exogenous nucleic acid in accordance with the invention comprises an at least 3-fold higher concentration of fatty acids than a corresponding culture medium comprising an otherwise identical organism cultured under identical conditions, but lacking the exogenous nucleic acid. In some embodiments, the fatty acid concentration in a culture medium comprising an engineered single-celled photosynthetic organism comprising an exogenous nucleic acid in accordance with the invention is at least 5-fold higher, at least 10-fold higher, at least 20-fold higher, at least 30-fold higher, at least 40-fold higher, at least 50-fold higher, at least 60-fold higher, at least 70-fold higher, at least 80-fold higher, at least 90-fold higher, or at least 100-fold higher than a corresponding culture medium comprising an otherwise identical organism cultured under identical conditions, but lacking the exogenous nucleic acid.

The invention also relates to a culture medium conditioned by an engineered single-celled photosynthetic organism in accordance with the invention, wherein the culture medium is enriched for monounsaturated fatty acids having a carbon chain length of C16 to C18 relative to a culture medium conditioned by a corresponding wild-type single-celled photosynthetic organism under the same conditions. In some embodiments, a culture medium conditioned by an engineered single-celled photosynthetic organism in accordance with the invention comprises at least about 5-fold (e.g., at least about 10-fold) more palmitoleic acid (C16:1) than a culture medium conditioned by a corresponding wild-type single-celled photosynthetic organism under the same conditions. In some embodiments, a culture medium conditioned by an engineered single-celled photosynthetic organism in accordance with the invention comprises at least about 1.5-fold (e.g., at least about 3-fold or at least about 5-fold) more oleic acid (C18:1) than a culture medium conditioned by a corresponding wild-type single-celled photosynthetic organism under the same conditions.

The invention also relates to a cell-free fatty acid composition secreted by an engineered single-celled photosynthetic organism in accordance with the invention, wherein the composition is enriched for monounsaturated fatty acids having a carbon chain length of C16 to C18 relative to a composition secreted by a corresponding wild-type single-celled photosynthetic organism under the same conditions. In some embodiments, a cell-free fatty acid composition secreted by an engineered single-celled photosynthetic organism in accordance with the invention comprises at least about 5-fold (e.g., at least about 10-fold) more palmitoleic acid (C16:1) than a composition secreted by a corresponding wild-type single-celled photosynthetic organism under the same conditions. In some embodiments, a cell-free fatty acid composition secreted by an engineered single-celled photosynthetic organism in accordance with the invention comprises at least about 1.5-fold (e.g., at least about 3-fold or at least about 5-fold) more oleic acid (C18:1) than a composition secreted by a corresponding wild-type single-celled photosynthetic organism under the same conditions.

The engineered single-celled photosynthetic organisms of the invention can be used to produce large amounts of fatty acids at scale without the need for an energy-intensive disruption step to harvest the fatty acids. The invention also relates to nucleic acid constructs for producing such engineered organisms. The invention further provides methods of culturing such organisms in a medium suitable for growing them in order to produce fatty acids.

### Fatty Acids

Fatty acids are composed of a carboxylic acid with an aliphatic chain, which is either saturated or unsaturated. They are a primary metabolite used by cells for energy storage. Naturally occurring fatty acids typically have an unbranched aliphatic chain of an even number of carbon atoms, from 4 to 28 (C4 to C28). Fatty acids can be the major component of the lipids (up to 70 wt %) in some microalgae species. Such microalgae species may be particularly suitable for use with this invention.

Fatty acids with a carbon chain length of C6 to C22 are of particular interest for industrial applications such as biofuel production.

### Fatty Acid Transporters

A biological membrane is composed of a lipid bilayer, with hydrophobic sides facing each other while the polar sides face either side. Lipid bilayer membranes are semi-permeable: small molecules can diffuse through the membrane, while the increasing size of a molecule is inversely proportional by orders of magnitude to the rate of diffusion through the lipid bilayer membrane. Lipid bilayer membranes are virtually impermeable to large molecules. Cells possess dedicated mechanisms for the transport of large molecules, such as fatty acids, across lipid bilayer membranes.

Certain transporters are capable of transporting fatty acids of varying lengths across lipid bilayer membranes. Fatty acid transporters may be capable of transporting fatty acids of any length across the cytoplasmic membrane of the organism from inside the cell. This may include fatty acids with a carbon chain length of C6 to C22. Certain fatty acid transporters may preferentially transport fatty acids of specific lengths. For example, typical substrates for naturally occurring fatty acid transporters are fatty acids with a carbon chain length of C14 to C22. Fatty acid transporters are particularly effective in transporting fatty acids with a carbon chain length of about C18 across the cytoplasmic membrane and other lipid bilayer membranes. For instance, fatty acid transporters disclosed herein are particularly effective in transporting fatty acids with a carbon chain length of C16 to C18 across the cytoplasmic membrane and other lipid bilayer membranes. These fatty acid transporter are also particularly effective in transporting monounsaturated fatty acids (e.g., C16:1 and C18:1) across the cytoplasmic membrane and other lipid bilayer membranes.

Accordingly, in some embodiments, a fatty acid transporter suitable for use with the invention is capable of transporting fatty acids with a carbon chain length of C6 to C22. In some embodiments, a fatty acid transporter suitable for use with the invention is capable of transporting fatty acids with a carbon chain length of C14 to C22 (e.g., C16 to C18) across the cytoplasmic membrane of an organism from inside the cell. In particular embodiments, a fatty acid transporter suitable for use with the invention is capable of transporting fatty acids with a carbon chain length of about C16 and/or C18 across the cytoplasmic membrane of an organism from inside the cell. In some embodiments, the fatty acids with a carbon chain length of C16 and/or C18 are monounsaturated. In some embodiments, a fatty acid transporter for use with the present invention is capable of transporting palmitoleic acid and/or oleic acid.

Fatty acid transporters suitable for use with the invention are typically derived from a eukaryotic cell. In particular, the inventors have identified plant and mammalian fatty acid transporters and have demonstrated successful secretion of fatty acids from single-celled photosynthetic organisms that were engineered to express these fatty acid transporters.

Homologues have been identified for many fatty acid transporters. Without wishing to be bound by theory, functional homologues of fatty acid transporters are expected to transport fatty acids across lipid bilayer membranes with similar specificity for fatty acids of a certain length (e.g., fatty acids with a carbon chain length of C6 to C22). Functional homologues of the fatty acid transporters described herein may be used in place of the exemplified fatty acid transporters described herein.

While the invention is described by reference to representative fatty acid transporters, it should not be construed to be limited to the particular fatty acid transporters described herein.

### ATP-Binding Cassette (ABC) Transporters

ATP-binding cassette (ABC) transporters constitute a ubiquitous superfamily of integral membrane proteins that are responsible for the ATP-powered transport of many substrates across membranes. This superfamily is conserved from prokaryotes to eukaryotes only in structure, rather than sequences. The protein forms a channel in the lipid bilayer and regulates the influx or efflux of specific molecules. One ATP molecule is hydrolyzed per transported cargo (e.g., a single substrate molecule such as fatty acid).

Across the members of the superfamily, substrate specificity is varied. ABC transporters have been identified translocating a range of substrates, including lipids, retinoic acid derivatives, bile acid, iron, nucleosides and peptides. Some ABC transporters are thought to have roles in the transport of lipids and lipid-related compounds. For example, almost half of the 48 identified human ABC transporter proteins are thought to facilitate translocation of lipids or lipid-related compounds. In some embodiments, any ABC transporters that facilitate the translocation of lipids or lipid-related compounds may be used in place of the fatty acid transporters described herein. More typically, the ABC transporters for use with the invention are capable to transport fatty acids across the cytoplasmic membrane of a target organism, in particular those fatty acids that are primary metabolites used by the target organism for energy storage (e.g., fatty acids with a carbon chain length of C14 to C22).

### Flippases

Flippases are a sub-family of P-type ATPases characterized as P4-ATPases. Flippases typically flip phospholipids across cell membranes and play a role in a myriad of processes including vesicle budding and trafficking, cell signaling, blood coagulation, apoptosis, bile, cholesterol homeostasis and neuronal cell survival. Different flippases have different targets and specificities, e.g., they may preferentially transport fatty acids of a specified chain length and a specified degree of saturation across lipid bilayer membranes. For example, some flippases transport phosphatidylethanolamine across lipid bilayer membranes whereas other flippases are specific for phosphatidylcholine.

In a typical embodiment, a flippase for use with the invention is a eukaryotic flippase. For example, a suitable flippase may be derived from a plant cell or a mammalian cell. In particular embodiments, a flippase derived from a mammalian cell may be a Homo sapiens flippase.

### Substrate Specificity

The inventors have found that fatty acid transporters for use with the invention are capable of preferentially transporting fatty acids of a specified chain length and a specified degree of saturation across the cytoplasmic membrane of an organism from inside the cell. For example, the inventors observed that fatty acid transporters (e.g., flippases) preferentially transport monounsaturated fatty acids with C16 and C18 carbon chain lengths (C16:1 and C18:1) across the cytoplasmic membrane.

The term “preferentially transport” as used in this context refers to the substrate specificity of the fatty acid transporter. For example, the fatty acid transporter may transport at least 1.5- to 2-fold more (e.g., at least 3-fold more, at least 5-fold more or at least 10-fold more) of one fatty acid in comparison to one or more other fatty acid. In some embodiments, a fatty acid transporter for use with the invention (e.g., a Homo sapiens flippase) transports at least 1.5- to 5-fold more monounsaturated fatty acids with C16 and C18 carbon chain lengths (C16:1 and C18:1) than saturated or polyunsaturated fatty acids (e.g., C14:0, C16:0, C18:0, C18:2) across the cytoplasmic membrane.

In some embodiments, the substrate specificity of fatty acid transporters of the invention may be exploited by further engineering the organism to overproduce fatty acid(s) that are preferentially transported by the fatty acid transporter. For example, metabolic engineering may be used to provide an engineered single-celled photosynthetic organism that overproduces fatty acids of a specified chain length and a specified degree of saturation (as determined in comparison to a corresponding single-celled photosynthetic organism that was not subjected to metabolic engineering).

In particular embodiments, the single-celled photosynthetic organism is metabolically engineered to overproduce monounsaturated fatty acids with C16 and/or C18 carbon chain lengths (e.g., palmitoleic acid and/or oleic acid). As shown herein, flippases (e.g., a Homo sapiens flippase) are capable of preferentially transporting monounsaturated fatty acids with C16 and/or C18 carbon chain lengths across the cytoplasmic membrane of single-celled photosynthetic organism such as microalgae.

In some embodiments, a fatty acid transporter for use with the invention has minimal transport activity for fatty acids with carbon chain lengths less than C14. In some embodiments, a fatty acid transporter for use with the invention has minimal transport activity for fatty acids with carbon chain lengths greater than C22. In particular embodiments, a fatty acid transporter for use with the invention has minimal transport activity for fatty acids with carbon chain lengths less than C14 and greater than C22. The term “minimal” in this context means that less than 1% of the fatty acids transported from inside the cell across the plasma membrane have a carbon chain length of less than C14 and/or greater than C22.

In some embodiments, transport activity is assessed by determining fatty acid transport across a lipid bilayer of an artificial phospholipid vesicle comprising a fatty acid transporter of interest. Such a vesicle may be incubated with a fatty acid, and the appearance of the fatty acid in the internal aqueous phase of the vesicle is monitored (see, e.g., Glatz et al., Physiol Rev. 2010; 90(1): 367-417, which is incorporated herewith by reference).

### Exemplary Amino Acid Sequences Encoding Acid Transporter Proteins

Exemplary amino acid sequences of fatty acid transporter proteins suitable for use with the invention are provided in Table 1.

TABLE 1
Exemplary fatty acid
transporter amino acid sequences

| | SEQ ID | |
|---|---|---|
| NAME | NO: | SEQUENCE |
| Arabidopsis | 1 | MEIEASRQQTTVPVSVGGGNFPVGGLSPLSEAIWREK |
| thaliana ABCG11 | | APTEFVGDVSARLTWQDLTVMVTMGDGETQNVLEG |
| protein | | LTGYAEPGSLTALMGPSGSGKSTMLDALASRLAANAF |
| | | LSGTVLLNGRKTKLSFGTAAYVTQDDNLIGTLTVRETI |
| | | WYSARVRLPDKMLRSEKRALVERTIIEMGLQDCADT |
| | | VIGNWHLRGISGGEKRRVSIALEILMRPRLLFLDEPTS |
| | | GLDSASAFFVTQTLRALSRDGRTVIASIHQPSSEVFELF |
| | | DRLYLLSGGKTVYFGQASDAYEFFAQAGFPCPALRNP |
| | | SDHFLRCINSDFDKVRATLKGSMKLRFEASDDPLEKIT |
| | | TAEAIRLLVDYYHTSDYYYTAKAKVEEISQFKGTILDS |
| | | GGSQASFLLQTYTLTKRSFINMSRDFGYYWLRLLIYIL |
| | | VTVCIGTIYLNVGTSYSAILARGSCASFVFGFVTFMSIG |
| | | GFPSFVEDMKVFQRERLNGHYGVAAFVIANTLSATPF |
| | | LIMITFISGTICYFMVGLHPGFTHYLFFVLCLYASVTVV |
| | | ESLMMAIASIVPNFLMGIIIGAGIQGIFMLVSGFFRLPN |
| | | DIPKPFWRYPMSYISFHFWALQGQYQNDLRGLTFDSQ |
| | | GSAFKIPGEYVLENVFQIDLHRSKWINLSVILSMIIIYRII |
| | | FFIMIKTNEDVTPWVRGYIARRRMKQKNGTQNTTVAP |
| | | DGLTQSPSLRNYIATRTDGARRW |
| Oryza sativa | 2 | MCLLEEMMEISSNEEMMEMAIVEQLPPSSHHLNGGSV |
| ABCG15 protein | | EVDMEEDHVWPTKDGPLPIFLKFENVEYKVKLTPKNP |
| | | LTAARVAFASHKSTEDQGSCKHILKGVGGSVDPGEIL |
| | | ALMGPSGSGKTTLLKILGGRLSGGVRGQITYNDTPYSP |
| | | CLKRRIGFVTQDDVLFPQLTVEETLVFAAFLRLPARMS |
| | | KQQKRDRVDAIITELNLERCRHTKIGGAFVRGVSGGE |
| | | RKRTSIGYEILVDPSLLLLDEPTSGLDSTSAAKLLVVLR |
| | | RLARSAARRTVITTIHQPSSRMFHMFDKLLLVAEGHAI |
| | | YHGGARGCMRHFAALGFSPGIAMNPAEFLLDLATGN |
| | | LDGISSPASLLLPSAAAASPDSPEFRSHVIKYLQARHRA |
| | | AGEEEAAAAAAREGGGGGGAGRDEAAKQLRMAVR |
| | | MRKDRRGGIGWLEQFTVLSRRTFRERAADYLDKMRL |
| | | AQSVGVALLLGLLWWKSQTSNEAQLRDQVGLIFYICI |
| | | FWTSSSLFGSVYVFPFEKLYLVKERKADMYRLSAYYA |
| | | SSTVCDAVPHVVYPVLFTAILYFMADLRRTVPCFCLT |
| | | LLATLLIVLTSQGTGELLGAAILSVKRAGVMASLVLM |
| | | LFLLTGGYYVQHIPKFIRWLKYVSFMHYGFNLLLKAQ |
| | | YHGHLTYNCGSRGGCQRLQSSPSFGTVDLDGGMREV |
| | | WILLAMAVAYRLLAYLCLRKRISLMPL |
| Homo sapiens | 3 | MRAPGAGAASVVSLALLWLLGLPWTWSAAAALGVY |
| FATP1 protein | | VGSGGWRFLRIVCKTARRDLFGLSVLIRVRLELRRHQ |
| | | RAGHTIPRIFQAVVQRQPERLALVDAGTGECWTFAQL |
| | | DAYSNAVANLFRQLGFAPGDVVAIFLEGRPEFVGLWL |
| | | GLAKAGMEAALLNVNLRREPLAFCLGTSGAKALIFGG |
| | | EMVAAVAEVSGHLGKSLIKFCSGDLGPEGILPDTHLL |
| | | DPLLKEASTAPLAQIPSKGMDDRLFYIYTSGTTGLPKA |
| | | AIVVHSRYYRMAAFGHHAYRMQAADVLYDCLPLYH |
| | | SAGNIIGVGQCLIYGLTVVLRKKFSASRFWDDCIKYNC |
| | | TVVQYIGEICRYLLKQPVREAERRHRVRLAVGNGLRP |
| | | AIWEEFTERFGVRQIGEFYGATECNCSIANMDGKVGS |
| | | CGFNSRILPHVYPIRLVKVNEDTMELLRDAQGLCIPCQ |
| | | AGEPGLLVGQINQQDPLRRFDGYVSESATSKKIAHSVF |
| | | SKGDSAYLSGDVLVMDELGYMYFRDRSGDTFRWRG |
| | | ENVSTTEVEGVLSRLLGQTDVAVYGVAVPGVEGKAG |
| | | MAAVADPHSLLDPNAIYQELQKVLAPYARPIFLRLLP |
| | | QVDTTGTFKIQKTRLQREGFDPRQTSDRLFFLDLKQG |
| | | HYLPLNEAVYTRICSGAFAL |

In some embodiments, the invention relates to an engineered single-celled photosynthetic organism recombinantly engineered to be capable of expressing a fatty acid transporter having the amino acid sequence of SEQ ID NO: 1, or a functional homologue thereof. In some embodiments, the invention relates to an engineered single-celled photosynthetic organism recombinantly engineered to be capable of expressing a fatty acid transporter having the amino acid sequence of SEQ ID NO: 2, or a functional homologue thereof. In some embodiments, the invention relates to an engineered single-celled photosynthetic organism recombinantly engineered to be capable of expressing a fatty acid transporter having the amino acid sequence of SEQ ID NO: 3, or a functional homologue thereof.

In some embodiments, the amino acid sequence of a functional homologue of a fatty acid transporter of the invention is at least 25%, 30%, 35%, 40%, 45%, 50%, 55%, 60%, 65%, 70%, 75%, 80%, 85%, 90%, or 95% identical to the amino acid sequence of SEQ ID NO: 1. In some embodiments, the amino acid sequence of a functional homologue of a fatty acid transporter of the invention is at least 25% identical to the amino acid sequence of SEQ ID NO: 1. In some embodiments, the amino acid sequence of a functional homologue of a fatty acid transporter of the invention is at least 80% identical to the amino acid sequence of SEQ ID NO: 1. In some embodiments, the amino acid sequence of a functional homologue of a fatty acid transporter of the invention is at least 90% identical to the amino acid sequence of SEQ ID NO: 1.

In some embodiments, the amino acid sequence of a functional homologue of a fatty acid transporter of the invention is at least 75%, 80%, 85%, 90%, or 95% identical to the amino acid sequence of SEQ ID NO: 2. In some embodiments, the amino acid sequence of a functional homologue of a fatty acid transporter of the invention is at least 80% identical to the amino acid sequence of SEQ ID NO: 2. In some embodiments, the amino acid sequence of a functional homologue of a fatty acid transporter of the invention is at least 90% identical to the amino acid sequence of SEQ ID NO: 2.

In some embodiments, the amino acid sequence of a functional homologue of a fatty acid transporter of the invention is at least 75%, 80%, 85%, 90%, or 95% identical to the amino acid sequence of SEQ ID NO: 3. In some embodiments, the amino acid sequence of a functional homologue of a fatty acid transporter of the invention is at least 80% identical to the amino acid sequence of SEQ ID NO: 3. In some embodiments, the amino acid sequence of a functional homologue of a fatty acid transporter of the invention is at least 90% identical to the amino acid sequence of SEQ ID NO: 3.

In some embodiments, the amino acid sequence of a fatty acid transporter in accordance with the invention is at least 90%, 95%, 96%, 97%, 98%, or 99% identical to the amino acid sequence of SEQ ID NO: 1. In some embodiments, the amino acid sequence of a fatty acid transporter in accordance with the invention is at least 90%, 95%, 96%, 97%, 98%, or 99% identical to the amino acid sequence of SEQ ID NO: 2. In some embodiments, the amino acid sequence of a fatty acid transporter in accordance with the invention is at least 90%, 95%, 96%, 97%, 98%, or 99% identical to the amino acid sequence of SEQ ID NO: 3.

In some embodiments, a fatty acid transporter in accordance with the invention is capable of preferentially transporting monounsaturated fatty acids with C16 and C18 carbon chain lengths (e.g., palmitoleic acid and/or oleic acid) over a lipid bilayer membrane and has an amino acid sequence that is at least 75%, 80%, 85%, 90%, 95%, 96%, 97%, 98%, or 99% identical to the amino acid sequence of SEQ ID NO: 1. In some embodiments, a fatty acid transporter in accordance with the invention is capable of preferentially transporting monounsaturated fatty acids with C16 and C18 carbon chain lengths (e.g., palmitoleic acid and/or oleic acid) over a lipid bilayer membrane and has an amino acid sequence that is at least 75%, 80%, 85%, 90%, 95%, 96%, 97%, 98%, or 99% identical to the amino acid sequence of SEQ ID NO: 2. In some embodiments, a fatty acid transporter in accordance with the invention is capable of preferentially transporting monounsaturated fatty acids with C16 and C18 carbon chain lengths (e.g., palmitoleic acid and/or oleic acid) over a lipid bilayer membrane and has an amino acid sequence that is at least 75%, 80%, 85%, 90%, 95%, 96%, 97%, 98%, or 99% identical to the amino acid sequence of SEQ ID NO: 3.

In some embodiments, the amino acid sequence of a fatty acid transporter in accordance with the invention is the amino acid sequence of SEQ ID NO: 1. In some embodiments, the amino acid sequence of a fatty acid transporter in accordance with the invention is the amino acid sequence of SEQ ID NO: 2. In some embodiments, the amino acid sequence of a fatty acid transporter in accordance with the invention is the amino acid sequence of SEQ ID NO: 3.

### Nucleic Acids Encoding Fatty Acid Transporters

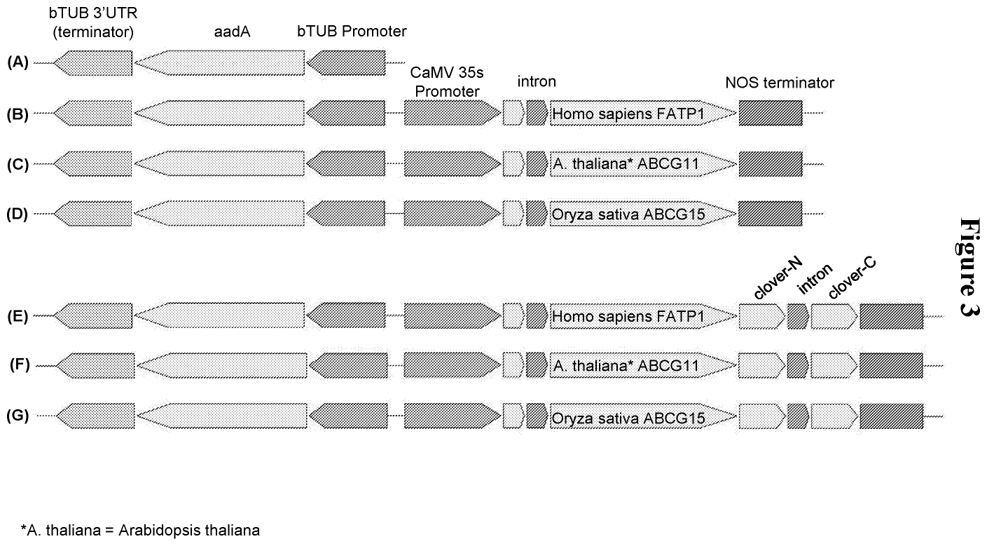

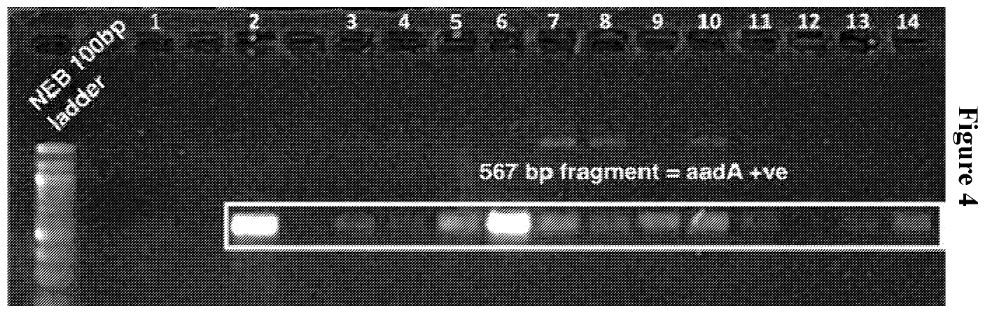

Nucleic acids comprising a coding sequence for a fatty acid transporter of the invention can be used to engineer single-celled photosynthetic organisms that are capable of transporting fatty acids across the cytoplasmic membrane from inside the cell. Exemplary nucleic acids are schematically illustrated in FIG. 3.

A suitable nucleic acid comprises a coding sequence for a fatty acid transporter operationally linked to a promoter sequence. The coding sequence may encode the amino acid sequence of one of SEQ ID NOs: 1 to 3. Typically, the promoter sequence encodes a strong promoter, such as a viral promoter (e.g., a Cauliflower Mosaic Virus 35S promoter encoded by SEQ ID NO: 7 or 8). In some embodiments, a suitable promoter native to the target organisms may be used. More typically, a suitable promoter is exogenous to the target organism.

Typically, the nucleic acid also comprises a terminator sequence (e.g., one of SEQ ID NOs: 22 or 23) operationally linked to the coding sequence. The presence of a terminator sequence prevents that transcription of sequences that are located downstream of the insertion site of the exogenous nucleic acid in the engineered organism is controlled by the promoter sequence which controls expression of the fatty acid transporter coding sequence. A suitable terminator sequence is, e.g., a nopaline synthase (NOS) terminator as encoded by SEQ ID NO: 22.

In some embodiments, the nucleic acid also includes a selection marker sequence. The selection marker can be used to select organisms that include the exogenous nucleic sequence in their genome. A suitable selection marker sequence for use with the invention encodes aminoglycoside-3′-adenyltransferase. The selection marker is operationally linked to a suitable promoter sequence. Suitable promoters include constitutively active promoters (e.g., promoters that control expression of tubulin).

In some embodiments, additional elements are included, e.g., to facilitate the detection of the fatty acid transporter upon expression inside the cell (for instance, to ensure its correct localization). For example, the coding sequence for the fatty acid transporter may be fused to a coding sequence encoding a marker polypeptide (e.g., a fluorescent marker protein such as a clover protein). The resulting fusion protein can be easily be detected by fluorescence microscopy.

### Coding Sequence

A coding sequence for a fatty acid transporter suitable for use with the invention may be codon-optimized for expression in the target organisms, e.g., a suitable single-celled photosynthetic organism. Codon optimization may be performed with any suitable algorithm known to the skilled person.

Moreover, the presence of introns into the genes may aid expression following transformation of a eukaryotic cell. Accordingly, in some embodiments, the coding sequence comprises one or more introns. For example, the Intronserter tool may be used to generate a codon-optimized, intron-containing coding sequence for optimal expression in the single-celled photosynthetic organism (Jaeger et al. Intronserter, an advanced online tool for design of intron containing transgenes, Algal Research, Volume 42, 2019). Accordingly, in some embodiments, a nucleic acid for use with the invention comprises a codon-optimized, intron-containing coding sequence for a fatty acid transporter.

### Exemplary Optimized Coding Sequences Encoding a Fatty Acid Transporter

Exemplary coding sequences for a fatty acid transporter optimized for expression in a target organisms of the invention are provided in Table 2.

TABLE 2
Exemplary optimized coding
sequences encoding a fatty acid transporter

| | SEQ ID | |
|---|---|---|
| NAME | NO: | SEQUENCE |
| Codon- | 4 | ATGGAGATCGAGGCCAGCCGCCAGCAGACCACCGT |
| optimized | | GCCCGTGAGCGTGGGCGGCGGCAACTTCCCCGTGG |
| Arabidopsis | | GCGGCCTGAGCCCCCTGAGCGAGGCTATCTGGCGC |
| thaliana | | GAGAAGGCTCCCACCGAGTTCGTGGGCGATGTGAG |
| ABCG11 | | CGCTCGCCTGACCTGGCAGGATCTGACCGTGATGGT |
| coding | | GACCATGGGCGATGGTGAGACTCAGAACGTGCTGG |
| sequence | | AGGGTCTGACTGGCTACGCCGAGCCCGGCAGCCTG |
| | | ACCGCTCTGATGGGCCCTAGCGGCAGCGGTAAGAG |
| | | CACCATGCTGGATGCTCTGGCTAGCCGCCTGGCCGC |
| | | TAACGCTTTCCTGAGCGGTACCGTGCTGCTGAACGG |
| | | TCGCAAGACCAAGCTGAGCTTCGGTACCGCTGCCTA |
| | | CGTGACCCAGGACGATAACCTGATCGGCACCCTGA |
| | | CCGTGCGCGAGACTATCTGGTACAGCGCTCGCGTGC |
| | | GCCTGCCCGACAAGATGCTGCGCAGCGAGAAGCGC |
| | | GCCCTGGTGGAGCGCACCATCATTGAGATGGGCCT |
| | | GCAGGACTGCGCTGACACCGTGATTGGCAACTGGC |
| | | ACCTGCGCGGCATCAGCGGCGGCGAGAAGCGCCGC |
| | | GTGAGCATCGCTCTGGAGATCCTGATGCGCCCCCGC |
| | | CTGCTGTTTCTGGACGAGCCCACCAGCGGCCTGGAC |
| | | AGCGCCAGCGCTTTCTTTGTGACCCAGACCCTGCGC |
| | | GCTCTGAGCCGCGATGGCCGCACTGTGATCGCTAGC |
| | | ATTCACCAGCCCAGCTCTGAGGTGTTTGAGCTGTTT |
| | | GACCGCCTGTACCTGCTGAGCGGTGGCAAGACCGT |
| | | GTACTTCGGCCAGGCCAGCGACGCTTACGAGTTCTT |
| | | CGCCCAGGCCGGCTTCCCTTGCCCCGCTCTGCGCAA |
| | | CCCTAGCGACCACTTCCTGCGCTGCATCAACAGCGA |
| | | TTTCGACAAGGTGCGCGCCACCCTGAAGGGCAGCA |
| | | TGAAGCTGCGCTTCGAGGCTAGCGACGATCCCCTG |
| | | GAGAAGATCACCACCGCCGAGGCCATCCGCCTGCT |
| | | GGTGGACTACTACCACACCAGCGACTACTACTACA |
| | | CCGCCAAGGCTAAGGTGGAGGAGATCAGCCAGTTC |
| | | AAGGGCACCATCCTGGACAGCGGCGGCAGCCAGGC |
| | | CAGCTTCCTGCTGCAGACCTACACCCTGACCAAGCG |
| | | CAGCTTCATCAACATGAGCCGCGACTTCGGCTACTA |
| | | CTGGCTGCGCCTGCTGATCTACATCCTGGTGACCGT |
| | | GTGCATCGGCACCATCTACCTGAACGTGGGCACCA |
| | | GCTACAGCGCTATCCTGGCCCGCGGCAGCTGCGCC |
| | | AGCTTCGTGTTCGGCTTCGTGACCTTCATGAGCATC |
| | | GGCGGCTTCCCCAGCTTCGTGGAGGACATGAAGGT |
| | | GTTCCAGCGCGAGCGCCTGAACGGCCACTACGGCG |
| | | TGGCTGCCTTCGTGATCGCCAACACCCTGAGCGCTA |
| | | CCCCCTTCCTGATCATGATCACCTTCATCAGCGGCA |
| | | CCATCTGCTACTTCATGGTGGGCCTGCACCCCGGCT |
| | | TCACCCACTACCTGTTCTTCGTGCTGTGCCTGTACG |
| | | CCAGCGTGACCGTGGTGGAGAGCCTGATGATGGCT |
| | | ATCGCCAGCATCGTGCCCAACTTCCTGATGGGCATC |
| | | ATCATCGGCGCCGGCATCCAGGGCATCTTCATGCTG |
| | | GTGAGCGGCTTCTTCCGCCTGCCCAACGACATCCCC |
| | | AAGCCCTTCTGGCGCTACCCCATGAGCTACATCAGC |
| | | TTCCACTTCTGGGCCCTGCAGGGCCAGTACCAGAAC |
| | | GACCTGCGCGGCCTGACCTTCGACAGCCAGGGCAG |
| | | CGCTTTCAAGATCCCCGGCGAGTACGTGCTGGAGA |
| | | ACGTGTTCCAGATCGACCTGCACCGCAGCAAGTGG |
| | | ATCAACCTGAGCGTGATCCTGAGCATGATCATCATT |
| | | TACCGCATCATTTTCTTTATCATGATTAAGACCAAC |
| | | GAGGACGTGACCCCTTGGGTGCGCGGCTACATCGC |
| | | TCGCCGCCGCATGAAGCAGAAGAACGGCACCCAGA |
| | | ACACTACTGTGGCTCCTGACGGCCTGACCCAGAGCC |
| | | CTAGCCTGCGCAACTACATTGCTACCCGCACTGACG |
| | | GTGCTCGCCGCTGG |
| Codon- | 5 | ATGTGCCTGCTGGAGGAGATGATGGAGATCAGCAG |
| optimized | | CAACGAGGAGATGATGGAGATGGCCATCGTGGAGC |
| Oryza sativa | | AGCTGCCCCCCAGCAGCCACCACCTGAACGGCGGT |
| ABCG15 | | AGCGTGGAGGTGGACATGGAGGAGGATCACGTGTG |
| coding | | GCCCACCAAGGACGGCCCTCTGCCCATTTTCCTGAA |
| sequence | | GTTCGAGAACGTGGAGTACAAGGTGAAGCTGACCC |
| | | CTAAGAACCCCCTGACTGCCGCTCGCGTGGCCTTCG |
| | | CTAGCCACAAGAGCACCGAGGACCAGGGCAGCTGC |
| | | AAGCACATCCTGAAGGGCGTGGGCGGTAGCGTGGA |
| | | TCCTGGCGAGATCCTGGCCCTGATGGGCCCCAGCG |
| | | GCAGCGGTAAGACCACTCTGCTGAAGATCCTGGGC |
| | | GGTCGCCTGAGCGGCGGTGTGCGCGGCCAGATCAC |
| | | CTACAACGACACCCCTTACAGCCCCTGCCTGAAGCG |
| | | CCGCATTGGCTTCGTGACTCAGGATGACGTGCTGTT |
| | | CCCTCAGCTGACCGTGGAGGAAACCCTGGTGTTCGC |
| | | CGCTTTCCTGCGCCTGCCCGCCCGCATGAGCAAGCA |
| | | GCAGAAGCGCGACCGCGTGGATGCTATCATTACTG |
| | | AGCTGAACCTGGAGCGCTGCCGCCACACCAAGATC |
| | | GGCGGTGCCTTCGTGCGCGGCGTGAGCGGCGGTGA |
| | | GCGCAAGCGCACCAGCATTGGCTACGAGATCCTGG |
| | | TGGATCCTAGCCTGCTGCTGCTGGACGAGCCCACTA |
| | | GCGGTCTGGACAGCACCAGCGCCGCTAAGCTGCTG |
| | | GTGGTGCTGCGCCGCCTGGCCCGCAGCGCCGCTCGC |
| | | CGCACCGTGATCACCACTATTCACCAGCCTAGCAGC |
| | | CGCATGTTCCACATGTTCGACAAGCTGCTGCTGGTG |
| | | GCCGAGGGCCACGCCATCTACCACGGCGGTGCCCG |
| | | CGGCTGCATGCGCCACTTCGCCGCTCTGGGCTTCAG |
| | | CCCCGGTATTGCCATGAACCCTGCCGAGTTCCTGCT |
| | | GGACCTGGCCACCGGCAACCTGGACGGTATCAGCA |
| | | GCCCCGCTAGCCTGCTGCTTCCTAGCGCCGCCGCTG |
| | | CTAGCCCCGATTCTCCTGAGTTCCGCAGCCACGTGA |
| | | TTAAGTACCTGCAGGCTCGTCACCGCGCTGCTGGCG |
| | | AGGAGGAAGCCGCTGCCGCCGCCGCTCGTGAGGGT |
| | | GGTGGTGGTGGTGGTGCCGGCCGCGACGAGGCCGC |
| | | TAAGCAGCTGCGCATGGCCGTGCGCATGCGCAAGG |
| | | ATCGCCGCGGCGGTATTGGCTGGCTGGAGCAGTTC |
| | | ACCGTGCTGAGCCGCCGCACTTTCCGCGAGCGCGCC |
| | | GCTGACTACCTGGATAAGATGCGCCTGGCCCAGAG |
| | | CGTGGGCGTGGCCCTGCTGCTGGGCCTGCTGTGGTG |
| | | GAAGAGCCAGACCAGCAACGAGGCTCAGCTGCGCG |
| | | ACCAGGTGGGTCTGATCTTCTACATTTGCATCTTCT |
| | | GGACCAGCAGCAGCCTGTTCGGCAGCGTGTACGTG |
| | | TTCCCTTTCGAGAAGCTGTACCTGGTGAAGGAGCGC |
| | | AAGGCCGACATGTACCGCCTGAGCGCCTACTACGC |
| | | TAGCAGCACTGTGTGCGATGCCGTGCCCCACGTGGT |
| | | GTACCCTGTGCTGTTCACCGCCATTCTGTACTTCAT |
| | | GGCTGACCTGCGCCGCACCGTGCCCTGCTTCTGCCT |
| | | GACTCTGCTGGCCACCCTGCTGATCGTGCTGACCAG |
| | | CCAGGGCACTGGTGAGCTGCTGGGCGCCGCTATTCT |
| | | GAGCGTGAAGCGCGCCGGCGTGATGGCTAGCCTGG |
| | | TGCTGATGCTGTTCCTGCTGACCGGCGGTTACTACG |
| | | TGCAGCACATCCCCAAGTTCATTCGCTGGCTGAAGT |
| | | ACGTGAGCTTCATGCACTACGGCTTCAACCTGCTGC |
| | | TGAAGGCCCAGTACCACGGCCACCTGACCTACAAC |
| | | TGCGGCAGCCGCGGCGGTTGCCAGCGCCTGCAGAG |
| | | CAGCCCTAGCTTCGGTACCGTGGACCTGGATGGCG |
| | | GTATGCGCGAGGTGTGGATCCTGCTGGCTATGGCCG |
| | | TGGCCTACCGCCTGCTGGCTTACCTGTGCCTGCGCA |
| | | AGCGCATTAGCCTGATGCCCCTA |
| Codon- | 6 | ATGCGCGCCCCCGGCGCCGGCGCCGCCAGCGTGGT |
| optimized | | GAGCCTGGCCCTGCTGTGGCTGCTGGGCCTGCCCTG |
| Homo sapiens | | GACCTGGAGCGCCGCCGCCGCCCTGGGCGTGTACG |
| FATP1 coding | | TGGGTAGCGGCGGTTGGCGCTTCCTGCGCATCGTGT |
| sequence | | GCAAGACCGCCCGCCGTGACCTGTTTGGCCTGAGC |
| | | GTGCTGATCCGCGTGCGCCTGGAGCTGCGCCGCCAC |
| | | CAGCGTGCTGGCCACACCATCCCCCGCATCTTCCAA |
| | | GCCGTGGTGCAGCGCCAGCCCGAGCGCCTGGCCCT |
| | | GGTGGACGCTGGCACCGGTGAGTGCTGGACCTTCG |
| | | CTCAGCTGGACGCCTACAGCAACGCCGTGGCCAAC |
| | | CTGTTCCGTCAGCTGGGCTTCGCCCCCGGTGACGTG |
| | | GTGGCTATCTTCCTGGAGGGTCGCCCCGAGTTTGTG |
| | | GGCCTGTGGCTGGGTCTGGCTAAGGCCGGCATGGA |
| | | GGCCGCTCTGCTGAACGTGAACCTGCGCCGTGAGC |
| | | CCCTGGCCTTCTGCCTGGGCACCAGCGGTGCTAAGG |
| | | CCCTGATCTTCGGCGGTGAGATGGTGGCCGCTGTGG |
| | | CCGAGGTGAGCGGCCACCTGGGTAAGAGCCTGATC |
| | | AAGTTCTGCAGCGGCGACCTGGGTCCTGAGGGCAT |
| | | TCTGCCCGACACCCACCTGCTTGACCCCCTGCTTAA |
| | | GGAGGCCAGCACCGCTCCCCTGGCCCAGATCCCCA |
| | | GCAAGGGTATGGACGATCGCCTGTTCTACATCTACA |
| | | CCAGCGGCACCACCGGTCTGCCCAAGGCCGCTATT |
| | | GTGGTGCACAGCCGCTACTATCGCATGGCCGCTTTC |
| | | GGCCACCACGCCTACCGCATGCAGGCCGCTGACGT |
| | | GCTGTACGATTGCCTGCCTCTGTACCACAGCGCCGG |
| | | CAACATCATTGGCGTGGGTCAGTGCCTGATCTACGG |
| | | CCTGACCGTGGTGCTGCGTAAGAAGTTCAGCGCTA |
| | | GCCGCTTTTGGGACGATTGCATCAAGTACAACTGCA |
| | | CCGTGGTGCAGTACATTGGCGAGATTTGCCGCTACC |
| | | TGCTGAAGCAGCCCGTGCGCGAGGCCGAGCGCCGC |
| | | CACCGTGTGCGCCTGGCCGTGGGCAACGGCCTGCG |
| | | CCCCGCCATTTGGGAGGAGTTCACCGAGCGCTTCGG |
| | | CGTGCGCCAGATCGGTGAGTTCTACGGCGCCACCG |
| | | AGTGCAACTGCAGCATTGCCAACATGGACGGTAAG |
| | | GTGGGCAGCTGCGGTTTCAACAGCCGCATCCTGCCC |
| | | CACGTGTACCCTATCCGTCTTGTGAAGGTGAACGAG |
| | | GATACCATGGAGCTGCTGCGCGACGCCCAGGGTCT |
| | | GTGCATCCCTTGCCAGGCCGGCGAGCCCGGTCTGCT |
| | | TGTGGGTCAGATCAACCAGCAAGACCCCCTGCGCC |
| | | GCTTCGATGGTTACGTGAGCGAGAGCGCCACCAGC |
| | | AAGAAAATTGCCCACAGCGTGTTCAGCAAGGGTGA |
| | | CAGCGCCTACCTGAGCGGCGATGTGCTGGTGATGG |
| | | ATGAGCTGGGCTACATGTACTTCCGCGACCGCAGC |
| | | GGTGACACCTTTCGCTGGCGCGGTGAGAACGTGAG |
| | | CACCACTGAGGTGGAGGGCGTGCTGAGCCGCCTGC |
| | | TGGGCCAGACCGACGTGGCTGTGTACGGCGTGGCC |
| | | GTGCCCGGTGTGGAGGGCAAGGCCGGCATGGCCGC |
| | | TGTGGCTGACCCCCACAGCCTGCTGGATCCCAACGC |
| | | TATCTACCAGGAGCTGCAGAAGGTGCTGGCCCCCT |
| | | ACGCTCGCCCTATTTTCCTGCGCCTGCTGCCCCAGG |
| | | TGGACACCACCGGTACCTTCAAGATCCAAAAAACC |
| | | CGCCTGCAGCGCGAGGGCTTCGACCCCCGCCAGAC |
| | | CAGCGATCGCCTGTTCTTTCTGGATCTGAAGCAGGG |
| | | CCACTACCTGCCCCTGAACGAGGCTGTGTACACCCG |
| | | TATTTGCAGCGGCGCCTTCGCTCTG |

In some embodiments, an optimized coding sequence for a fatty acid transporter in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 4 and encodes the amino acid sequence of SEQ ID NO: 1. In some embodiments, an optimized coding sequence for a fatty acid transporter in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 5 and encodes the amino acid sequence of SEQ ID NO: 2. In some embodiments, an optimized coding sequence for a fatty acid transporter in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 6 and encodes the amino acid sequence of SEQ ID NO: 3.

### Promoter Sequences

A nucleic acid for use with the invention includes a promoter sequence for expression of the fatty acid transporter coding sequence following transformation into a target organism. The promoter may be a native (endogenous) promoter of the target organism. The coding sequence may be inserted into the genome of the organism to be operationally linked to a native promoter. Suitable endogenous promoter sequences may include promoter sequences that control the expression of constitutively active genes in the target organisms. More typically, the promoter sequence is exogenous to the target organism. Suitable exogenous promoter sequences may include promoter sequences of viral origin (e.g., a promoter sequence originating from a plant virus or viruses known to infect the target organism). Exemplary promoters suitable for use with the invention include Cauliflower Mosaic Virus (CaMV) 35s promoters (including enhanced versions).

Accordingly, in some embodiments, the promoter sequence may be derived from the genome of a different organism to the genome from which the coding sequence is derived. For example, the promoter sequence may be derived from the genome of the same organism to the genome from which the coding sequence is derived. More typically, the coding sequence and the promoter sequence are derived from different genomes.

In some embodiments, the promoter may be constitutively active. For example, the promoter sequence may be derived from a gene that is known to be constitutively active in the target organisms. Examples include promoter sequences from genes encoding, e.g., a photosystem I reaction center subunit II (PSAD), a ribulose bisphosphate carboxylase small subunit (RBCS), a tubulin (e.g., α-tubulin or β-tubulin) and a ubiquitin extension protein. Exemplary constitutively active promoters suitable for use with the invention include the Nannochloropsis gaditana β-tubulin2 promoter, the Nannochloropsis gaditana ubiquitin extension protein promoter, the C. reinhardtii HSP70A/RBCS2 hybrid promoter, the C. reinhardtii photosystem I reaction center subunit II (PSAD) promoter, and the C. reinhardtii ribulose bisphosphate carboxylase small subunit (RBCS) promoter.

In some embodiments, the promoter sequence may be a non-naturally occurring hybrid promoter sequence of two different promoter sequences. An exemplary hybrid promoter is the C. reinhardtii HSP70A-RBCS2 promoter.

In other embodiments, the promoter sequence may be an inducible promoter. An inducible promoter may induce expression of the coding sequence only under specific conditions (e.g., under certain growth or environmental conditions, including particular chemical or physical conditions). The inducible promoter may allow growth of the target organism without expression of the coding sequence, and may be used to induce expression of the coding sequence when the organism is cultured under specific growth conditions, e.g., under nitrogen depletion, vitamin depletion, or especially high-light environments. Exemplary inducible promoters suitable for use with the invention is Nannochloropsis gaditana and Phaeodactylum tricornutum Nitrogen Reductase (NR) promoters. Other suitable inducible promoter sequences are derived from the genes encoding methionine synthase (METE) and Vesicle-Inducing Protein in Plastids 2 (VIPP2), respectively.

### Exemplary Promoter Sequences

Exemplary promoter sequences for use with the invention are provided in Table 3.

TABLE 3
Exemplary promoter sequences

| | SEQ ID | |
|---|---|---|
| NAME | NO: | SEQUENCE |
| CaMV 35s | 7 | ACTCTCGTCTACTCCAAGAATATCAAAGATACAGTC |
| promoter | | TCAGAAGACCAAAGGGCTATTGAGACTTTTCAACA |
| (enhanced) | | AAGGGTAATATCGGGAAACCTCCTCGGATTCCATTG |
| | | CCCAGCTATCTGTCACTTCATCAAAAGGACAGTAGA |
| | | AAAGGAAGGTGGCACCTACAAATGCCATCATTGCG |
| | | ATAAAGGAAAGGCTATCGTTCAAGATGCCTCTGCC |
| | | GACAGTGGTCCCAAAGATGGACCCCCACCCACGAG |
| | | GAGCATCGTGGAAAAAGAAGACGTTCCAACCACGT |
| | | CTTCAAAGCAAGTGGATTGATGTGATAACATGGTG |
| | | GAGCACGACACTCTCGTCTACTCCAAGAATATCAA |
| | | AGATACAGTCTCAGAAGACCAAAGGGCTATTGAGA |
| | | CTTTTCAACAAAGGGTAATATCGGGAAACCTCCTCG |
| | | GATTCCATTGCCCAGCTATCTGTCACTTCATCAAAA |
| | | GGACAGTAGAAAAGGAAGGTGGCACCTACAAATGC |
| | | CATCATTGCGATAAAGGAAAGGCTATCGTTCAAGA |
| | | TGCCTCTGCCGACAGTGGTCCCAAAGATGGACCCCC |
| | | ACCCACGAGGAGCATCGTGGAAAAAGAAGACGTTC |
| | | CAACCACGTCTTCAAAGCAAGTGGATTGATGTGAT |
| | | ATCTCCACTGACGTAAGGGATGACGCACAATCCCA |
| | | CTATCCTTCGCAAGACCTTCCTCTATATAAGGAAGT |
| | | TCATTTCATTTGGAGAGGACACGCTGA |
| CaMV 35s | 8 | TGAGACTTTTCAACAAAGGGTAATATCGGGAAACC |
| promoter | | TCCTCGGATTCCATTGCCCAGCTATCTGTCACTTCAT |
| | | CAAAAGGACAGTAGAAAAGGAAGGTGGCACCTAC |
| | | AAATGCCATCATTGCGATAAAGGAAAGGCTATCGT |
| | | TCAAGATGCCTCTGCCGACAGTGGTCCCAAAGATG |
| | | GACCCCCACCCACGAGGAGCATCGTGGAAAAAGAA |
| | | GACGTTCCAACCACGTCTTCAAAGCAAGTGGATTG |
| | | ATGTGATATCTCCACTGACGTAAGGGATGACGCAC |
| | | AATCCCACTATCCTTCGCAAGACCTTCCTCTATATA |
| | | AGGAAGTTCATTTCATTTGGAGAGGACACGCTGA |
| C. reinhardtii | 9 | CTGGCACTTTCTTGCGCTATGACACTTCCAGCAAAA |
| bTUB2 promoter | | GGTAGGGCGGGCTGCGAGACGGCTTCCCGGCGCTG |
| | | CATGCAACACCGATGATGCTTCGACCCCCCGAAGCT |
| | | CCTTCGGGGCTGCATGGGCGCTCCGATGCCGCTCCA |
| | | GGGCGAGCGCTGTTTAAATAGCCAGGCCCCCGATT |
| | | GCAAAGACATTATAGCGAGC |
| C. reinhardtii | 10 | CTGAGGCTTGACATGATTGGTGCGTATGTTTGTATG |
| HSP70A-RBCS2 | | AAGCTACAGGACTGATTTGGCGGGCTATGAGGGCG |
| promoter | | GGGGAAGCTCTGGAAGGGCCGCGATGGGGCGCGCG |
| | | GCGTCCAGAAGGCGCCATACGGCCCGCTGGCGGCA |
| | | CCCATCCGGTATAAAAGCCCGCGACCCCGAACGGT |
| | | GACCTCCACTTTCAGCGACAAACGAGCACTTATACA |
| | | TACGCGACTATTCTGCCGCTATACATAACCACTCAG |
| | | CTAGCTTAAGATCCCATCAAGCTTGCATGCCGGGCG |
| | | CGCCAGAAGGAGCGCAGCCAAACCAGGATGATGTT |
| | | TGATGGGGTATTTGAGCACTTGCAACCCTTATCCGG |
| | | AAGCCCCCTGGCCCACAAAGGCTAGGCGCCAATGC |
| | | AAGCAGTTCGCATGCAGCCCCTGGAGCGGTGCCCT |
| | | CCTGATAAACCGGCCAGGGGGCCTATGTTCTTTACT |
| | | TTTTTACAATACTTGA |
| C. reinhardtii long | 11 | CGCGACGGTTCGAGAACCGACTTGAGGGCGCCAAA |
| HSP70A-RBCS2 | | CGAGCCCGAGCCGCCGTTGCGCCAGGCGAAACCAG |
| hybrid promoter | | AACCGTAGATTAATGCACTTGAGCTATTCATTGGAG |
| | | CGATCTGCCGGGGACAGCGGGTCTGGCGTGCGCGC |
| | | GATTGGAGATCGCAAATTACATATGTCTGCGTGACG |
| | | GCGGGGAGCTCGCTGAGGCTTGACATGATTGGTGC |
| | | GTATGTTTGTATGAAGCTACAGGACTGATTTGGCGG |
| | | GCTATGAGGGCGGGGGAAGCTCTGGAAGGGCCGCG |
| | | ATGGGGCGCGCGGCGTCCAGAAGGCGCCATACGGC |
| | | CCGCTGGCGGCACCCATCCGGTATAAAAGCCCGCG |
| | | ACCCCGAACGGTGACCTCCACTTTCAGCGACAAAC |
| | | GAGCACTTATACATACGCGACTATTCTGCCGCTATA |
| | | CATAACCACTCAGCTAGCGATCCCGGGCGCGCCAG |
| | | AAGGAGCGCAGCCAAACCAGGATGATGTTTGATGG |
| | | GGTATTTGAGCACTTGCAACCCTTATCCGGAAGCCC |
| | | CCTGGCCCACAAAGGCTAGGCGCCAATGCAAGCAG |
| | | TTCGCATGCAGCCCCTGGAGCGGTGCCCTCCTGATA |
| | | AACCGGCCAGGGGGCCTATGTTCTTTACTTTTTTAC |
| | | AAG |
| C. reinhardtii | 12 | GTAGGTCAGGACCAGAGCCTACAACATTGTAAGGC |
| METE promoter | | CATGTATCCTCCAGTGCCATTTGCGACGTTACACGA |
| | | CGAGACAATCTGCGCTGTGTTCAAGCACACACATCC |
| | | GGGACTCGTGTGATGTCTTAACATCTGCAAGGAAA |
| | | CATAAGGTGGCCGGCGTCACGCAAGATGACGCGAC |
| | | GGACTCGAATGCTTGCTTCGTCCAGCGACAAATAA |
| | | GGACAGGCAGGGCGCCTGCATTGCGTGCGCTTAGA |
| | | GGCGAGGCGCTCAAGACATTTCGGCAGCAATAATT |
| | | GGTATTGAGGCACATTCTGCACCAGGATGCCAAGA |
| | | GGTGAACGTTGCTGCCTTAATGTATATCTGCACAGC |
| | | TGGCCGGTTACTGCGAACCGGGTGCCTTTTGGAGTC |
| | | GCTCCTAAAAGACGTCACCGGCGCAACGTCGTCGT |
| | | GCGGCCGTACAGGTATGCAAAATTGGCCCGGTTGC |
| | | GCGCAGGCCGATTATTTAAGTGACAT |
| C. reinhardtii | 13 | CCCGCCAGCCCCCGCTCCTCTGCTGCCTCTGATGCC |
| NIT1 promoter | | TCATGCCAAAAGTCCTGACGCGGCGCCCTCACATCC |
| | | CCGTCCGGGTAATCTATGAGTTTCCCTTATCGAGCA |
| | | TGTACGCGATAGTGGACGGGGCTCAGGGTGGGGGG |
| | | TGGGTGGGTGGGAGGGGCGTTCCTTCAGACACCCT |
| | | GGAGGGGTGGCTAGAAAAGCGGCCGCGCGCCAGA |
| | | AATGTCTCGCTGCCCTGTGCAATAAGCACCGGCTAT |
| | | ATT |
| C. reinhardtii | 14 | GATCCCACACACCTGCCCGTCTGCCTGACAGGAAGT |
| PSAD promoter | | GAACGCATGTCGAGGGAGGCCTCACCAATCGTCAC |
| | | ACGAGCCCTCGTCAGAAACACGTCTCCGCCACGCTC |
| | | TCCCTCTCACGGCCGACCCCGCAGCCCTTTTGCCCT |
| | | TTCCTAGGCCACCGACAGGACCCAGGCGCTCTCAG |
| | | CATGCCTCAACAACCCGTACTCGTGCCAGCGGTGCC |
| | | CTTGTGCTGGTGATCGCTTGGAAGCGCATGCGAACA |
| | | CGAAGGGGCGGAGCAGGCGGCCTGGCTGTTCGAAG |
| | | GGCTCGCCGCCAGTTCGGGTGCCTTTCTCCACGCGC |
| | | GCCTCCACACCTACCGATGCGTGAAGGCAGGCAAA |
| | | TGCTCATGTTTGCCCGAACTCGGAGTCCTTAAAAAG |
| | | CCGCTTCTTGTCGTCGTTCCGAGACATGTTAGCAGA |
| | | TCGCAGTGCCACCTTTCCTGACGCGCTCGGCCCCAT |
| | | ATTCGGACGCAATTGTCATTTGTAGCACAATTGGAG |
| | | CAAATCTGGCGAGGCAGTAGGCTTTTAAGTTGCAA |
| | | GGCGAGAGAGCAAAGTGGGACGCGGCGTGATTATT |
| | | GGTATTTACGCGACGGCCCGGCGCGTTAGCGGCCCT |
| | | TCCCCCAGGCCAGGGACGATTATGTATCAATATTGT |
| | | TGCGTTCGGGCACTCGTGCGAGGGCTCCTGCGGGCT |
| | | GGGGAGGGGGATCTGGGAATTGGAGGTACGACCGA |
| | | GATGGCTTGCTCGGGGGGAGGTTTCCTCGCCGAGC |
| | | AAGCCAGGGTTAGGTGTTGCGCTCTTGA |
| C. reinhardtii | 15 | AAACAGCACAGAAGGGAAAGGCCGGGTGTCGGAA |
| VIPP2 promoter | | AAACGCGCTCCGGAACGGCCTTCCTGAAGCTCGCG |
| | | CACATTCCCACCCTTTCACCCCGCAATAACCGCTCG |
| | | CCGCTCGTTGCTAATGCTGATAGTTGTTGCGCACTT |
| | | GCGCCCACGCGCCGGTGGGACAGCCCCGGCAAGCC |
| | | AGATTTCTCACCGGCGTCACCATCCGCAGCCGCAGC |
| | | GGCAGCGCCTGCCACCAGCAGCGCCGCAAGTGCCA |
| | | GCGCCGGCAGAAGCAGTCCCGCGCATTGGTGGGTT |
| | | GGCATGGTTGTTTTGGCGGGTGCGCGCAAAGAGCT |
| | | CTGGTAGACTACGGCGAGTCAAGCGGCGCGGTCGC |
| | | ACGGAGAAAGGTCGGGCCCTGGAAAAGCGTTTCGG |
| | | AAGGGGGCGTCGGTGCAGCTAGGACGGGGAATGAC |
| | | GAGAGGTGGCTGCTCGCGGGGGAGCACAGCTGCAG |
| | | GCCGGCCGCGGACTTGCGCGGAGCTGCGCCGGGAG |
| | | GCCGAGGCCGACCCCCCGCGCCCCATGCAAAAGGC |
| | | GAAGGCCCCTGCGTAAATTCGCGCACGCCAAACTT |
| | | ATAGGGTTTGAACTATAAGCA |
| Chlorella vulgaris | 16 | TGATGCAAACAATTCTATTGATCATCATGCTGCATA |
| α-tubulin | | CTTCAATGCCTTGATGCACAAGCTCTCACAACAAGC |
| promoter | | ATGTCCTCCCGCATGCACATCGTTTCCCCCTCACAA |
| | | AATCTGTCTCTGGTTTGGTGGTCAGCTGGCGTGTTT |
| | | TGCGGGCGTTCCCTCTTTCAGACACCACACCAGAGC |
| | | GTCGCGCGCAAAACTTACAAAATTGGATGACGCGT |
| | | CCTCGTTCGCCGTCTGCTCCTCTTTCTTAAAGCCTCC |
| | | AAGCCACCCAGCTTCTTTCTAAATCTGCAACCATG |
| Dunaliella | 17 | CTTTTGTCTGTACAGACCTGGTTTGCTCAAGCCTAG |
| tertiolecta RBCS | | CAGCGCATTGGAAATCCACTGGAAATCCCAAATGT |
| promoter | | GGCGCACAGGAATATGCTCAGGACTTTTTCTTGCTC |
| | | GTGCGCACCTGATGTCAACCCTTCTCAAGGTTTGGC |
| | | GCACCTAGGAAGCACCCTCACTGAGGTGCTTCCTTG |
| | | AGAGCCCATGCAATCAAAACACCTGGTCCACTGAC |
| | | ACCCATTTACTGTATCAAGGCGATTGCTTGATAAAT |
| | | GAGGCTGTGCAGGATTGGAGGCGGTGCGTTCATCT |
| | | GCTGCTTGCAGGCTCTTCCCAGCACTTGCATTCTTC |
| | | CTTGCATCCTCCACTCAGCTCACAAGCCCACCCCTG |
| | | AAAGCACTCACGCAGCACAACCCGCTGCATTTTAA |
| | | ACAACGCTGTCCAACAGGGCCTCCTCACTCTCACAC |
| | | TACCTCAACAAACAAGGGGAAGTTGGTTACTA |
| Nannochloropsis | 18 | ACTTGTGCGTGGTTACTTGCCGACAGAATGGCGTGT |
| gaditana β-tubulin | | CCTTATTCGGCCTGGTGACCGTCCCGGTCGTAGAAC |
| promoter | | GTTCTGTCTCCCACCCTTCCTTCCAAGGCCTGAAGT |
| | | CGTTTTGTGACCGTAATTTTCAATTTGCGTGCAAGG |
| | | CCTACAAGTGAGGGCTTTATCTATGCGCATTTGACA |
| | | TGGTGCTGCTTATCAACGCTTATCCTGTCCGGCCCC |
| | | ATTTCCACGCAACGTCATTTTTTGACAGACTAAC |
| Nannochloropsis | 19 | GGCTTCTCACGTCGTTCTCATCGTCTGCTTTCTCCCC |
| gaditana nitrogen | | CTTTCCTTTTCTTCCGTCGTTTCTCCCACAGAACAAT |
| reductase | | TACGGGAGATGCTGGATTTCTATGTGGAGGCTGAC |
| promoter | | GTTTTCCGGGTCAGCGCCGATCGGACGATGATTGAA |
| | | CTCTTCAACACGGATTGAGAGAGCACTGTACGCAG |
| | | TGCAGAGCGTGGTAGGACATAGAATCGCGCAGGGA |
| | | AATAGGTTGGAAGGGGAGCAATTCGCAGAAGGAAA |
| | | GCTCAGCGATCGAGAAAAACATGGAGGGTGAAGAT |
| | | AAGCTCGCTCTGAAAGTAAATCTGACAAATCAAGC |
| | | ACAGATGAGGTCACTGCGAAGTTGACCTGAAAGCG |
| | | TGTTTTTCAAATCGTCGCCCAGGCGAAGTGTTATTA |
| | | TTTCTTGCACATCTTGAAGATAGTCTATACGCAGGA |
| | | TGAAAAGGAGGATCAACTTTGCCATTTCTGCATACG |
| | | TCATAATTCCACGATGACTAGCAATGTGGAATAGAT |
| | | CTTCCTTTTGAATTTCTCTGAATGGTTAGCACTTTCC |
| | | TTTGTTTTTTTGTCCTATGAAATTTAAATGGTAATTG |
| | | ATACCTCCACTAAGGAGTCAAATCAATAGCTTTGAT |
| | | CACAAGCAGAAGAAATGATAAGAGCGCAAAGCTTG |
| | | GAATAATCTCTTTATATGTCCCATTGATTAACATCT |
| | | ATCCTACAAGGGCGTGAACAACCACGATCAGAGTT |
| | | TAGGAGAGCGATCAACTCCAAGCACTAAAAGGGCG |
| | | TGAACAACCACTATAAGAATTGAAGAGGCGATCCT |
| | | CCAAGAACGCTGCCCGACCAGGCATTAATTGCCGC |
| | | ATAATCTGACCGTCGTCTGGATAGCCTGGCAGACG |
| | | GGAAAACTTCATGGAATAATGACGCCATTACTTGA |
| | | AGTTTCGCGGGGCTTTTTTTCCTGGCTCGGAATTAT |
| | | ACTTTTTACGCCATAACTGCGTTTCCCCGTTCTCCAA |
| | | GGGCTCCGCCGTCGGCAACTTTGTATGGGGAAGCG |
| | | CGGTAGATTTTGTTTTCAAGGAAGGACCAAGGGTGT |
| | | GAGGTGGCTTTTGAAACACTGGTGCCATGTATGGG |
| | | ACTATTCTGACGGATCTCCGCCGCGCAACTTAACTC |
| | | CCGCCTCGGATTCCGTGTTAGGCGTCGTAACGAACA |
| | | TTAGGAAGACATCGGATCTTAGATGAGAAATTATG |
| | | CATCATGCGACTTGTGTGAATGTGCATCAATCTTTT |
| | | GCCTGTTGCATTACGCATGCTCACGCGACTTCATGA |
| | | ATATGTCTTCCCTTACATACTCCTCCTGATCACGAC |
| | | AACACAACATTTCTCACATCACTCCAGGGTGGTCG |
| Nannochloropsis | 20 | CATCCTGCTGTATGATTTTGGCACAACGACGCGGTT |
| gaditana ubiquitin | | GGAGGGTGAGGGAGATGGTGGTGAGACATCAGAA |
| extension protein | | ATGGAGAGAATAACGGTAGAGAAAAAAAGGAACA |
| promoter | | CACGCGCCCAGGATAGAACCAGGACCACATTCCAA |
| | | GTCCCTCACCAGCAACAAGAGGGACACCACGTCGG |
| | | TGAAGAGGTGCGTGAGACAGAGGACACCAGACAAC |
| | | GTGCACAAAGCGTCGGAAAGGGAGGCGACCGCTTC |
| | | GCTGGAGGGGGGCCGTCCACAGCCTGCGCCCAACC |
| | | ACACACATTCCTCCCGCGAGGCTCAAGTGCCATGCC |
| | | GCCGAGTGCCATGCTCCTGCATCCGGGGCAGGTCG |
| | | ACGAGCCAGCATGGTTTGGTCGCCGACAACGCAGA |
| | | TTTGATGGAGCTTGGGCATTTCTTTACCGGACACGT |
| | | CGCTTCTTACATTTTTACATTCCAATTCAGAGGTCA |
| | | CACCCGCGATCGAGAAAGCAGAGAGTCGGACCCCC |
| | | TTCAAGCAGGAAAGGCAAGCAACATACCCATCGTT |
| | | AAGTCTGATGATGGCTGGTGCAAAGCCAAGGCAGC |
| | | CAGTCGATATGTTAATGATCGGGTACGTTTAAAGGC |
| | | GTGGGCAAGGTTATTTAACGCTGGTAGCATCGTAG |
| | | AGTTTTTAACGCAACTGAAGATTTCATGTCTGACTT |
| | | GGAGATACGGTCGGGGCAGCAGCT |
| Phaeodactylum | 21 | CATATGCGGAAGTGACTGTAAACGAGAAGTGCACG |
| tricornutum | | AAGCCTTTTCTTGTGACGTCACAAACCGAACAGCCC |
| nitrate reductase | | TACGTGGCGTGCGACTTTCCGGCACTTTGGATATCG |
| promoter | | TTGCCCTATATGTATTGGGTGTATACATGTCGTATT |
| | | CCAACTACGACAGAAAACAGTCAGTTACGGGTATT |
| | | CGAACTTCCGGCACCTGCAGCGAGGAGTTTTGTGTC |
| | | CGTCGCACATCCTCCGTGCTTTCGGCACCTACGTAG |
| | | GCGGCAAACTTCCTGCCTTCCGCCCGCCTTGCGGCA |
| | | TTCCGGCTCCGGGCCAGAATTGCCCGGGTGTTCACA |
| | | ATTTTGCCTCCTCACGAAAAAACGTTCTACTTTGTA |
| | | TTTTGGTCGGGTTTCGGATCCTTCCAGCAACCATTC |
| | | TCATTCAAAGTCACCACTTGTGCGAACG |

### Terminator Sequences

A nucleic acid for use with the invention may comprise a terminator sequence. The terminator sequence may be located at the 3′ end of the coding sequence for the fatty acid transporter. A suitable terminator sequence is capable of terminating transcription of the coding sequence in the target organism. In some embodiments, a suitable promoter sequence and a suitable terminator sequence are derived from the genomes of different organisms. In some embodiments, the promoter sequence, the coding sequence and the terminator sequence are derived from different organisms. The terminator sequence may be a native (endogenous) terminator of the target organism. More typically, the terminator sequence is exogenous to the target organism.

An exemplary terminator sequence suitable for use with the invention is the Agrobacterium tumefaciens nopaline synthase (NOS) terminator. Another exemplary terminator sequence suitable for use with the invention is the PSAD terminator native to Chlamydomonas. A further exemplary terminator suitable for use with the invention is the Phaeodactylum tricornutum nitrate reductase terminator.

### Exemplary Terminator Sequences

Exemplary terminator sequences for use with the invention are provided in Table 4.

TABLE 4
Exemplary terminator sequences

| | SEQ ID | |
|---|---|---|
| NAME | NO: | SEQUENCE |
| Agrobacterium | 22 | GATCGTTCAAACATTTGGCAAT |
| tumefaciens | | AAAGTTTCTTAAGATTGAATCC |
| NOS | | TGTTGCCGGTCTTGCGATGATT |
| terminator | | ATCATATAATTTCTGTTGAATT |
| | | ACGTTAAGCATGTAATAATTAA |
| | | CATGTAATGCATGACGTTATTT |
| | | ATGAGATGGGTTTTTATGATTA |
| | | GAGTCCCGCAATTATACATTTA |
| | | ATACGCGATAGAAAACAAAATA |
| | | TAGCGCGCAAACTAGGATAAAT |
| | | TATCGCGCGCGGTGTCATCTAT |
| | | GTTACTAGATC |
| Phaeodactylum | 23 | AACCTTCCTTAAAAATTTAATT |
| tricornutum | | TTCATTAGTTGCAGTCACTCCG |
| nitrate | | CTTTGGTTTCACAGTCAGGAAT |
| reductase | | AACACTAGCTCGTCTTCACCAT |
| terminator | | GGATGCCAATCTCGCCTATTCA |
| | | TGGTGTATAAAAGTTCAACATC |
| | | CAAAGCTAGAACTTTTGGAAAG |
| | | AGAAAGAATATCCGAATAGGGC |
| | | ACGGCGTGCCGTATTGTTGGAG |
| | | TGGACTAGCAGAAAGTGAGGAA |
| | | GGCACAGGATGAGTTTTCTCGA |
| | | G |

### Selection Marker

A nucleic acid for use with the invention may comprise a selection marker. The selection marker allows for selection of organisms in which transformation has been successful. In one embodiment, the selection marker provides antibiotic resistance. Suitable genes encoding a selection marker (e.g., an antibiotic resistance) include aadA (aminoglycoside-3′-adenyltransferase, confers resistance against spectinomycin), AphVII (confers resistance against hygromycin), APhVIII (confers resistance against paromomycin), NptII (confers resistance against neomycin phosphotransferase II, against kanamycin), Ble (from Streptoalloteichus hindustanus, confers resistance against zeocin or bleomycin), CAT (chloramphenicol acetyltransferase, confers resistance against chloramphenicol) and BSR (from Streptomyces Griseochromogenes, confers resistance against blasticidin). In some embodiments, the selection marker is aminoglycoside-3′-adenyltransferase. Aminoglycoside-3′-adenyltransferase provides resistance against spectinomycin. A nucleic acid for use with the invention may comprise a promoter sequence, a fatty acid transporter coding sequence and a terminator sequence (in 5′ to 3′ order) and include a selection marker sequence with a separate promoter and, optionally, a separate terminator sequences.

Accordingly, in some embodiments, a suitable selection marker is operationally linked to a promoter sequence and a terminator sequence. In some embodiments, the promoter and terminator sequences operationally-linked to the selection marker are different to the promoter and terminator sequences operationally-linked to the coding sequence.

### Additional Elements

A nucleic acid for use with the invention may comprise additional sequence elements. The nucleic acid may comprise a sequence coding for a marker polypeptide. Marker polypeptides include various tags, including His tags, c-myc tags. In some embodiments, the marker polypeptide is a protein, e.g., Glutathione-S-transferase (GST). In particular embodiments, the marker polypeptide is a fluorescent protein.

In a typical embodiment, the coding sequence of the marker polypeptide is fused to the coding sequence of the fatty acid transporter to encode a fusion protein. The coding sequence encoding the fusion protein may be operationally linked to a promoter sequence and a terminator sequence (located at the 5′ end and the 3′ end of the fusion protein coding sequence, respectively).

The inclusion of a marker polypeptide or protein can be used to detect expression of the fatty acid transporter protein following transformation into organisms. In some embodiments, the marker polypeptide or protein is used to localize the fatty acid transporter within the cell. For example, a fusion protein comprising the fatty acid transporter fused to a fluorescent marker protein may be used to detect and localize the fatty acid transporter by fluorescence microscopy.

In certain embodiments, the nucleic acid comprises a first coding sequence for a fatty acid transporter and a second coding sequence for fluorescent marker protein, whereby the first and second coding sequences are operationally linked for co-expression. Co-expression of the fatty acid transporter and the fluorescent marker protein may be used to identify an organism expressing the fatty acid transporter.

Suitable fluorescent marker sequences are known in the art. In some embodiments, the fluorescent marker is a clover fluorescent protein.

### Exemplary Fluorescent Marker Sequences

An exemplary fluorescent marker sequence for use with the invention is provided in Table 5.

TABLE 5
Exemplary fluorescent marker sequence

| | SEQ ID | |
|---|---|---|
| NAME | NO: | SEQUENCE |
| Clover | 24 | ATGATCGAGGGCAGGGTGAGCAAGGGCGAGGAGCT |
| fluorescent | | GTTCACCGGCGTGGTGCCCATCCTGGTGGAGCTGGA |
| protein coding | | CGGCGACGTGAACGGCCACAAGTTCAGCGTGCGCG |
| sequence | | GCGAGGGCGAGGGCGACGCCACCAACGGCAAGCTG |
| | | ACCCTGAAGTTCATCTGCACCACCGGCAAGCTGCCC |
| | | GTGCCCTGGCCCACCCTGGTGACCACCTTCGGCTAC |
| | | GGCGTGGCCTGCTTCAGCCGCTACCCCGACCACATG |
| | | AAGCAGCACGACTTCTTCAAGAGCGCCATGCCCGA |
| | | GGGCTACGTGCAGGAGCGCACCATCAGCTTCAAGG |
| | | ACGACGGCACCTACAAGACCCGCGCCGAGGTGAAG |
| | | TTCGAGGGCGACACCCTGGTGAACCGCATCGAGCT |
| | | GAAGGGCATCGACTTCAAGGAGGACGGTGAGCTTG |
| | | CGGGGTTGCGAGCAACACTCCAGCAACGAACAGTG |
| | | CCCAAGTCAGGAATCTGCAGTCAGCCTGGGCTTTCG |
| | | GCGGCTTTTTCTTGGGCAAACAGCTTGCACTCATGC |
| | | CAGCGCGGCTTGTCCAGCCTCACTTGAGCTTTCCAG |
| | | CTGCTACCAGCCGGGCTATACGACAGCGACAGAGC |
| | | CATAGCGTGGAATCACTTATTTGGGTTGCCGAAGTA |
| | | GCGGTCGGAGCGTGAGTTCTTGGTCAAGCCGCCCCT |
| | | TATCCGGTTCCTGTCCGTGTCTTTGTCCCTCGTTCAC |
| | | CCTTCGCGGCACCCTTCATCCCCTTGCTTGCAGGTA |
| | | ACATCCTGGGCCACAAGCTGGAGTACAACTTCAAC |
| | | AGCCACAACGTGTACATCACCGCCGACAAGCAGAA |
| | | GAACGGCATCAAGGCCAACTTCAAGATCCGCCACA |
| | | ACGTGGAGGACGGCAGCGTGCAGCTGGCCGACCAC |
| | | TACCAGCAGAACACCCCCATCGGCGACGGCCCCGT |
| | | GCTGCTGCCCGACAACCACTACCTGAGCCACCAGA |
| | | GCGCCCTGAGCAAGGACCCCAACGAGAAGCGCGAC |
| | | CACATGGTGCTGCTGGAGTTCGTGACCGCCGCCATC |
| | | GAGGGCAGGTGGAGCCACCCGCAGTTCGAGAAGTA |
| | | A |
| Clover | 25 | VSKGEELFTGVVPILVELDGDVNGHKFSVRGEGEGDA |
| fluorescent | | TNGKLTLKFICTTGKLPVPWPTLVTTFGYGVACFSRYP |
| protein | | DHMKQHDFFKSAMPEGYVQERTISFKDDGTYKTRAE |
| sequence | | VKFEGDTLVNRIELKGIDFKEDGNILGHKLEYNFNSHN |
| | | VYITADKQKNGIKANFKIRHNVEDGSVQLADHYQQN |
| | | TPIGDGPVLLPDNHYLSHQSALSKDPNEKRDHMVLLE |
| | | FVTAA |

### Single-Celled Photosynthetic Organisms

Oleaginous organisms that may find use in biofuel production include algae, yeast, filamentous fungi, bacteria (including cyanobacteria), and thraustochytrids. The invention relates in particular to engineered single-celled photosynthetic organisms such as algae and cyanobacteria.

Typically, the organism is single-celled eukaryotic organisms, e.g., a microalgae. Single-celled photosynthetic organism (such as microalgae) are found both in fresh water and salt water. Accordingly, in some embodiments, a single-celled photosynthetic organism for use with the invention is capable of growing in fresh water. In other embodiments, a single-celled photosynthetic organism for use with the invention is capable of growing in salt water

### Algae

Many algal species are single-celled photosynthetic organisms that live in waterbodies. These organisms (commonly referred to as microalgae) are amongst the most efficient photosynthesisers. They have evolved for millions of years to be very good at making fatty acids and lipids from sunlight and CO₂ and then storing then inside the cell.

Accordingly, in some embodiments, a single-celled photosynthetic organism for use with the invention is an algae. In some embodiments, a single-celled photosynthetic organism for use with the invention is a green algae. In some embodiments, a single-celled photosynthetic organism for use with the invention is a brown algae. In some embodiments, a single-celled photosynthetic organism for use with the invention is a red algae. The algae may be selected from any of Chlorophyta, Phaeophyta, Rhodophyta, Xanthophyta, Chrysophyta, Bacillariophyta, Cryptophyta, Dinophyta, Chlorophyta, Euglenophyta, Cyanophyta and Myxophyta.

Particularly suitable for use with the invention are oleaginous algae. Examples of oleaginous algae include Tetraselmis elliptica, Auxenochlorella protothecoides, Botryococcus braunii, Chlorella minutissima, Nannochloropsis salina, Alexandrium sanguinea, Fistulifera solaris, Nitzschia laevis, Chlorella sorokiniana and Chlamydomonas reinhardtii.

In some embodiments, an oleaginous algae can be identified by culturing the organism and determining the percentage of fatty material per dry weight of the resulting culture. For example, to determine the percentage of fatty material per dry weight of a culture, a sample of the culture may be collected and subjected to centrifugation. The resulting cell pellet may be dried (e.g., using an oven). The weight of the dried pellet is then determined. Subsequently, the cells are broken open (e.g., using mortar and pestle, or a cell homogenizer). The cell debris is removed (e.g., by precipitation and centrifugation). The fatty material within the resulting cell-free solution (supernatant) can then be quantitated using gas chromatography-flame ionization detection (GC-FID) or gas chromatography-mass spectrometry (GC-MS). The result can be used to calculate the percentage of fatty material per dry weight of the culture.

In some embodiments, an oleaginous algae suitable for use with the invention comprises at least 15% fatty acid material per dry weight. In some embodiments, an oleaginous algae suitable for use with the invention comprises at least 20% of fatty acid material per dry weight. In some embodiments, an oleaginous algae suitable for use with the invention comprises at least 25% of fatty acid material per dry weight. In some embodiments, an oleaginous algae suitable for use with the invention comprises at least 30% of fatty acid material per dry weight.

In some embodiments, an algae for use with the invention has previously been used in industrial applications, e.g., in biofuel production. Such industrially relevant algae include Chlorella, Dunaliella, Haematococcus, Phaeodactylum, Tetraselmis, Isochrysis, Diacronena, Schizochytrium, Thraustochytrium, Nannochloris, Nannochloropsis, Microchloropsis, Porphyridium, Nanofrustulum, Cryptheiconidium, Schenedesmus, Euglena, Auxenochlorella, Botryococcus, Alexandrium, Fistulifera, and Nitzschia. Exemplary preferred algae are Chlorella sorokiniana, Chlorella vulgaris, Chlorella protothecoides, Dunaliella salina, Dunaliella tertiolecta, Dunaliella sp., Haematococcus pluvialis, Phaeodactylum tricornutum, Tetraselmis suecica, Tetraselmis chuii, Isochrysis galbana, Diacronena volkianum, Schizochytrium sp. Thraustochytrium sp., Nannochloris sp. Nannochloropsis sp., Nannochloropsis gaditana, Porphyridium sp., Nanofrustulum sp., Cryptheiconidium cohnii, Scenedesmus sp., Euglena gracilis, Tetraselmis elliptica, Auxenochlorella protothecoides, Botryococcus braunii, Chlorella minutissima, Nannochloropsis salina, Alexandrium sanguinea, Fistulifera solaris, and Nitzschia laevis.

Particularly suitable algae species for biofuel production include Chlorella, Tetraselmis, Nannochloropsis, Phaeodactylum and Porphyridium.

### Genetic Engineering of Single-Celled Photosynthetic Organisms

A single-celled photosynthetic organism is transformed with an exogenous nucleic acid in order to generate an engineered organisms in accordance with the invention. Suitable methods of transformation are known in the art. Representative methods for generating engineered single-celled photosynthetic organisms in accordance with the invention are described in Example 4.

Following transformation, organisms comprising the exogenous nucleic acid sequence may be selected. In some embodiments, a selection marker included in the nucleic acid is used to select transformants. In other embodiments, a fluorescent marker included in the nucleic acid is used to select transformants.

The engineered organism of the invention may comprise one or more exogenous nucleic acids comprising a coding sequence for a fatty acid transporter operationally linked to a promoter sequence. In some embodiments, the organism may comprise two or more exogenous nucleic acids comprising coding sequences for the same fatty acid transporter. In other embodiments, the organism may comprise two or more exogenous nucleic acids comprising coding sequences for two or more different fatty acid transporters.

### Biosynthetic Pathway Optimisation

An engineered single-celled photosynthetic organism for use with the invention may include one or more additional genetic modifications to increase the amount of free fatty acids inside the cell. Suitable genetic modification are described in, e.g., US patent application publication nos. US 2013/245339A1, US 2013/005003A1 and US 2014/213826A1, each of which is incorporated herewith by reference.

In some embodiments, an engineered single-celled photosynthetic organism for use with the invention comprises one or more endogenous genes that are modified to overproduce fatty acids with a carbon chain length of C6 to C22. In some embodiments, the one or more endogenous genes are selected from the group consisting of an acyl-acyl carrier protein (acyl-ACP) thioesterase, an acetyl-CoA carboxylase, a pyruvate dehydrogenase, acyl-CoA, synthase, an acyl carrier protein, an acyl carrier protein synthase, an acyl-CoA reductase, a decarboxylase, an aldehyde decarbonylasea triglyceride lipase, alkane deformylative monooxygenase, and a carboxylic acid reductase.

In some embodiments, the engineered single-celled photosynthetic organism for use with the invention comprises one or more exogenous genes to overproduce one or more fatty acids with a carbon chain length of C6 to C22. In some embodiments, the one or more exogenous genes are selected from the group consisting of an acyl-acyl carrier protein (acyl-ACP) thioesterase, an acetyl-CoA carboxylase, a pyruvate dehydrogenase, acyl-CoA, synthase, an acyl carrier protein, an acyl carrier protein synthase, an acyl-CoA reductase, a decarboxylase, an aldehyde decarbonylasea triglyceride lipase, alkane deformylative monooxygenase, and a carboxylic acid reductase.

In some embodiments, the one or more fatty acids that are overproduced by an engineered single-celled photosynthetic organism for use with the invention have a carbon chain length of C14 to C22. In some embodiments, the one or more fatty acids have a carbon chain length of C16 to C18 and are optionally monounsaturated. In some embodiments, the one or more fatty acids are palmitoleic acid and/or oleic acid.

The accumulation of free fatty acid may depend on the acyl-ACP thioesterase that is expressed by the single-celled photosynthetic organism (see, e.g., Zhang et al., Metab Eng. 2011; 13(6):713-2, which is incorporated herewith by reference). For example, it has been shown that an Escherichia coli strain carrying an acyl-ACP thioesterase gene from Diploknema butyracea can produce approximately 0.2 g/L of free fatty acid over 48 hours. The same strain carrying an acyl-ACP thioesterase genes from Ricinus communis or Jatropha curcas can produce more than 2.0 g/L over 48 hours. Escherichia coli strains carrying an acyl-ACP thioesterase genes from Ricinus communis or Jatropha curcas can accumulate three major straight-chain free fatty acids, C14, C16:1 and C16.

Accordingly, in some embodiments, an engineered single-celled photosynthetic organism of the invention further comprises an exogenous nucleic acid sequence comprising a coding sequence for an acyl-acyl carrier protein (ACP) thioesterase. In some embodiments, the acyl-ACP thioesterase preferentially liberates a fatty acid with a carbon chain length of C6 to C22. In some embodiments, the acyl-ACP thioesterase preferentially liberates a fatty acid with a carbon chain length of C14 to C22. In some embodiments, the acyl-ACP thioesterase preferentially liberates a fatty acid with a carbon chain length of C16 to C18.

In some embodiments, the acyl-ACP thioesterase is oleoyl-ACP thioesterase. In some embodiments, the oleoyl-ACP thioesterase is a fatA thioesterase. In some embodiments, the fatA thioesterase is from a bacterium or a plant. In some embodiments, the fatA thioesterase is derived from Escherichia coli, Diploknema butyracea, Gossypium hirsutum, Ricinus communis, Jatropha curcas, Helianthus annuus, Umbellularia californica, Arabidopsis thaliana, Brassica juncea or Cuphea hookeriana. In some embodiments, the fatA thioesterase is from a microalga.

In some embodiments, the acyl-ACP thioesterase is palmitoyl-ACP thioesterase. In some embodiments, the palmitoyl-ACP thioesterase is a fatB thioesterase. In some embodiments, the fatB thioesterase is from a bacterium or a plant. In some embodiments, the fatB thioesterase is derived from Eschericha co/i, Diploknema butyracea, Gossypium hirsutum, Ricinus communis, Jatropha curcas, Helianthus annuus, Umbellularia californica, Arabidopsis thaliana, Brassica juncea or Cuphea hookeriana. In some embodiments, the fatB thioesterase is from a microalga.

In some embodiments, an engineered single-celled photosynthetic organism of the invention further comprises a first exogenous nucleic acid sequence comprising a coding sequence for oleoyl-ACP thioesterase (e.g., a bacterial or plant fatA thioesterase) and a second exogenous nucleic acid sequence comprising a coding sequence for palmitoyl-ACP thioesterase (e.g., a bacterial or plant fatB thioesterase).

### Cultures

In some embodiments, the invention relates to a culture comprising an engineered organism described herein. Typically, a culture comprises a plurality of organisms of the same genetic make-up suspended in a suitable medium, e.g., a culture medium suitable for supporting growth or survival of the engineered organisms. In some embodiments, the culture medium comprises an agent (e.g., an antibiotic) for which the exogenous nucleic acid comprised in the engineered organism includes a selection marker (e.g., a nucleic acid sequence that is capable of providing resistance to the antibiotic).

The engineered single-celled photosynthetic organisms described herein may be able to grow in a suitable liquid medium. For example, a liquid medium such as water (e.g., rain water) comprising an inorganic carbon source (typically CO₂) and exposed to sun light may be sufficient to support the growth of the engineered single-celled photosynthetic organisms described herein.

In some embodiments, the medium may be enriched with an inorganic carbon source (typically CO₂), e.g., it may comprise CO₂ at concentrations that are higher than atmospheric CO₂ levels (about 0.04% by volume). For example, the medium may be enriched with CO₂ by bubbling CO₂ gas into the medium. In some embodiments, the medium comprises about 1% by volume CO₂. In other embodiments, medium comprises at least 1% by volume CO₂. In some embodiments, the medium comprises about 2%. by volume CO₂. In other embodiments, medium comprises at least 2% by volume CO₂.

### Methods of Producing Fatty Acids

The invention is also directed to methods of producing fatty acids that employ the engineered single-celled photosynthetic organisms described herein. The method of producing fatty acids comprises culturing an engineered organism of the invention in a medium suitable for growing the organism.

In some embodiments, a culture medium inoculated with the engineered organism of the invention is incubated for a period of time sufficient to yield fatty acids (e.g., in the culture medium). In some embodiments, the incubation period is 1-7 days. In some embodiments, the incubation period is 1 day, 2 days, 3 days, 4 days, 5 days, 6 days, 7 days, 2 weeks, 3 weeks, 1 month, 2 months, or 3 months. Methods for culturing single-celled photosynthetic organisms are known in the art.

A method of producing fatty acids typically comprises exposing an engineered organism of the invention to light and an inorganic carbon source (typically CO₂), wherein the exposure to light results in the conversion of the inorganic carbon source by the engineered organisms into fatty acids. CO₂ readily dissolves in water, in which it can take the form of carbonic acid (H₂CO₃), bicarbonate (HCO₃⁻), and carbonate (CO₃⁻).

In some embodiments, the culture medium is supplemented with a low-CO₂ emission liquid carbon source such as acetate (obtainable directly from primary photosynthesis).

As demonstrated in Example 6, fatty acids produced by an engineered organism of the invention are readily secreted into the culture medium by a fatty acid transporter encoded by an exogenous nucleic acid of the invention. The culture medium comprising the fatty acids may then be separated from the engineered organism. Typically, the organism is not disrupted or otherwise damaged to obtain the fatty acids.

A method of producing fatty acids in accordance with the invention may be conducted at a culture volume for laboratory scale (less than 2.5 L). It may also be scaled up to culture volumes for pilot plant scale (2.5-25 L) and commercial scale (more than 25 L), for the industrial production of fatty acids, typically for use in biofuels.

The method of producing fatty acids in accordance with the invention may produce at least 1 μg fatty acid per litre (L) of culture medium per day (d). In some embodiments, a method in accordance with the invention produces fatty acid at a rate of at least 10 μg/L/d, at least 20 μg/L/d, at least 30 μg/L/d, at least 40 μg/L/d, at least 50 μg/L/d, at least 60 μg/L/d, at least 70 μg/L/d, at least 80 μg/L/d, at least 90 μg/L/d, at least 100 μg/L/d, at least 200 μg/L/d, at least 300 μg/L/d, at least 400 μg/L/d, at least 500 μg/L/d, at least 600 μg/L/d, at least 700 μg/L/d, at least 800 μg/L/d, at least 900 μg/L/d, or at least 1 g/L/d.

A culture medium inoculated with the engineered organism of the invention and secreting fatty acids may be cultured for at least 1 week. For example, the inoculated culture medium secreting fatty acids may be cultured for at least 2 weeks, at least 3 weeks, at least 1 month, at least 2 months, at least 2 months, at least 4 months or at least 5 months.

In some embodiments, a culture medium comprising an engineered single-celled photosynthetic organism in accordance with the invention is enriched for monounsaturated fatty acids having a carbon chain length of C16 to C18 relative to a culture medium of a corresponding wild-type single-celled photosynthetic organism incubated under the same conditions. In some embodiments, the culture medium comprises at least about 5-fold (e.g., at least about 10-fold) more palmitoleic acid (C16:1) after incubation with an engineered single-celled photosynthetic organism of the invention than a culture medium of a corresponding wild-type single-celled photosynthetic organism incubated under the same conditions. In some embodiments, the culture medium comprises at least about 1.5-fold (e.g., at least about 3-fold or at least about 5-fold) more oleic acid (C18:1) after incubation with an engineered single-celled photosynthetic organism of the invention than a culture medium of a corresponding wild-type single-celled photosynthetic organism incubated under the same conditions.

In some embodiments, culturing of an engineered organisms of the invention is continuous at a steady state. In these embodiments, culture medium comprising secreted fatty acids is separated from the organism and replaced with new culture medium without interrupting culture of the organism.

### Separation of Fatty Acids

A method of producing fatty acids in accordance with the invention may further comprise a step of separating the culture medium from the organism. In some embodiments, the step of separating the medium from the organism comprises sedimentation. Sedimentation may include centrifugation of the medium. In some embodiments, sedimentation occurs naturally (e.g., by incubating the medium without agitation). In other embodiments, the step of separating the medium from the organism may comprise filtration. Typically, separation of the medium from the organism does not damage (e.g., disrupt) the organism.

Both sedimentation (e.g., by centrifugation or by incubating the medium without agitation) and filtration may be used to obtain an organism-free culture medium. In some embodiments, the organism-free culture medium may be subjected to a liquid-liquid extraction in order to isolate the fatty acids from the organism-free culture medium. In other embodiments, the fatty acids are collected from the organism-free culture medium, e.g., by spooning droplets comprising the fatty acids from the surface of the organism-free culture medium. Spooning may be performed with an automated mechanical device.

For example, a culture medium after having been inoculated and subsequently incubated with an engineered organism of the invention for a sufficient period of time may comprise at least 1 μg fatty acid per litre of culture medium. In some embodiments, the culture medium comprises at least 10 μg fatty acid per litre of culture medium. In some embodiments, the culture medium comprises at least 100 μg fatty acid per litre of culture medium. In some embodiments, the culture medium comprises at least 1 g fatty acid per litre of culture medium.

The extracted fatty acids may be subjected to further processing steps, e.g., to obtain a product suitable for use a biofuel. The further processing, e.g., for biofuel production, may include transesterification, decarboxylation or hydrocracking (also referred also hydroprocessing).

### Downstream Applications

Fatty acids obtainable from the engineered single-celled photosynthetic organisms described herein may be used (either directly or after further purification) in cosmetics, pharmaceuticals, chemicals, and neutraceuticals. Alternatively, the may be processed further for use in biofuels.

### Biofuel Production

In some embodiments, fatty acids obtainable from the engineered single-celled photosynthetic organisms described herein may be subjected to a transesterification reaction with methanol to produce a biofuel. For example, the resulting methyl-transesterified fatty acids may be used directly as a biofuel. In some embodiments, the resulting methyl-transesterified fatty acids are blended with a fossil fuel (e.g., diesel fuel).

In some embodiments, fatty acids obtainable from the engineered single-celled photosynthetic organisms described herein may be subjected to a decarboxylation reaction. In some embodiments, decarboxylation comprises the addition of hydrogen (H₂) and a metal catalyst (typically at high pressure). In other embodiments, decarboxylation is performed enzymatically. The decarboxylation reaction produces an alkane or alkene with an unbranched aliphatic chain that is one carbon atom shorter than the aliphatic chain of the fatty acid from which it derives.

In some embodiments, fatty acids obtainable from the engineered single-celled photosynthetic organisms described herein are treated at high temperature and pressure by the addition of hydrogen and water (“hydrocracking”) to produce a range of alkanes and alkenes of different aliphatic chain lengths. The resulting hydrogenated vegetable oil (HVO) or hydroprocessed esters and fatty acid (HEFA) may be used as renewable drop-in fuel. They do not require blending with fossil fuel.

### NUMBERED EMBODIMENTS

1. An engineered single-celled photosynthetic organism comprising an exogenous nucleic acid sequence comprising a coding sequence for a fatty acid transporter operationally linked to a promoter sequence.
2. The engineered organism of embodiment 1, wherein the fatty acid transporter is localised in the cytoplasmic membrane of the organism upon expression.
3. The engineered organism of embodiment 1 or embodiment 2, wherein the fatty acid transporter is capable of transporting fatty acids with a carbon chain length of C6 to C22 across the cytoplasmic membrane of the organism from inside the cell.
4. The engineered organism of any preceding embodiment, wherein the fatty acid transporter is capable of transporting fatty acids with a carbon chain length of C14 to C22 across the cytoplasmic membrane of the organism from inside the cell.
5. The engineered organism of any preceding embodiment, wherein the fatty acid transporter is capable of transporting fatty acids with a carbon chain length of C16 to C18 across the cytoplasmic membrane of the organism from inside the cell.
6. The engineered organism of any preceding embodiment, wherein the fatty acid transporter is capable of transporting fatty acids with a carbon chain length of C16 and/or C18 across the cytoplasmic membrane of the organism from inside the cell.
7. The engineered organism of embodiment 6, wherein the fatty acids are monounsaturated.
8. The engineered organism of any preceding embodiment, wherein the fatty acid transporter is capable of transporting palmitoleic acid and/or oleic acid across the cytoplasmic membrane of the organism from inside the cell.
9. The engineered organism of any preceding embodiment, wherein the coding sequence for the fatty acid transporter is derived from the genome of a plant cell or a mammalian cell.
10. The engineered organism of embodiment 9, wherein the coding sequence for the fatty acid transporter is derived from the genome of a plant cell.
11. The engineered organism of embodiment 10, wherein the fatty acid transporter is an ABC transporter.
12. The engineered organism of embodiment 11, wherein the ABC transporter is the Arabidopsis thaliana ABCG11 protein or a functional homolog thereof.
13. The engineered organism of embodiment 11, wherein the ABC transporter is the Oryza sativa ABCG15 protein or a functional homolog thereof.
14. The engineered organism of embodiment 9, wherein the coding sequence for the fatty acid transporter is derived from the genome of a mammalian cell.
15. The engineered organism of embodiment 14, wherein the fatty acid transporter is an ABC transporter or flippase.
16. The engineered organism of embodiment 15, wherein the flippase is the Homo sapiens FATP1 protein or a functional homolog thereof.
17. The engineered organism of any of preceding embodiment, wherein the promoter sequence is derived from the genome of an organism that is different from the genome from which the coding sequence is derived.
18. The engineered organism of embodiment 17, wherein the promoter sequence is exogenous to the engineered organism.
19. The engineered organism of embodiment 17, wherein the promoter sequence is endogenous to the engineered organism.
20. The engineered organism of embodiment 17 or 18, wherein the promoter is selected from the group consisting of the cauliflower mosaic virus (CaMV) 35S promoter, a Nitrogen Reductase (NR) promoter, a Photosystem I reaction center Subunit II (PSAD) promoter, and a HSP70A-RBCS2 (AR) promoter.
21. The engineered organism of any preceding embodiment, wherein the coding sequence is codon-optimised for expression in the engineered organism.
22. The engineered organism of any preceding embodiment, wherein the coding sequence comprises one or more introns.
23. The engineered organism of any preceding embodiment, wherein the exogenous nucleic acid sequence further comprises a selection marker.
24. The engineered organism of embodiment 23, wherein the selection marker provides antibiotic resistance.
25. The engineered organism of any preceding embodiment, wherein the exogenous nucleic acid sequence further comprises a terminator sequence operationally linked to the coding sequence.
26. The engineered organism of embodiment 25, wherein the terminator sequence is from the genome of an organism that is different from the genome from which the coding sequence and/or the promoter sequence is/are derived.
27. The engineered organism of embodiment 26, wherein the terminator sequence is an exogenous to the engineered organism.
28. The engineered organism of embodiment 26, wherein the terminator sequence is endogenous to the engineered organism.
29. The engineered organism of any one of embodiments 25-27, wherein the terminator sequence encodes an Agrobacterium tumefaciens nopaline synthase (NOS) terminator.
30. The engineered organism of any preceding embodiment, wherein the organism is an algae.
31. The engineered organism of embodiment 30, wherein the algae is an oleaginous algae.
32. The engineered organism of any preceding embodiment, wherein the organism is capable of growing in fresh water.
33. The engineered organism of any one of embodiments 1 to 31, wherein the organism is capable of growing in salt water.
34. The engineered organism of embodiment 30, wherein the organism is an algae selected from Chlorophyta, Phaeophyta, Rhodophyta, Xanthophyta, Chrysophyta, Bacillariophyta, Cryptophyta, Dinophyta, Euglenophyta, Cyanophyta and Myxophyta.
35. The engineered organism of embodiment 34, wherein the organism is selected from the group consisting of Chlorella, Chlamydomonas, Dunaliella, Haematococcus, Phaeodactylum, Tetraselmis, Isochrysis, Diacronena, Schizochytrium, Thraustochytrium, Nannochloris, Nannochloropsis, Microchloropsis, Porphyridium, Nanofrustulum, Cryptheiconidium, Schenedesmus, Euglena, Auxenochlorella, Botryococcus, Alexandrium, Fistulifera and Nitzschia.
36. The engineered organism of embodiment 35, wherein the organism is selected from the group consisting of Chlorella sorokiniana, Chlorella vulgaris, Chlorella protothecoides, Chlamydomonas reinhardtii, Dunaliella salina, Dunaliella tertiolecta, Dunaliella sp., Haematococcus pluvialis, Phaeodactylum tricornutum, Tetraselmis suecica, Tetraselmis chuii, Isochrysis galbana, Diacronena volkianum, Schizochytrium sp. Thraustochytrium sp., Nannochloris sp. Nannochloropsis sp., Nannochloropsis gaditana, Porphyridium sp., Nanofrustulum sp., Cryptheiconidium cohnii, Scenedesmus sp., Euglena gracilis, Tetraselmis elliptica, Auxenochlorella protothecoides, Botryococcus braunii, Chlorella minutissima, Nannochloropsis salina, Alexandrium sanguinea, Fistulifera solaris, and Nitzschia laevis.
37. The engineered organism of any one of the preceding embodiments, wherein the engineered organism comprises one or more endogenous genes that are modified to overproduce fatty acids with a carbon chain length of C6 to C22.
38. The engineered organism of embodiment 37, wherein the one or more endogenous genes are selected from the group consisting of an acyl-acyl carrier protein (acyl-ACP) thioesterase, an acetyl-CoA carboxylase, a pyruvate dehydrogenase, acyl-CoA, synthase, an acyl carrier protein, an acyl carrier protein synthase, an acyl-CoA reductase, a decarboxylase, an aldehyde decarbonylasea triglyceride lipase, alkane deformylative monooxygenase, and a carboxylic acid reductase.
39. The engineered organism of any one of the preceding embodiments, wherein the engineered organism comprises one or more exogenous genes to overproduce one or more fatty acids with a carbon chain length of C6 to C22.
40. The engineered organism of embodiment 39, wherein the one or more exogenous genes are selected from the group consisting of an acyl-acyl carrier protein (acyl-ACP) thioesterase, an acetyl-CoA carboxylase, a pyruvate dehydrogenase, acyl-CoA, synthase, an acyl carrier protein, an acyl carrier protein synthase, an acyl-CoA reductase, a decarboxylase, an aldehyde decarbonylase triglyceride lipase, alkane deformylative monooxygenase, and a carboxylic acid reductase.
41. The engineered organism of any one of embodiments 37-40, wherein the one or more fatty acids have a carbon chain length of C14 to C22.
42. The engineered organism of any one of embodiments 37-40, wherein the one or more fatty acids have a carbon chain length of C16 to C18 and optionally are monounsaturated.
43. The engineered organism of any one of embodiments 37-40, wherein the one or more fatty acids are palmitoleic acid and/or oleic acid.
44. The engineered organism of any one of embodiments 37-40, further comprising an exogenous nucleic acid sequence comprising a coding sequence for an acyl-acyl carrier protein (ACP) thioesterase.
45. The acyl-ACP thioesterase of embodiment 44, wherein the acyl-ACP thioesterase preferentially liberates a fatty acid with a carbon chain length of C6 to C22.
46. The acyl-ACP thioesterase of embodiment 44, wherein the acyl-ACP thioesterase preferentially liberates a fatty acid with a carbon chain length of C14 to C22.
47. The acyl-ACP thioesterase of embodiment 44, wherein the acyl-ACP thioesterase preferentially liberates a fatty acid with a carbon chain length of C16 to C18.
48. The acyl-ACP of any one of embodiments 44-47, wherein the acyl-ACP is oleoyl-ACP thioesterase, optionally wherein the oleoyl-ACP is a fatA thioesterase.
49. The fatA thioesterase of embodiment 48, wherein the fatA thioesterase is derived from a bacterium or a plant, optionally (a) Escherichia coli, Diploknema butyracea, Gossypium hirsutum, Ricinus communis, Jatropha curcas, Helianthus annuus, Umbellularia californica, Arabidopsis thaliana, Brassica juncea or Cuphea hookeriana; or (b) a microalga.
50. The acyl-ACP of any one of embodiments 44-47, wherein the acyl-ACP is palmitoyl-ACP thioesterase, optionally wherein the palmitoyl-ACP is a fatB thioesterase.
51. The fatB thioesterase of embodiment 50, wherein the fatB thioesterase is derived from a bacterium or a plant, optionally (a) Escherichia coli, Diploknema butyracea, Gossypium hirsutum, Ricinus communis, Jatropha curcas, Helianthus annuus, Umbellularia californica, Arabidopsis thaliana, Brassica juncea or Cuphea hookeriana; or (b) a microalga.
52. The engineered organism of any one of embodiments 37-40, further comprising a first exogenous nucleic acid sequence comprising a coding sequence for oleoyl-ACP thioesterase and a second exogenous nucleic acid sequence comprising a coding sequence for palmitoyl-ACP thioesterase.
53. The exogenous nucleic acid sequences of embodiment 52, wherein (a) the oleoyl-ACP thioesterase is a bacterial or plant fatA thioesterase and/or (b) the palmitoyl-ACP thioesterase is a bacterial or plant fatB thioesterase.
54. A nucleic acid comprising a coding sequence for a fatty acid transporter operationally linked to a promoter sequence.
55. The nucleic acid of embodiment 54, wherein the fatty acid transporter is capable of transporting fatty acids with a carbon chain length of C6 to C22 across the cytoplasmic membrane of the organism from inside the cell.
56. The nucleic acid of embodiment 54 or 55, wherein the fatty acid transporter is capable of transporting fatty acids with a carbon chain length of C14 to C22 across the cytoplasmic membrane of the organism from inside the cell.
57. The nucleic acid of any one of embodiments 54-56, wherein the fatty acid transporter is capable of transporting fatty acids with a carbon chain length of C16 to C18 across the cytoplasmic membrane of the organism from inside the cell.
58. The nucleic acid of any one of embodiments 54-57, wherein the fatty acid transporter is capable of transporting fatty acids with a carbon chain length of C16 and/or C18 across they cytoplasmic membrane of the organism from inside the cell.
59. The nucleic acid of embodiment 58, wherein the fatty acids are monounsaturated.
60. The nucleic acid of any one of embodiments 54-59, wherein the fatty acid transporter is capable of transporting palmitoleic acid and/or oleic acid across the cytoplasmic membrane of the organism from inside the cell.
61. The nucleic acid of any of embodiments 54-60, wherein the coding sequence for the fatty acid transporter is derived from the genome a plant cell or a mammalian cell.
62. The nucleic acid of embodiment 61, wherein the coding sequence for the fatty acid transporter is derived from the genome of a plant cell.
63. The nucleic acid of embodiment 62, wherein the coding sequence for the fatty acid transporter encodes an ATP-binding cassette (ABC) transporter.
64. The nucleic acid of embodiment 63, wherein the ABC transporter is the Arabidopsis thaliana ABCG11 protein or a functional homolog thereof.
65. The nucleic acid of embodiment 63, wherein the ABC transporter is the Oryza sativa ABCG15 protein or a functional homolog thereof.
66. The nucleic acid of embodiment 61, wherein the coding sequence for the fatty acid transporter is derived from the genome of a mammalian cell.
67. The nucleic acid of embodiment 66, wherein the coding sequence for the fatty acid transporter encodes an ABC transporter or a flippase.
68. The nucleic acid of embodiment 67, wherein the coding sequence for the fatty acid transporter encodes the Homo sapiens flippase FATP1 protein or a functional homolog thereof.
69. The nucleic acid of any of embodiments 54-68, wherein the promoter sequence is derived from the genome of an organism that is different from the genome from which the coding sequence is derived.
70. The nucleic acid of any of embodiments 54-69, wherein the promoter is selected from the group consisting of the cauliflower mosaic virus (CaMV) 35S promoter, a Nitrogen Reductase (NR) promoter, a Photosystem I reaction center Subunit II (PSAD) promoter, and a HSP70A-RBCS2 (AR) promoter.
71. The nucleic acid of any of embodiments 54-70, wherein the promoter is derived from a different organism to the fatty acid transporter.
72. The nucleic acid of any of embodiments 54-71, wherein the fatty acid transporter nucleic acid sequence is codon-optimised.
73. The nucleic acid of any of embodiments 54-72, further comprising one or more introns.
74. The nucleic acid of any of embodiments 54-73, further comprising a selection marker.
75. The nucleic acid of embodiment 74, wherein the selection marker provides antibiotic resistance.
76. The nucleic acid of any of embodiments 54-75, further comprising a terminator sequence operationally linked to the coding sequence.
77. The nucleic acid of embodiment 76, wherein the terminator sequence is derived from the genome of an organism that is different from the genome from which the coding sequence and/or the promoter sequence is/are derived.
78. The nucleic acid of embodiment 76 or embodiment 67, wherein the terminator is the Agrobacterium tumefaciens nopaline synthase (NOS) terminator.
79. A culture comprising the organism of any of embodiments 1-53.
80. A method for producing fatty acids comprising culturing the organism of any of embodiments 1-53 in a medium suitable for growing the organism.
81. The method of embodiment 80, wherein culturing is continuous at a steady state.
82. The method of embodiment 80 or 81, further comprising a step of separating the medium from the organism.
83. The method of embodiment 82, wherein the step of separating comprises sedimentation or filtration.
84. The method of embodiment 83, wherein sedimentation involves centrifugation.
85. The method of embodiment 83, wherein sedimentation involves incubating the medium without agitation for a period of time.
86. The method of any one of embodiments 83-85, further comprising a step of extracting the fatty acids from the organism-free medium obtained by sedimentation or filtration using a liquid-liquid extraction process.
87. The method of any one of embodiments 83-85, further comprising spooning droplets comprising the fatty acids from the surface of the organism-free culture medium obtained by sedimentation or filtration to extract the fatty acids.
88. The method of embodiment 86 or 87, further comprising processing the extracted fatty acids by transesterification.
89. The method of embodiment 86 or 87, further comprising processing the extracted fatty acids by decarboxylation.
90. The method of embodiment 86 or 87, further comprising processing the extracted fatty acids by hydrocracking.
91. The method of any one of embodiments 80-90, wherein the method produces at least 1 μg fatty acid per litre of culture medium per day.
92. The method of embodiment 91, wherein the method produces at least 10 μg fatty acid per litre of culture medium per day.
93. The method of embodiment 92, wherein the method produces at least 100 μg fatty acid per litre of culture medium per day.
94. The method of embodiment 93, wherein the method produces at least 1 g fatty acid per litre of culture medium per day.
95. A culture medium inoculated with the engineered organism of any of embodiments 1-53 and incubated for a period of time sufficient to yield at least 1 μg fatty acid per litre of culture medium.
96. The culture medium of embodiment 95, incubated for a period of time sufficient to yield at least 10 μg fatty acid per litre of culture medium.
97. The culture medium of embodiment 96, incubated for a period of time sufficient to yield at least 100 μg fatty acid per litre of culture medium.
98. The culture medium of embodiment 97, incubated for a period of time sufficient to yield at least 1 g fatty acid per litre of culture medium.
99. The culture medium of any one of embodiments 95-98, wherein the culture medium is free of the organism used for inoculation.
100. A method of extracting fatty acids from a single-celled photosynthetic organism, comprising:
a. providing the culture medium of any one of embodiments 95-99;
b. extracting the fatty acids.
101. The method of embodiment 100, wherein the step of extracting comprises a liquid-liquid extraction process.
102. The method of embodiment 100, wherein the step of extracting comprises spooning droplets comprising the fatty acids from the surface of the culture medium.
103. A method of producing a biofuel, comprising:
a. providing a fatty acid obtained by the method of any one of embodiment 80-87 and 90-92; and
b. processing the fatty acid by transesterification, decarboxylation or hydrocracking.
104. A biofuel obtained by the method of embodiment 103.
105. A culture medium conditioned by an engineered single-celled photosynthetic organism of any of embodiments 1-53, wherein the culture medium is enriched for monounsaturated fatty acids having a carbon chain length of C16 to C18 relative to a culture medium condition by a corresponding wild-type single-celled photosynthetic organism under the same conditions.
106. The culture medium of embodiment 105, wherein the culture medium comprises at least about 5-fold more palmitoleic acid (C16:1) than a culture medium conditioned by a corresponding wild-type single-celled photosynthetic organism under the same conditions.
107. The culture medium of embodiment 105, wherein the culture medium comprises at least about 1.5-fold more oleic acid (C18:1) than a culture medium conditioned by a corresponding wild-type single-celled photosynthetic organism under the same conditions.
108. A cell-free fatty acid composition secreted by an engineered single-celled photosynthetic organism of any of embodiments 1-53, wherein the composition is enriched for monounsaturated fatty acids having a carbon chain length of C16 to C18 relative to a composition secreted by a corresponding wild-type single-celled photosynthetic organism under the same conditions.
109. The composition of embodiment 108, wherein the composition comprises at least about 5-fold more palmitoleic acid (C16:1) than a composition secreted by a corresponding wild-type single-celled photosynthetic organism under the same conditions.
110. The composition of embodiment 108, wherein the composition comprises at least about 1.5-fold more oleic acid (C18:1) than a composition secreted by a corresponding wild-type single-celled photosynthetic organism under the same conditions.

### Examples

The following examples are included for illustrative purposes only and are not intended to limit the scope of the invention.

### Example 1. Identification of Plant-Derived ABC Transporters Suitable for the Translocation of Fatty Acids Across the Cytoplasmic Membrane

This example describes the identification of plant-derived ABC transporters suitable for the translocation of fatty acids across the cytoplasmic membrane.

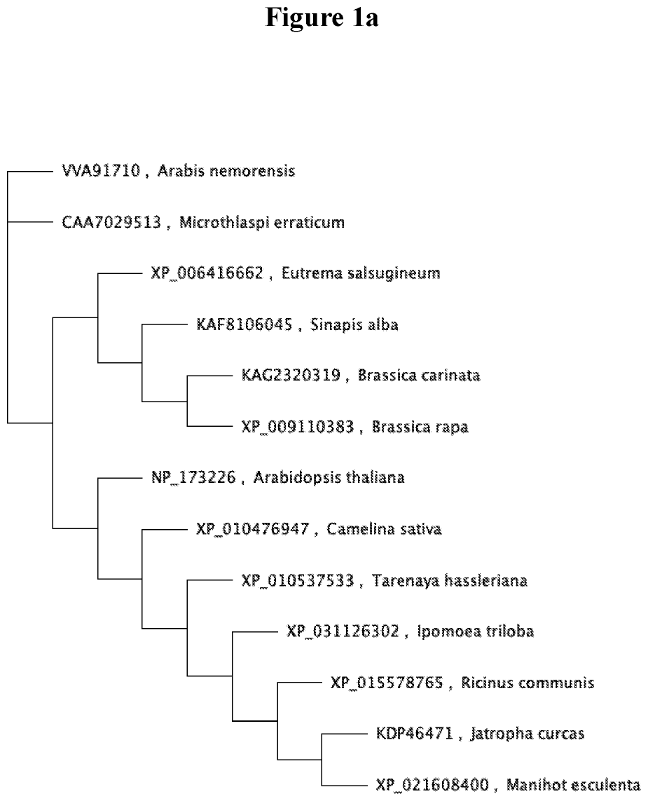

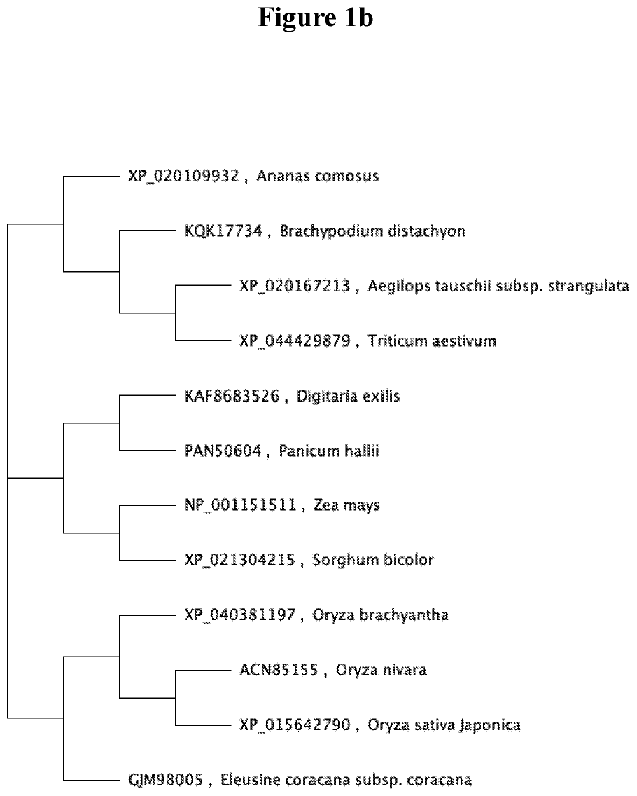

The inventors identified two exemplary ABC transporters, ABCG11 and ABCG15, that are particularly effective in transporting fatty acids across the cytoplasmic membranes of plant cells,. Both ABCG11 and ABCG15 are evolutionarily conserved ABC transporters with many known homologues in the plant kingdom (FIGS. 1a and 1b, respectively).

The Arabidopsis thaliana ABCG11 protein is crucial for the formation of cuticle. Cuticle is the protective wax layer on leaves. It is also crucial for vascular development. The physiological and in vitro activity of the protein itself has never been tested. Without wishing to be bound by any particular theory, the present inventors associated the role in the formation of cuticle with the transport of long-chain fatty acids to the surface of the lead of A. thaliana plants to form the cuticle.

The Oryza sativa ABCG15 is essential for rice pollen exine and sporopollenin development. It has been suggested that the exine and sporopollenin are formed by complex polymerization of long-chain wax and aliphatic cutin polymers (Li et al. The molecular structure of plant sporopollenin, Nature Plants 5, 41-46 (2019). Without wishing to be bound by any particular theory, the inventors consider this to be the likely substrate for ABCG15, so identified ABCG15 as a suitable fatty acid transporter for use with the invention.

Functional homologues to ABCG11 and ABCG15, such as those identified in FIG. 1, are considered to transport fatty acids across the cytoplasmic membrane in a similar manner and with a similar specificity to ABCG11 and ABCG15 and therefore are contemplated as suitable alternatives for use with the invention.

### Example 2. Identification of a Mammalian-Derived Flippase Suitable for the Translocation of Fatty Acids Across the Cytoplasmic Membrane

This example describes the identification of a mammalian-derived flippase suitable for the translocation of fatty acids across the cytoplasmic membrane.

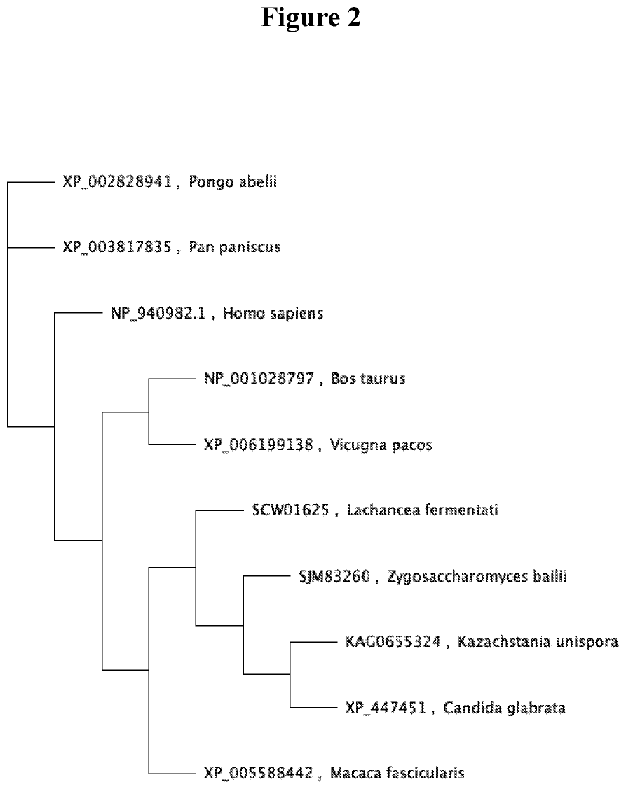

Homo sapiens FATP1 is an exemplary mammalian-derived flippase. It is an evolutionary conserved membrane protein with functional homologues in many mammalian species (FIG. 2). Its function is in maintaining homeostasis of fatty acids, and it is known to act concertedly with the insulin signaling pathway. It only has two transmembrane helices and its function seems to largely occur inside the cell rather than in the cytoplasmic membrane. However, the protein's subcellular localization is under debate and does not appear to be consistent across different cell types. For instance, in 293 cell lines, FATP1 appears to localize to the cytoplasmic membrane, whereas in 3T3-L1 and myocytes it localized to the endoplasmic reticulum and mitochondria, respectively.

In yeast, the FATP1 homologue is associated with the import of long-chain fatty acids (Obermeyer et al. Topology of the yeast fatty acid transport protein Fat1p: mechanistic implications for functional domains on the cytoplasmic surface of the plasma membrane, J. Lipid Res, 2007; 48(11)). In the blood brain barrier of mammalian animals, FATP1 seems to be responsible for the crossing of fatty acid through the blood brain barrier itself by regulating both import and export of fatty acid at a cellular level (Ochiai et al. The blood-brain barrier fatty acid transport protein 1 (FATP1/SLC27A1) supplies docosahexaenoic acid to the brain, and insulin facilitates transport. J Neurochem. 2017 May; 141(3):400-412).

FATP1 and its functional homologues are believed to act by a mechanisms distinct from the plant-derived ABC transporters described in Example 1. In particular, it is thought that they act as flippase aiding the movement of phospholipid molecules between the two leaflets that compose a bilayer membrane through transverse diffusion. Without wishing to be bound by any particular theory, the inventors hypothesize that FATP1 and its homologues can be utilized to transport fatty acids out of single-celled photosynthetic organisms into the surrounding culture medium.

### Example 3. Generation of Expressible and Selectable Nucleic Acids Comprising a Coding Sequence for a Fatty Acid Transporter

This example illustrates the generation of exemplary nucleic acids comprising a coding sequence for a fatty acid transporter operationally linked to a promoter sequence that can be used to express the fatty acid transporter in an engineered single-celled photosynthetic organism.

The ABC transporter genes ABCG11 and ABCG15 and the flippase FATP1 were codon-optimized with the Intronserter open tool to ensure optimal expression in Chlamydomonas (Jaeger et al.). Introns were also inserted into the genes to aid expression. Gene expression was driven with a single copy of the Cauliflower Mosaic Virus (CaMV) 35s promoter and the nucleic acid also encoded the Agrobacterium tumefaciens nopaline synthase (NOS) terminator to generate expression cassettes for each of ABCG11, ABCG15 and FATP1. The expression cassette was coupled to a selection marker cassette of aminoglycoside-3′-adenyltransferase, driven and terminated by a β-tubulin2 promoter and a PSAD terminator native to Chlamydomonas. The selection marker provides antibiotic resistance against spectinomycin.

Separate nucleic acids were created wherein the expression cassette comprised the clover tag after the fatty acid transporter. The cassettes are illustrated in FIG. 3.

### Example 4. Transformation of Nucleic Acids Encoding Fatty Acid Transporters into Single-Celled Photosynthetic Organisms

This example demonstrates successful transformation of an exemplary single-celled photosynthetic organism with the exemplary nucleic acids generated in Example 3.

Chlamydomonas reinhardtii cells were transformed with the nucleic acids prepared in Example 3. C. reinhardtii strain CC-5416 was obtained from the Chlamydomonas Resource Centre, University of Minnesota (Kurniasih et al. (2016) UV-mediated Chlamydomonas mutants with enhanced nuclear transgene expression by disruption of DNA methylation-dependent and independent silencing systems. Plant Mol Biol. 92:629-641).

The method for transformation was adapted from Crozet et al. Birth of a Photosynthetic Chassis: A MoClo Toolkit Enabling Synthetic Biology in the Microalga Chlamydomonas reinhardtii, ACS Synth. Biol., 2018 7 (9), 2074-2086. Cells were inoculated at low density in TAP medium and grown in a wide-bottom Fernback flask with a large magnetic stirrer at 25° C. with a 16/8 illumination light cycle. Cells were resuspended in fresh TAP to cell density of 5×10⁸. 250 μL cells were mixed with 250 ng linear DNA in a 0.4-cm gap electroporation cuvette and placed on ice for 10 minutes. Afterwards, the cuvette was electroporated at 800 V (2 kV/cm), 25 μF, with infinity shunt resistance. Cells were removed and placed in 10 mL of TAP+60 mM sucrose and incubated at 25° C. with gentle shaking in the dark for 16 hours for recovery. Cells were centrifuged at 1500 g for 10 minutes at 4° C., resuspended in 500 μL of TAP+60 mM, and plated on TAP agar with 100 μM of spectinomycin, and incubated with 16/8 illumination light cycle until colonies formed. Colonies were passaged on fresh antibiotic plates for 2 more rounds.

After the three consecutive passages, genomic inserts were confirmed with PCR on genomic DNA extracted from growing culture. Genomic DNA was extracted and purified (Barbier et al. A phenol/chloroform-free method to extract nucleic acids from recalcitrant, woody tropical species for gene expression and sequencing. Plant Methods 15, 62 (2019)). The target of the PCR was the aadA gene, encoding for spectinomycin resistance. Genomic insertion was confirmed using VeriFi polymerase or Repliga Toughmix 2× mastermix. Successful transformation is demonstrated by FIG. 4.

### Example 5. Fatty Acid Transporters Localize to the Cytoplasmic Membrane

This example demonstrates that a nucleic acid sequence of the invention comprising a coding sequence for a fatty acid transporter operationally linked to a promoter sequence is expressed by an exemplary single-celled photosynthetic organisms and localize to the cytoplasmic membrane.

Cells transformed in Example 4 with the nucleic acid coding for ABCG15 comprising the fatty acid clover tag resulted in expression of the exogenous fatty acid transporters fused to the clover tag. Said cells were imaged using fluorescence microscopy with immunofluorescence assay. All fluorescence microscopy was conducted with Leica DMIL with 100 w Osram HBO light source and 100× Leica Fluotar 1.30 oil objective. Microscopic images were taken with an Altair hypercam 183M camera. Scale bars were branded on the images with ImageJ. A liquid blocker marker was used to mark poly-L-lysine glass slides. 100 μl of late log phase cell culture were placed inside the marked area and left to immobilize for 1 hour. Excess liquid was taken off with a pipette and replaced with 150 μL of freshly defrosted 4% paraformaldehyde dissolved in PBS. After fixation, cells were washed twice with PBS, for 5 minutes per wash. The cell membrane was then permeabilised with 150 μL 0.5% saponin dissolved in PBS. Cells were washed twice with PBS and once with 1 mM Tris, each for 5 minutes. Cells were then blocked with 150 μL 2% BSA dissolved in PBS for 30 minutes. Afterwards, primary antibody incubation was performed without washing steps, using anti-GFP (66002-1-Ig, Proteintech) diluted 1:100 in 2% BSA/PBS for 60 minutes. Then cells were washed 3 times with PBS, for 5 minutes per wash. Secondary antibody incubation was performed with a DL488-conjugated secondary body (GtxRb-003-D488NHSX, Immunoreagents) diluted 1:100 in 2% BSA/PBS for 60 minutes. Then cells were washed twice with PBS and twice with milli-Q water, 5 minutes per wash. Slides were then allowed to almost dry while shielded from light, mounted with 5 μL of Prolong Diamond (Thermofisher) and sealed with #1.5 coverslip and nail polish. Slides were then stored at 4° C. overnight and observed under a fluorescence microscope.

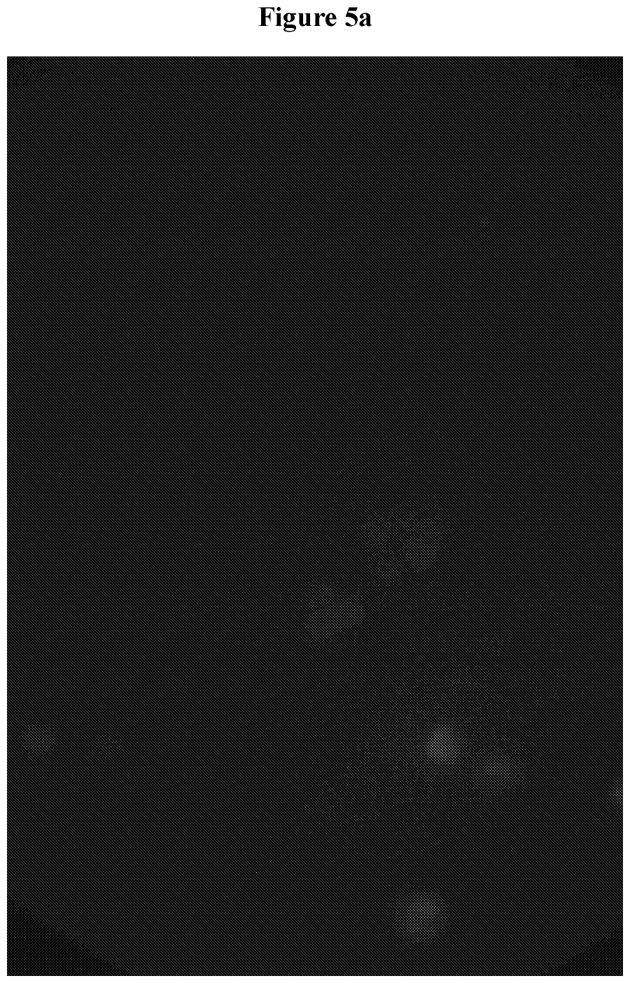

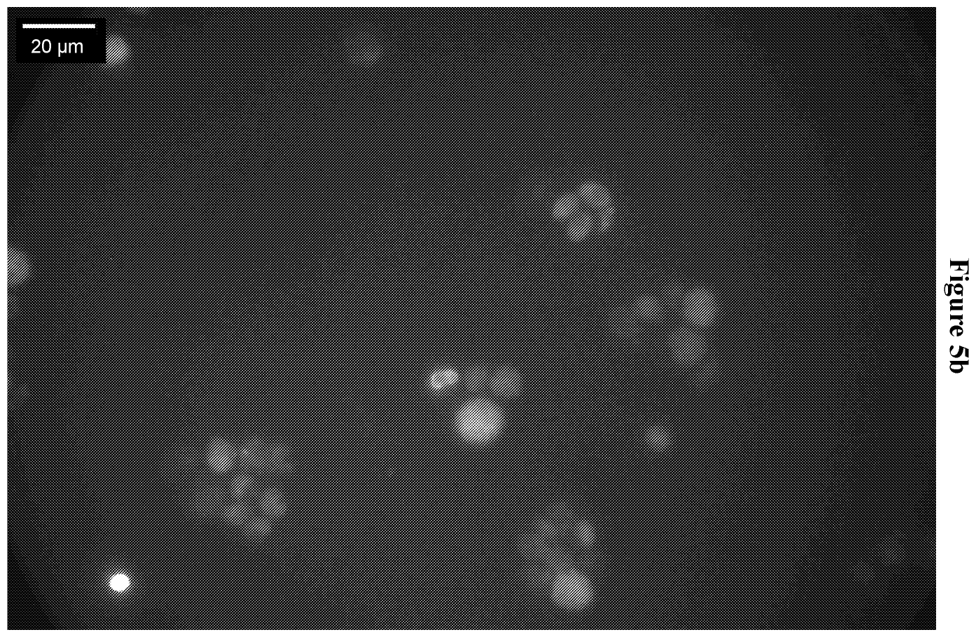

Fluorescence microscopy demonstrated that the exogenous fatty acid transporters locate to the cytoplasmic membrane of an engineered single-celled photosynthetic organism comprising an exogenous nucleic acid sequence of the invention. FIG. 5a shows C. reinhardtii transformed with a nucleic acid encoding an antibiotic selection marker only (mock control). Only background fluorescence is visible in FIG. 5a. FIG. 5b shows C. reinhardtii transformed with a nucleic acid encoding an ABCG15-clover fusion protein. Clusters of fluorescent cells are clearly visible in FIG. 5b, with the fluorescent staining concentrated in the cytoplasmic membrane.

The experimental results described in this example demonstrate that a single-celled photosynthetic organism engineered to comprise an exogenous nucleic acid sequence comprising a coding sequence for a fatty acid transporter operationally linked to a promoter sequence expresses the fatty acid transporter and that the fatty acid transporter is localized in the cytoplasmic membrane of the organism upon expression.

### Example 6. The Fatty Acid Transporters Effectively Transport Fatty Acids Across the Cytoplasmic Membrane from Inside the Cell

This example illustrates that an engineered single-celled photosynthetic organism comprising an exogenous nucleic acid sequence comprising a coding sequence for a fatty acid transporter operationally linked to a promoter sequence is capable of transporting across the cytoplasmic membrane from inside the cell into the surrounding culture medium.

To demonstrate that the fatty acid transporters function by secreting fatty acids out of the cells, C. reinhardtii cells were transformed with one of the nucleic acids described in Example 3, which comprise coding sequences for a fatty acid transporter operationally linked to a promoter sequence. No antibiotic was used during the assays.

The cells were artificially fattened to increase the intracellular fatty acid concentration (Bai et al. Long-chain acyl-CoA synthetases activate fatty acids for lipid synthesis, remodeling and energy production in Chlamydomonas, New Phytologist (2022) 233: 823-837). Briefly, the transformed cells were grown on TAP medium to a cell density of 2×10⁷, and then displaced into the nitrogen-depleted TAP medium with regular or triple acetate (TAP-N/T5AP-N). Cells were stained with the lipophilic fluorescent dye BODIPY: 30 μL of late log phase cells were gently mixed with 0.3 μL BODIPY 495/505 (100× dilution), concentration 1 mg/mL in DMSO. Cells were shielded from light and incubated for 5 minutes before placing 5 μL onto regular slides, sealed with #1.5 coverslips and nail polish.

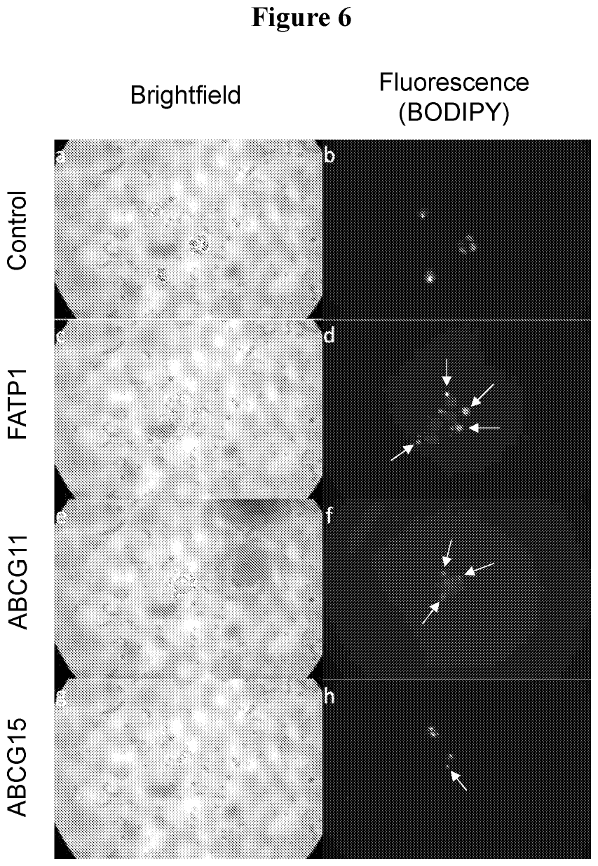

BODIPY is a stain for oil and other nonpolar lipids. After staining with BODIPY, the cells were subjected to fluorescence microscopy directly. Representative images are shown in FIG. 6. Panels a, c, e and g of FIG. 6 are brightfield images. Panels b, d, f and h of FIG. 6 are fluorescence images of the same field of view. Panels a and b show C. reinhardtii transformed with a nucleic acid encoding an antibiotic selection marker only (mock control). Panels c and d show C. reinhardtii transformed with a nucleic acid encoding the fatty acid transporter FATP1. Panels e and f show C. reinhardtii transformed with a nucleic acid encoding the fatty acid transporter ABCG11. Panels g and h show C. reinhardtii transformed with a nucleic acid encoding the fatty acid transporter ABCG15.

After 72 hours of nitrogen starvation, the mock transformed cells were much fattened as demonstrated by the clear lipid bodies taking up a significant fraction of the volume inside the cells (see panels b, d, f and h of FIG. 6). No extracellular oil depots were visible in control cells. The cells transformed with the fatty acid transporters showed striking differences as displayed in the representative images in FIG. 6. Extracellular oil globules were observed in near perfect spherical shape, or deposited on the surface of the cells (see arrows in panels d, f and h of FIG. 6).

The experimental results described in this example demonstrate that single-celled photosynthetic organisms engineered to comprise an exogenous nucleic acid sequence comprising a coding sequence for a fatty acid transporter operationally linked to a promoter sequence expresses are capable of transporting fatty acids across the cytoplasmic membrane from inside the cell, as a result of expressing the fatty acid transporter.

### Example 7. Fatty Acid Transporters Preferentially Transport Monounsaturated Fatty Acids

This example illustrates that an engineered single-cell photosynthetic organism comprising an exogenous nucleic acid sequence comprising a coding sequence for a fatty acid transporter operationally linked to a promoter sequence is capable of preferentially transporting monounsaturated long chain free fatty acids (C16:1 and C18:1) across the cytoplasmic membrane from inside the cell into the surrounding culture medium.

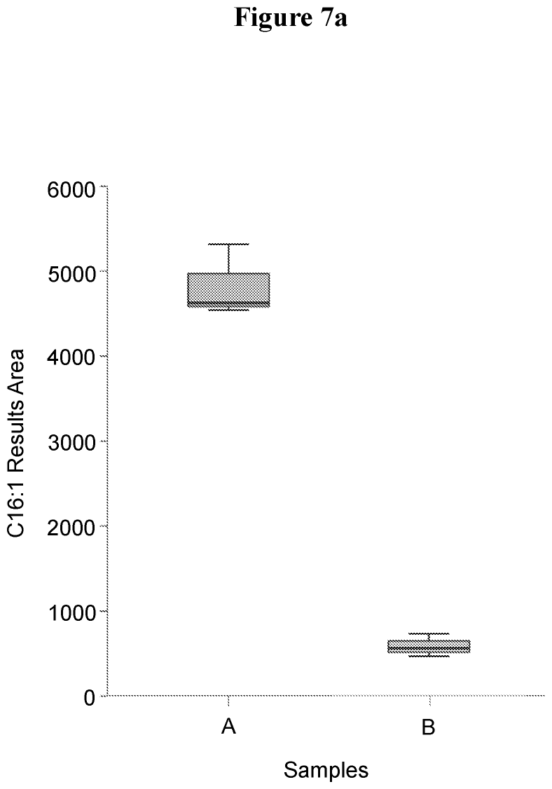

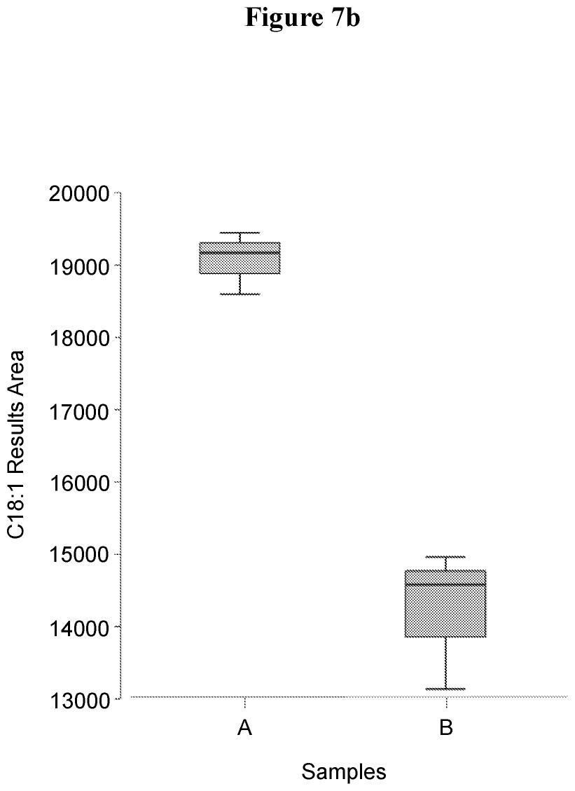

C. reinhardtii cells were transformed with SLC27A1 (encoding the Homo sapiens flippase FATP1) as described in Example 3, or the antibiotic cassette alone. A SLC27A1-expressing clone (sample A in FIGS. 7a and 7b) was selected to evaluate the fatty acids secreted into the cell medium and compared to a SLC27A1-non-expressing clone (sample B in FIGS. 7a and 7b). Clones were grown under selection to a cell density of 2×10⁵ and resuspended in a high-carbon low-nitrogen medium in the absence of antibiotics. Samples of cell-free medium were collected 5 days post-treatment, diluted with isopropanol and analysed by liquid chromatography-mass spectrometry (LC-MS).

The cell-free medium samples were evaluated for the presence of saturated (C14:0, C16:0 and C18:0), monounsaturated (C16:1 and C18:1) and polyunsaturated (C18:2) free fatty acids. Compared to medium obtained from a SLC27A1-non-expressing clone, medium from a SLC27A1-expressing clone contained significantly higher monounsaturated long chain free fatty acids, specifically about 5-fold more palmitoleic acid (C16:1) and about 1.5-fold more oleic acid (C18:1), respectively (see FIGS. 7a and 7b). No statistical differences were observed for the tested saturated or polyunsaturated free fatty acids.

The results described in this example demonstrate that a single-celled photosynthetic organism can be engineered to be capable of preferentially secreting monounsaturated fatty acids with a chain length of C16 to C18 from inside the cell.

### Example 8. Expression of an Exogenous Fatty Acid ABC Transporter in a Single-Celled Photosynthetic Organisms Suitable for Biofuel Production

This example demonstrates successful transformation of the single-celled photosynthetic organism Chlorella sorokiniana with an exogenous nucleic acid comprising a coding sequence for an exemplary fatty acid ABC transporter (ABCG11).

C. sorokiniana is one of the most promising microalgae strains that emerged from the National Alliance for Advanced Biofuels and Bioproducts (NAABB) consortium project. C. sorokiniana cells (strain number: SAG 211-32) were transformed using a PEG-based method of generating protoplasts. Cells were grown to mid-log phase, concentrated and resuspended in pectinase and cellulase to remove cell walls enzymatically. Cells were washed and then incubated with PEG 8000 and linear plasmids containing expression cassettes comprising the fatty acid transporter gene ABCG11 and/or an antibiotic resistance gene generated in Example 3.

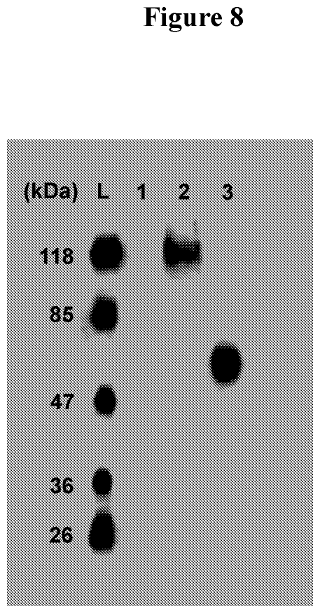

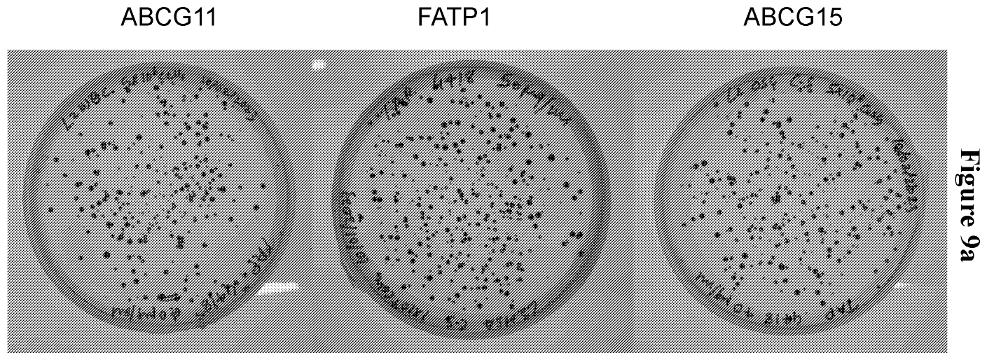

Cells were cultured in TAP medium prior to cell lysis in 2×LDS buffer at 95° C. Gel electrophoresis was performed using a Thermo Bis-Tris polyacrylamide gel followed by protein transfer onto a PVDF membrane using the Invitrogen iBlot2. The membrane was blocked with 7.5% skimmed milk for 20 minutes prior to incubation with anti-His tag antibody (66005-1-Ig). Protein expression was detected using a goat anti-mouse secondary antibody that could be visualized with Li-Cor C-Digit. ABCG11 transgene expression was visualized by a protein band at ˜118 kDa, corresponding to ABCG11+6His-tag (see FIG. 8).

This example demonstrates the successful transformation of the single-celled photosynthetic organism C. sorokiniana cells with an exogenous nucleic acid comprising a coding sequence for a fatty acid ABC transporter.

### Example 9. Expression of Various Exogenous Fatty Acid Transporters

This example demonstrates successful transformation of the single-celled photosynthetic organism Chlorella sorokiniana with an exogenous nucleic acid encoding either a plant-derived fatty acid ABC transporter (ABCG11 or ABCG15) or a mammalian-derived flippase (FATP1).

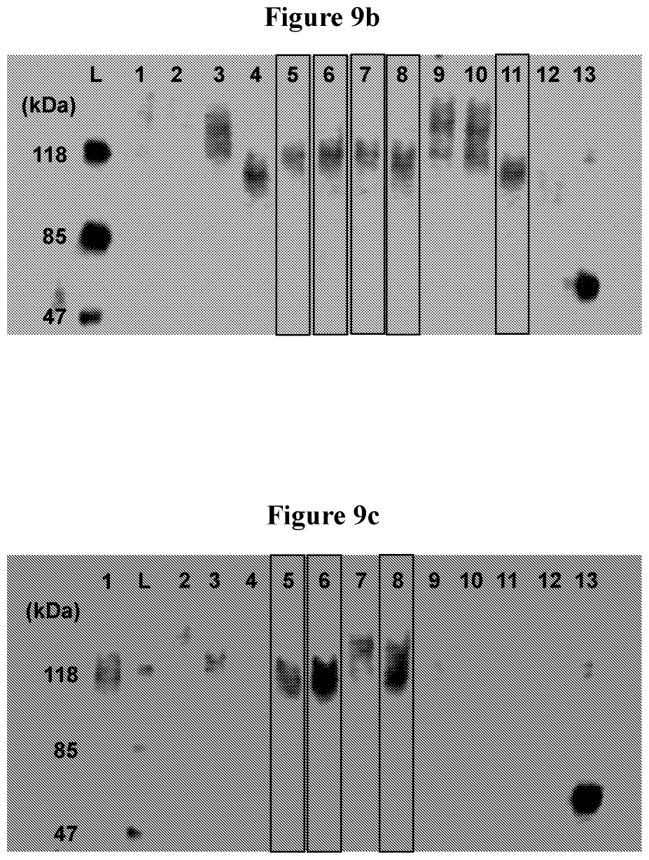

C. sorokiniana cells (strain number: SAG 211-32) were transformed via a biolistic transformation process. Expression plasmids were precipitated onto solid-phase gold particles which were projected into the microalgae using a PDS-1000 biolistic particle delivery system (BioRad) according to the manufacturer's instructions. Following particle bombardment, the cells were resuspended and plated onto agar-TAP plates in the presence of G418 (see FIG. 9a) to select transformants.

Transformed cells were identified after a 5-day selection. Clones were isolated and grown in liquid TAP medium. As described in Example 8, Western blot was used to determine expression of FATP1, ABCG15 and ABCG11 transgenes (FIGS. 9b and 9c). Not all clones successfully expressed the respective transgene. Samples in which successful transgene expression was detected are indicated in FIGS. 9b and 9c by black rectangles.

This example demonstrates the successful transformation of the single-celled photosynthetic organism C. sorokiniana cells with an exogenous nucleic acid encoding either a plant-derived fatty acid ABC transporter (ABCG11 or ABCG15) or a mammalian-derived flippase (FATP1).

### Example 10. Engineered Single-Celled Photosynthetic Cells Expressing Exogenous Fatty Acid Transporters Secrete Fatty Acids

This example demonstrates that engineered single-cell photosynthetic organisms expressing an exogenous fatty acid transporter secrete fatty acids into the culture medium.

C. sorokiniana cells were transformed with one of the three fatty acid transporter-encoding nucleic acids as described in Example 9. For each transgene, a set of four representative transformants that successfully expressed the transgene was selected to assess fatty acid secretion into a liquid culture medium. Prior to the secretion assay, transgene expression was confirmed by Western blot as described in Example 9. The results are shown in FIG. 10a. Bands corresponding in their molecular weight to FATP1, ABCG11 and ABCG15, respectively, were detected using an anti-His tag antibody.

Liquid cultures (in TAP medium) were prepared with one of the transformants of each set. A culture with a corresponding wild-type cell was incubated under the same conditions. Each culture was subjected to nitrogen starvation for 2 days, as described in Example 7. Cell-free medium samples were collected, and their free fatty acid content was determined using a free fatty acid quantification kit (Abcam). The free fatty acid content was normalized by cell density.

Unlike the wild-type control, all fatty acid transporter-expressing transformants secreted readily detectable amounts of fatty acids in the growth medium (see FIG. 10b).

This example demonstrates that an engineered single-cell photosynthetic organism comprising an exogenous nucleic acid for the expression of either a plant-derived fatty acid ABC transporter (ABCG11 or ABCG15) or a mammalian-derived flippase (FATP1) readily secretes fatty acids into the culture medium.

1. An engineered single-celled photosynthetic organism comprising an exogenous nucleic acid sequence comprising a coding sequence for a fatty acid transporter operationally linked to a promoter sequence.

2. The engineered organism of claim 1, wherein the fatty acid transporter is localized in the plasma membrane of the organism upon expression.

3. The engineered organism of claim 1, wherein the fatty acid transporter is capable of transporting fatty acids with a carbon chain length of C14 to C22 across the plasma membrane of the organism from inside the cell, optionally wherein the fatty acids are monounsaturated.

4. The engineered organism of claim 1, wherein the coding sequence for the fatty acid transporter is derived from a genome of a plant cell or a mammalian cell.

5. The engineered organism of claim 4, wherein the coding sequence for the fatty acid transporter is derived from the genome of a plant cell.

6. The engineered organism of claim 5, wherein the fatty acid transporter is an ABC transporter.

7. The engineered organism of claim 6, wherein the ABC transporter is (a) an Arabidopsis thaliana ABCG11 protein or a functional homolog thereof, or (b) an Oryza sativa ABCG15 protein or a functional homolog thereof.

8. The engineered organism of claim 4, wherein the coding sequence for the fatty acid transporter is derived from the genome of a mammalian cell.

9. The engineered organism of claim 8, wherein the fatty acid transporter is an ABC transporter or flippase.

10. The engineered organism of claim 9, wherein the flippase is a Homo sapiens FATP 1 protein or a functional homolog thereof.

11. The engineered organism of claim 1, wherein the engineered organism is an algae, optionally wherein the algae is an oleaginous algae.

12. The engineered organism of claim 11, wherein the engineered organism is an algae selected from the group consisting of Chlorophyta, Phaeophyta, Rhodophyta, Xanthophyta, Chrysophyta, Bacillariophyta, Cryptophyta, Dinophyta, Euglenophyta, Cyanophyta and Myxophyta.

13. A culture comprising the engineered organism of claim 1.

14. A method for producing fatty acids comprising culturing the engineered organism of claim 1 in a medium suitable for growing the engineered organism.

15. A method of producing a biofuel, comprising: a) providing a fatty acid obtained by the method of claim 14; and b) processing the fatty acid by transesterification, decarboxylation or hydrocracking.


SEQUENCE LISTING (25 sequences)

SEQ ID NO: 1 - protein, 703 aa, Arabidopsis thaliana
MEIEASRQQTTVPVSVGGGNFPVGGLSPLSEAIWREKAPTEFVGDVSARLTWQDLTVMVTMGDGETQNVLEGLTGYAEPGSLTALMGPSGSGKSTMLDALASRLAANAFLSGTVLLNGRKTKLSFGTAAYVTQDDNLIGTLTVRETIWYSARVRLPDKMLRSEKRALVERTIIEMGLQDCADTVIGNWHLRGISGGEKRRVSIALEILMRPRLLFLDEPTSGLDSASAFFVTQTLRALSRDGRTVIASIHQPSSEVFELFDRLYLLSGGKTVYFGQASDAYEFFAQAGFPCPALRNPSDHFLRCINSDFDKVRATLKGSMKLRFEASDDPLEKITTAEAIRLLVDYYHTSDYYYTAKAKVEEISQFKGTILDSGGSQASFLLQTYTLTKRSFINMSRDFGYYWLRLLIYILVTVCIGTIYLNVGTSYSAILARGSCASFVFGFVTFMSIGGFPSFVEDMKVFQRERLNGHYGVAAFVIANTLSATPFLIMITFISGTICYFMVGLHPGFTHYLFFVLCLYASVTVVESLMMAIASIVPNFLMGIIIGAGIQGIFMLVSGFFRLPNDIPKPFWRYPMSYISFHFWALQGQYQNDLRGLTFDSQGSAFKIPGEYVLENVFQIDLHRSKWINLSVILSMIIIYRIIFFIMIKTNEDVTPWVRGYIARRRMKQKNGTQNTTVAPDGLTQSPSLRNYIATRTDGARRW

SEQ ID NO: 2 - protein, 694 aa, Oryza sativa
MCLLEEMMEISSNEEMMEMAIVEQLPPSSHHLNGGSVEVDMEEDHVWPTKDGPLPIFLKFENVEYKVKLTPKNPLTAARVAFASHKSTEDQGSCKHILKGVGGSVDPGEILALMGPSGSGKTTLLKILGGRLSGGVRGQITYNDTPYSPCLKRRIGFVTQDDVLFPQLTVEETLVFAAFLRLPARMSKQQKRDRVDAIITELNLERCRHTKIGGAFVRGVSGGERKRTSIGYEILVDPSLLLLDEPTSGLDSTSAAKLLVVLRRLARSAARRTVITTIHQPSSRMFHMFDKLLLVAEGHAIYHGGARGCMRHFAALGFSPGIAMNPAEFLLDLATGNLDGISSPASLLLPSAAAASPDSPEFRSHVIKYLQARHRAAGEEEAAAAAAREGGGGGGAGRDEAAKQLRMAVRMRKDRRGGIGWLEQFTVLSRRTFRERAADYLDKMRLAQSVGVALLLGLLWWKSQTSNEAQLRDQVGLIFYICIFWTSSSLFGSVYVFPFEKLYLVKERKADMYRLSAYYASSTVCDAVPHVVYPVLFTAILYFMADLRRTVPCFCLTLLATLLIVLTSQGTGELLGAAILSVKRAGVMASLVLMLFLLTGGYYVQHIPKFIRWLKYVSFMHYGFNLLLKAQYHGHLTYNCGSRGGCQRLQSSPSFGTVDLDGGMREVWILLAMAVAYRLLAYLCLRKRISLMPL

SEQ ID NO: 3 - protein, 646 aa, Homo sapiens
MRAPGAGAASVVSLALLWLLGLPWTWSAAAALGVYVGSGGWRFLRIVCKTARRDLFGLSVLIRVRLELRRHQRAGHTIPRIFQAVVQRQPERLALVDAGTGECWTFAQLDAYSNAVANLFRQLGFAPGDVVAIFLEGRPEFVGLWLGLAKAGMEAALLNVNLRREPLAFCLGTSGAKALIFGGEMVAAVAEVSGHLGKSLIKFCSGDLGPEGILPDTHLLDPLLKEASTAPLAQIPSKGMDDRLFYIYTSGTTGLPKAAIVVHSRYYRMAAFGHHAYRMQAADVLYDCLPLYHSAGNIIGVGQCLIYGLTVVLRKKFSASRFWDDCIKYNCTVVQYIGEICRYLLKQPVREAERRHRVRLAVGNGLRPAIWEEFTERFGVRQIGEFYGATECNCSIANMDGKVGSCGFNSRILPHVYPIRLVKVNEDTMELLRDAQGLCIPCQAGEPGLLVGQINQQDPLRRFDGYVSESATSKKIAHSVFSKGDSAYLSGDVLVMDELGYMYFRDRSGDTFRWRGENVSTTEVEGVLSRLLGQTDVAVYGVAVPGVEGKAGMAAVADPHSLLDPNAIYQELQKVLAPYARPIFLRLLPQVDTTGTFKIQKTRLQREGFDPRQTSDRLFFLDLKQGHYLPLNEAVYTRICSGAFAL

SEQ ID NO: 4 - DNA, 2109 bp, synthetic construct
  source 1..2109: mol_type = other DNA
atggagatcgaggccagccgccagcagaccaccgtgcccgtgagcgtgggcggcggcaacttccccgtgggcggcctgagccccctgagcgaggctatctggcgcgagaaggctcccaccgagttcgtgggcgatgtgagcgctcgcctgacctggcaggatctgaccgtgatggtgaccatgggcgatggtgagactcagaacgtgctggagggtctgactggctacgccgagcccggcagcctgaccgctctgatgggccctagcggcagcggtaagagcaccatgctggatgctctggctagccgcctggccgctaacgctttcctgagcggtaccgtgctgctgaacggtcgcaagaccaagctgagcttcggtaccgctgcctacgtgacccaggacgataacctgatcggcaccctgaccgtgcgcgagactatctggtacagcgctcgcgtgcgcctgcccgacaagatgctgcgcagcgagaagcgcgccctggtggagcgcaccatcattgagatgggcctgcaggactgcgctgacaccgtgattggcaactggcacctgcgcggcatcagcggcggcgagaagcgccgcgtgagcatcgctctggagatcctgatgcgcccccgcctgctgtttctggacgagcccaccagcggcctggacagcgccagcgctttctttgtgacccagaccctgcgcgctctgagccgcgatggccgcactgtgatcgctagcattcaccagcccagctctgaggtgtttgagctgtttgaccgcctgtacctgctgagcggtggcaagaccgtgtacttcggccaggccagcgacgcttacgagttcttcgcccaggccggcttcccttgccccgctctgcgcaaccctagcgaccacttcctgcgctgcatcaacagcgatttcgacaaggtgcgcgccaccctgaagggcagcatgaagctgcgcttcgaggctagcgacgatcccctggagaagatcaccaccgccgaggccatccgcctgctggtggactactaccacaccagcgactactactacaccgccaaggctaaggtggaggagatcagccagttcaagggcaccatcctggacagcggcggcagccaggccagcttcctgctgcagacctacaccctgaccaagcgcagcttcatcaacatgagccgcgacttcggctactactggctgcgcctgctgatctacatcctggtgaccgtgtgcatcggcaccatctacctgaacgtgggcaccagctacagcgctatcctggcccgcggcagctgcgccagcttcgtgttcggcttcgtgaccttcatgagcatcggcggcttccccagcttcgtggaggacatgaaggtgttccagcgcgagcgcctgaacggccactacggcgtggctgccttcgtgatcgccaacaccctgagcgctacccccttcctgatcatgatcaccttcatcagcggcaccatctgctacttcatggtgggcctgcaccccggcttcacccactacctgttcttcgtgctgtgcctgtacgccagcgtgaccgtggtggagagcctgatgatggctatcgccagcatcgtgcccaacttcctgatgggcatcatcatcggcgccggcatccagggcatcttcatgctggtgagcggcttcttccgcctgcccaacgacatccccaagcccttctggcgctaccccatgagctacatcagcttccacttctgggccctgcagggccagtaccagaacgacctgcgcggcctgaccttcgacagccagggcagcgctttcaagatccccggcgagtacgtgctggagaacgtgttccagatcgacctgcaccgcagcaagtggatcaacctgagcgtgatcctgagcatgatcatcatttaccgcatcattttctttatcatgattaagaccaacgaggacgtgaccccttgggtgcgcggctacatcgctcgccgccgcatgaagcagaagaacggcacccagaacactactgtggctcctgacggcctgacccagagccctagcctgcgcaactacattgctacccgcactgacggtgctcgccgctgg

SEQ ID NO: 5 - DNA, 2082 bp, synthetic construct
  source 1..2082: mol_type = other DNA
atgtgcctgctggaggagatgatggagatcagcagcaacgaggagatgatggagatggccatcgtggagcagctgccccccagcagccaccacctgaacggcggtagcgtggaggtggacatggaggaggatcacgtgtggcccaccaaggacggccctctgcccattttcctgaagttcgagaacgtggagtacaaggtgaagctgacccctaagaaccccctgactgccgctcgcgtggccttcgctagccacaagagcaccgaggaccagggcagctgcaagcacatcctgaagggcgtgggcggtagcgtggatcctggcgagatcctggccctgatgggccccagcggcagcggtaagaccactctgctgaagatcctgggcggtcgcctgagcggcggtgtgcgcggccagatcacctacaacgacaccccttacagcccctgcctgaagcgccgcattggcttcgtgactcaggatgacgtgctgttccctcagctgaccgtggaggaaaccctggtgttcgccgctttcctgcgcctgcccgcccgcatgagcaagcagcagaagcgcgaccgcgtggatgctatcattactgagctgaacctggagcgctgccgccacaccaagatcggcggtgccttcgtgcgcggcgtgagcggcggtgagcgcaagcgcaccagcattggctacgagatcctggtggatcctagcctgctgctgctggacgagcccactagcggtctggacagcaccagcgccgctaagctgctggtggtgctgcgccgcctggcccgcagcgccgctcgccgcaccgtgatcaccactattcaccagcctagcagccgcatgttccacatgttcgacaagctgctgctggtggccgagggccacgccatctaccacggcggtgcccgcggctgcatgcgccacttcgccgctctgggcttcagccccggtattgccatgaaccctgccgagttcctgctggacctggccaccggcaacctggacggtatcagcagccccgctagcctgctgcttcctagcgccgccgctgctagccccgattctcctgagttccgcagccacgtgattaagtacctgcaggctcgtcaccgcgctgctggcgaggaggaagccgctgccgccgccgctcgtgagggtggtggtggtggtggtgccggccgcgacgaggccgctaagcagctgcgcatggccgtgcgcatgcgcaaggatcgccgcggcggtattggctggctggagcagttcaccgtgctgagccgccgcactttccgcgagcgcgccgctgactacctggataagatgcgcctggcccagagcgtgggcgtggccctgctgctgggcctgctgtggtggaagagccagaccagcaacgaggctcagctgcgcgaccaggtgggtctgatcttctacatttgcatcttctggaccagcagcagcctgttcggcagcgtgtacgtgttccctttcgagaagctgtacctggtgaaggagcgcaaggccgacatgtaccgcctgagcgcctactacgctagcagcactgtgtgcgatgccgtgccccacgtggtgtaccctgtgctgttcaccgccattctgtacttcatggctgacctgcgccgcaccgtgccctgcttctgcctgactctgctggccaccctgctgatcgtgctgaccagccagggcactggtgagctgctgggcgccgctattctgagcgtgaagcgcgccggcgtgatggctagcctggtgctgatgctgttcctgctgaccggcggttactacgtgcagcacatccccaagttcattcgctggctgaagtacgtgagcttcatgcactacggcttcaacctgctgctgaaggcccagtaccacggccacctgacctacaactgcggcagccgcggcggttgccagcgcctgcagagcagccctagcttcggtaccgtggacctggatggcggtatgcgcgaggtgtggatcctgctggctatggccgtggcctaccgcctgctggcttacctgtgcctgcgcaagcgcattagcctgatgccccta

SEQ ID NO: 6 - DNA, 1938 bp, synthetic construct
  source 1..1938: mol_type = other DNA
atgcgcgcccccggcgccggcgccgccagcgtggtgagcctggccctgctgtggctgctgggcctgccctggacctggagcgccgccgccgccctgggcgtgtacgtgggtagcggcggttggcgcttcctgcgcatcgtgtgcaagaccgcccgccgtgacctgtttggcctgagcgtgctgatccgcgtgcgcctggagctgcgccgccaccagcgtgctggccacaccatcccccgcatcttccaagccgtggtgcagcgccagcccgagcgcctggccctggtggacgctggcaccggtgagtgctggaccttcgctcagctggacgcctacagcaacgccgtggccaacctgttccgtcagctgggcttcgcccccggtgacgtggtggctatcttcctggagggtcgccccgagtttgtgggcctgtggctgggtctggctaaggccggcatggaggccgctctgctgaacgtgaacctgcgccgtgagcccctggccttctgcctgggcaccagcggtgctaaggccctgatcttcggcggtgagatggtggccgctgtggccgaggtgagcggccacctgggtaagagcctgatcaagttctgcagcggcgacctgggtcctgagggcattctgcccgacacccacctgcttgaccccctgcttaaggaggccagcaccgctcccctggcccagatccccagcaagggtatggacgatcgcctgttctacatctacaccagcggcaccaccggtctgcccaaggccgctattgtggtgcacagccgctactatcgcatggccgctttcggccaccacgcctaccgcatgcaggccgctgacgtgctgtacgattgcctgcctctgtaccacagcgccggcaacatcattggcgtgggtcagtgcctgatctacggcctgaccgtggtgctgcgtaagaagttcagcgctagccgcttttgggacgattgcatcaagtacaactgcaccgtggtgcagtacattggcgagatttgccgctacctgctgaagcagcccgtgcgcgaggccgagcgccgccaccgtgtgcgcctggccgtgggcaacggcctgcgccccgccatttgggaggagttcaccgagcgcttcggcgtgcgccagatcggtgagttctacggcgccaccgagtgcaactgcagcattgccaacatggacggtaaggtgggcagctgcggtttcaacagccgcatcctgccccacgtgtaccctatccgtcttgtgaaggtgaacgaggataccatggagctgctgcgcgacgcccagggtctgtgcatcccttgccaggccggcgagcccggtctgcttgtgggtcagatcaaccagcaagaccccctgcgccgcttcgatggttacgtgagcgagagcgccaccagcaagaaaattgcccacagcgtgttcagcaagggtgacagcgcctacctgagcggcgatgtgctggtgatggatgagctgggctacatgtacttccgcgaccgcagcggtgacacctttcgctggcgcggtgagaacgtgagcaccactgaggtggagggcgtgctgagccgcctgctgggccagaccgacgtggctgtgtacggcgtggccgtgcccggtgtggagggcaaggccggcatggccgctgtggctgacccccacagcctgctggatcccaacgctatctaccaggagctgcagaaggtgctggccccctacgctcgccctattttcctgcgcctgctgccccaggtggacaccaccggtaccttcaagatccaaaaaacccgcctgcagcgcgagggcttcgacccccgccagaccagcgatcgcctgttctttctggatctgaagcagggccactacctgcccctgaacgaggctgtgtacacccgtatttgcagcggcgccttcgctctg

SEQ ID NO: 7 - DNA, 734 bp, Cauliflower mosaic virus
  source 1..734: mol_type = genomic DNA
actctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctga

SEQ ID NO: 8 - DNA, 351 bp, Cauliflower mosaic virus
  source 1..351: mol_type = genomic DNA
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctga

SEQ ID NO: 9 - DNA, 198 bp, Chlamydomonas reinhardtii
  source 1..198: mol_type = genomic DNA
ctggcactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagc

SEQ ID NO: 10 - DNA, 477 bp, synthetic construct
  source 1..477: mol_type = other DNA
ctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaatacttga

SEQ ID NO: 11 - DNA, 640 bp, synthetic construct
  source 1..640: mol_type = other DNA
cgcgacggttcgagaaccgacttgagggcgccaaacgagcccgagccgccgttgcgccaggcgaaaccagaaccgtagattaatgcacttgagctattcattggagcgatctgccggggacagcgggtctggcgtgcgcgcgattggagatcgcaaattacatatgtctgcgtgacggcggggagctcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcgatcccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaag

SEQ ID NO: 12 - DNA, 485 bp, Chlamydomonas reinhardtii
  source 1..485: mol_type = genomic DNA
gtaggtcaggaccagagcctacaacattgtaaggccatgtatcctccagtgccatttgcgacgttacacgacgagacaatctgcgctgtgttcaagcacacacatccgggactcgtgtgatgtcttaacatctgcaaggaaacataaggtggccggcgtcacgcaagatgacgcgacggactcgaatgcttgcttcgtccagcgacaaataaggacaggcagggcgcctgcattgcgtgcgcttagaggcgaggcgctcaagacatttcggcagcaataattggtattgaggcacattctgcaccaggatgccaagaggtgaacgttgctgccttaatgtatatctgcacagctggccggttactgcgaaccgggtgccttttggagtcgctcctaaaagacgtcaccggcgcaacgtcgtcgtgcggccgtacaggtatgcaaaattggcccggttgcgcgcaggccgattatttaagtgacat

SEQ ID NO: 13 - DNA, 251 bp, Chlamydomonas reinhardtii
  source 1..251: mol_type = genomic DNA
cccgccagcccccgctcctctgctgcctctgatgcctcatgccaaaagtcctgacgcggcgccctcacatccccgtccgggtaatctatgagtttcccttatcgagcatgtacgcgatagtggacggggctcagggtggggggtgggtgggtgggaggggcgttccttcagacaccctggaggggtggctagaaaagcggccgcgcgccagaaatgtctcgctgccctgtgcaataagcaccggctatatt

SEQ ID NO: 14 - DNA, 776 bp, Chlamydomonas reinhardtii
  source 1..776: mol_type = genomic DNA
gatcccacacacctgcccgtctgcctgacaggaagtgaacgcatgtcgagggaggcctcaccaatcgtcacacgagccctcgtcagaaacacgtctccgccacgctctccctctcacggccgaccccgcagcccttttgccctttcctaggccaccgacaggacccaggcgctctcagcatgcctcaacaacccgtactcgtgccagcggtgcccttgtgctggtgatcgcttggaagcgcatgcgaacacgaaggggcggagcaggcggcctggctgttcgaagggctcgccgccagttcgggtgcctttctccacgcgcgcctccacacctaccgatgcgtgaaggcaggcaaatgctcatgtttgcccgaactcggagtccttaaaaagccgcttcttgtcgtcgttccgagacatgttagcagatcgcagtgccacctttcctgacgcgctcggccccatattcggacgcaattgtcatttgtagcacaattggagcaaatctggcgaggcagtaggcttttaagttgcaaggcgagagagcaaagtgggacgcggcgtgattattggtatttacgcgacggcccggcgcgttagcggcccttcccccaggccagggacgattatgtatcaatattgttgcgttcgggcactcgtgcgagggctcctgcgggctggggagggggatctgggaattggaggtacgaccgagatggcttgctcggggggaggtttcctcgccgagcaagccagggttaggtgttgcgctcttga

SEQ ID NO: 15 - DNA, 583 bp, Chlamydomonas reinhardtii
  source 1..583: mol_type = genomic DNA
aaacagcacagaagggaaaggccgggtgtcggaaaaacgcgctccggaacggccttcctgaagctcgcgcacattcccaccctttcaccccgcaataaccgctcgccgctcgttgctaatgctgatagttgttgcgcacttgcgcccacgcgccggtgggacagccccggcaagccagatttctcaccggcgtcaccatccgcagccgcagcggcagcgcctgccaccagcagcgccgcaagtgccagcgccggcagaagcagtcccgcgcattggtgggttggcatggttgttttggcgggtgcgcgcaaagagctctggtagactacggcgagtcaagcggcgcggtcgcacggagaaaggtcgggccctggaaaagcgtttcggaagggggcgtcggtgcagctaggacggggaatgacgagaggtggctgctcgcgggggagcacagctgcaggccggccgcggacttgcgcggagctgcgccgggaggccgaggccgaccccccgcgccccatgcaaaaggcgaaggcccctgcgtaaattcgcgcacgccaaacttatagggtttgaactataagca

SEQ ID NO: 16 - DNA, 287 bp, Chlorella vulgaris
  source 1..287: mol_type = genomic DNA
tgatgcaaacaattctattgatcatcatgctgcatacttcaatgccttgatgcacaagctctcacaacaagcatgtcctcccgcatgcacatcgtttccccctcacaaaatctgtctctggtttggtggtcagctggcgtgttttgcgggcgttccctctttcagacaccacaccagagcgtcgcgcgcaaaacttacaaaattggatgacgcgtcctcgttcgccgtctgctcctctttcttaaagcctccaagccacccagcttctttctaaatctgcaaccatg

SEQ ID NO: 17 - DNA, 460 bp, Dunaliella tertiolecta
  source 1..460: mol_type = genomic DNA
cttttgtctgtacagacctggtttgctcaagcctagcagcgcattggaaatccactggaaatcccaaatgtggcgcacaggaatatgctcaggactttttcttgctcgtgcgcacctgatgtcaacccttctcaaggtttggcgcacctaggaagcaccctcactgaggtgcttccttgagagcccatgcaatcaaaacacctggtccactgacacccatttactgtatcaaggcgattgcttgataaatgaggctgtgcaggattggaggcggtgcgttcatctgctgcttgcaggctcttcccagcacttgcattcttccttgcatcctccactcagctcacaagcccacccctgaaagcactcacgcagcacaacccgctgcattttaaacaacgctgtccaacagggcctcctcactctcacactacctcaacaaacaaggggaagttggttacta

SEQ ID NO: 18 - DNA, 250 bp, Nannochloropsis gaditana
  source 1..250: mol_type = genomic DNA
acttgtgcgtggttacttgccgacagaatggcgtgtccttattcggcctggtgaccgtcccggtcgtagaacgttctgtctcccacccttccttccaaggcctgaagtcgttttgtgaccgtaattttcaatttgcgtgcaaggcctacaagtgagggctttatctatgcgcatttgacatggtgctgcttatcaacgcttatcctgtccggccccatttccacgcaacgtcattttttgacagactaac

SEQ ID NO: 19 - DNA, 1319 bp, Nannochloropsis gaditana
  source 1..1319: mol_type = genomic DNA
ggcttctcacgtcgttctcatcgtctgctttctccccctttccttttcttccgtcgtttctcccacagaacaattacgggagatgctggatttctatgtggaggctgacgttttccgggtcagcgccgatcggacgatgattgaactcttcaacacggattgagagagcactgtacgcagtgcagagcgtggtaggacatagaatcgcgcagggaaataggttggaaggggagcaattcgcagaaggaaagctcagcgatcgagaaaaacatggagggtgaagataagctcgctctgaaagtaaatctgacaaatcaagcacagatgaggtcactgcgaagttgacctgaaagcgtgtttttcaaatcgtcgcccaggcgaagtgttattatttcttgcacatcttgaagatagtctatacgcaggatgaaaaggaggatcaactttgccatttctgcatacgtcataattccacgatgactagcaatgtggaatagatcttccttttgaatttctctgaatggttagcactttcctttgtttttttgtcctatgaaatttaaatggtaattgatacctccactaaggagtcaaatcaatagctttgatcacaagcagaagaaatgataagagcgcaaagcttggaataatctctttatatgtcccattgattaacatctatcctacaagggcgtgaacaaccacgatcagagtttaggagagcgatcaactccaagcactaaaagggcgtgaacaaccactataagaattgaagaggcgatcctccaagaacgctgcccgaccaggcattaattgccgcataatctgaccgtcgtctggatagcctggcagacgggaaaacttcatggaataatgacgccattacttgaagtttcgcggggctttttttcctggctcggaattatactttttacgccataactgcgtttccccgttctccaagggctccgccgtcggcaactttgtatggggaagcgcggtagattttgttttcaaggaaggaccaagggtgtgaggtggcttttgaaacactggtgccatgtatgggactattctgacggatctccgccgcgcaacttaactcccgcctcggattccgtgttaggcgtcgtaacgaacattaggaagacatcggatcttagatgagaaattatgcatcatgcgacttgtgtgaatgtgcatcaatcttttgcctgttgcattacgcatgctcacgcgacttcatgaatatgtcttcccttacatactcctcctgatcacgacaacacaacatttctcacatcactccagggtggtcg

SEQ ID NO: 20 - DNA, 693 bp, Nannochloropsis gaditana
  source 1..693: mol_type = genomic DNA
catcctgctgtatgattttggcacaacgacgcggttggagggtgagggagatggtggtgagacatcagaaatggagagaataacggtagagaaaaaaaggaacacacgcgcccaggatagaaccaggaccacattccaagtccctcaccagcaacaagagggacaccacgtcggtgaagaggtgcgtgagacagaggacaccagacaacgtgcacaaagcgtcggaaagggaggcgaccgcttcgctggaggggggccgtccacagcctgcgcccaaccacacacattcctcccgcgaggctcaagtgccatgccgccgagtgccatgctcctgcatccggggcaggtcgacgagccagcatggtttggtcgccgacaacgcagatttgatggagcttgggcatttctttaccggacacgtcgcttcttacatttttacattccaattcagaggtcacacccgcgatcgagaaagcagagagtcggacccccttcaagcaggaaaggcaagcaacatacccatcgttaagtctgatgatggctggtgcaaagccaaggcagccagtcgatatgttaatgatcgggtacgtttaaaggcgtgggcaaggttatttaacgctggtagcatcgtagagtttttaacgcaactgaagatttcatgtctgacttggagatacggtcggggcagcagct

SEQ ID NO: 21 - DNA, 422 bp, Phaeodactylum tricornutum
  source 1..422: mol_type = genomic DNA
catatgcggaagtgactgtaaacgagaagtgcacgaagccttttcttgtgacgtcacaaaccgaacagccctacgtggcgtgcgactttccggcactttggatatcgttgccctatatgtattgggtgtatacatgtcgtattccaactacgacagaaaacagtcagttacgggtattcgaacttccggcacctgcagcgaggagttttgtgtccgtcgcacatcctccgtgctttcggcacctacgtaggcggcaaacttcctgccttccgcccgccttgcggcattccggctccgggccagaattgcccgggtgttcacaattttgcctcctcacgaaaaaacgttctactttgtattttggtcgggtttcggatccttccagcaaccattctcattcaaagtcaccacttgtgcgaacg

SEQ ID NO: 22 - DNA, 253 bp, Agrobacterium tumefaciens
  source 1..253: mol_type = genomic DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 23 - DNA, 243 bp, Phaeodactylum tricornutum
  source 1..243: mol_type = genomic DNA
aaccttccttaaaaatttaattttcattagttgcagtcactccgctttggtttcacagtcaggaataacactagctcgtcttcaccatggatgccaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgag

SEQ ID NO: 24 - DNA, 1064 bp, synthetic construct
  source 1..1064: mol_type = other DNA
atgatcgagggcagggtgagcaagggcgaggagctgttcaccggcgtggtgcccatcctggtggagctggacggcgacgtgaacggccacaagttcagcgtgcgcggcgagggcgagggcgacgccaccaacggcaagctgaccctgaagttcatctgcaccaccggcaagctgcccgtgccctggcccaccctggtgaccaccttcggctacggcgtggcctgcttcagccgctaccccgaccacatgaagcagcacgacttcttcaagagcgccatgcccgagggctacgtgcaggagcgcaccatcagcttcaaggacgacggcacctacaagacccgcgccgaggtgaagttcgagggcgacaccctggtgaaccgcatcgagctgaagggcatcgacttcaaggaggacggtgagcttgcggggttgcgagcaacactccagcaacgaacagtgcccaagtcaggaatctgcagtcagcctgggctttcggcggctttttcttgggcaaacagcttgcactcatgccagcgcggcttgtccagcctcacttgagctttccagctgctaccagccgggctatacgacagcgacagagccatagcgtggaatcacttatttgggttgccgaagtagcggtcggagcgtgagttcttggtcaagccgccccttatccggttcctgtccgtgtctttgtccctcgttcacccttcgcggcacccttcatccccttgcttgcaggtaacatcctgggccacaagctggagtacaacttcaacagccacaacgtgtacatcaccgccgacaagcagaagaacggcatcaaggccaacttcaagatccgccacaacgtggaggacggcagcgtgcagctggccgaccactaccagcagaacacccccatcggcgacggccccgtgctgctgcccgacaaccactacctgagccaccagagcgccctgagcaaggaccccaacgagaagcgcgaccacatggtgctgctggagttcgtgaccgccgccatcgagggcaggtggagccacccgcagttcgagaagtaa

SEQ ID NO: 25 - protein, 227 aa, synthetic construct
VSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSHQSALSKDPNEKRDHMVLLEFVTAA